(19) United States
(12) Patent Application Publication
(10) Pub. No.: US 2018/0055816 A1
(43) Pub. Date: Mar. 1, 2018

(54) PYRANONAPHTHOQUINONE COMPOUNDS AND METHODS OF USE THEREOF

(71) Applicant: University of Kentucky Research Foundation, Lexington, KY (US)

(72) Inventors: Jon S. Thorson, Lexington, KY (US); Qing-Bai She, Lexington, KY (US); Bradley D. Anderson, Lexington, KY (US); Qing Ye, Lexington, KY (US); Markos Leggas, Lexington, KY (US); Yinan Zhang, Lexington, KY (US)

(21) Appl. No.: 15/669,864

(22) Filed: Aug. 4, 2017

Related U.S. Application Data
(60) Provisional application No. 62/371,123, filed on Aug. 4, 2016.

(51) Int. Cl. A61K 31/352; A61K 31/655; A61K 31/365; A61K 31/665

(52) U.S. Cl. CPC A61K 31/352; A61K 31/665; A61K 31/365; A61K 31/655

(57) ABSTRACT

Provided herein are pyranonaphthoquinone compounds and methods of using pyranonaphthoquinone compounds. The method of using the pyranonaphthoquinone compounds includes selectively inhibiting 4E-BP1 phosphorylation by administering at least one pyranonaphthoquinone or pyranonaphthoquinone analog to a subject in need thereof. The pyranonaphthoquinone compounds includes a structure according to Formula I:

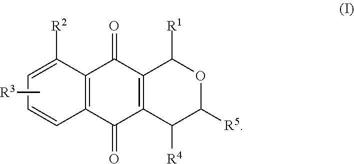

### RELATED APPLICATIONS

This application claims the benefit of U.S. Provisional Application Ser. No. 62/371,123, filed Aug. 4, 2016, the entire disclosure of which is incorporated herein by this reference.

### GOVERNMENT INTEREST

This invention was made with government support under grant numbers CA203257, CA175105, T32 DA016176, and CCTS UL1TRO00117 awarded by the National Institutes of Health (NIH), and grant number P30 CA177558 awarded by the National Cancer Institute (NCI). The government has certain rights in the invention.

### TECHNICAL FIELD

The presently-disclosed subject matter generally relates to pyranonaphthoquinone compounds, methods of forming pyranonaphthoquinone compounds, and methods of use thereof. In particular, certain embodiments of the presently-disclosed subject matter relate to pyranonaphthoquinone compounds, methods of forming pyranonaphthoquinone compounds, and methods for modulating cap-dependent translation and 4E-BP1 phosphorylation using pyranonaphthoquinone compounds.

### BACKGROUND

Disease progression and drug resistance to anticancer therapies is often associated with mutational activation of multiple signaling pathways that promote aberrant cell growth and metastasis. For example, the aggressiveness of diseases such as metastatic colorectal cancer (CRC) is in part driven by the aberrant expression of oncoproteins. As such, these diseases are difficult to treat and patients have few long term effective therapeutic options. At the molecular level, cap-dependent translation of the precursor oncogenic mRNAs is frequently activated. Specifically, this occurs via 4E-BP1 phosphorylation which, when not phosphorylated, functions as a mRNA translation repressor downstream from mTOR.

Novel therapeutic approaches include pharmacologic inhibition of proteins within signaling pathways as well as converging nodes like mTOR, which is activated in many cancers. The inhibition of mTOR has been of interest because it integrates multiple signals. However, the clinical efficacy of mTOR inhibitor drugs (e.g., rapamycin analogs) is limited. It is widely believed that this is largely attributed to their weak capacity to prevent phosphorylation of 4E-BP1 (a key translational repressor) which, when phosphorylated by mTOR, relieves its inhibitory control on elF4E-initiated cap-dependent translation of oncogenic mRNAs that drive oncoprotein production. Existing mTOR kinase (ATP-competitive) inhibitors non-selectively inhibit 4E-BP1 phosphorylation but also modulate the function of other mTOR-associated targets that may contribute to unwanted toxicities.

Accordingly, there exists a need for effective, selective inhibition of 4E-BP1 in cancer treatment.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a method of selectively inhibiting 4E-BP1 phosphorylation comprising administering at least one pyranonaphthoquinone or pyranonaphthoquinone analog to a subject in need thereof. In one embodiment the at least one pyranonaphthoquinone or pyranonaphthoquinone analog includes a structure according to Formula I:

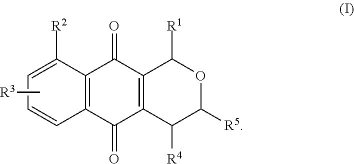

In another embodiment the at least one pyranonaphthoquinone or pyranonaphthoquinone analog is a frenolicin or frenolicin analog. In a further embodiment, the frenolicin analog includes a structure according to Formula II:

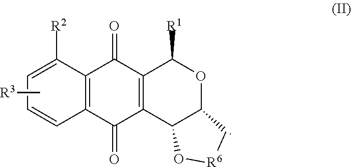

In still a further embodiment, the frenolicin analog includes a structure according to Formula III:

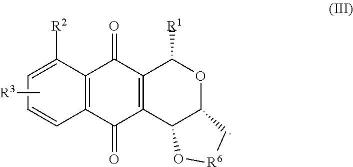

In on embodiment, the at least one pyranonaphthoquinone or pyranonaphthoquinone analog is a griseusin or griseusin analog. In another embodiment, the griseusin or griseusin analog includes a structure according to formula IV:

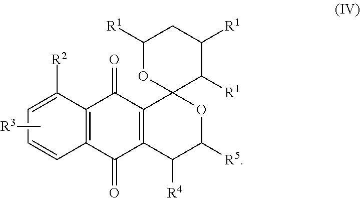

In some embodiments, administering the at least one pyranonaphthoquinone or pyranonaphthoquinone analog to the subject selectively inhibits 4E-BP1 phosphorylation. In some embodiments, administering the at least one pyranonaphthoquinone or pyranonaphthoquinone analog to the subject modulates 4E-BP1-regulated cap-dependent translation.

Also provided herein, in some embodiments, is a 4E-BP1 phosphorylation inhibitor comprising a pyranonaphthoquinone analog. In one embodiments, the pyranonaphthoquinone analog comprises a griseusin analog. In another embodiment, the griseusin analog includes a structure according to formula IV:

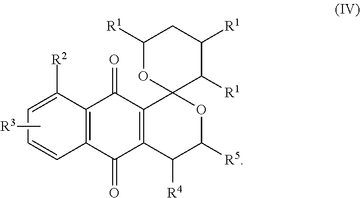

In one embodiment, the pyranonaphthoquinone analog comprises a frenolicin analog. In another embodiment, the frenolicin analog includes a structure according to Formula II:

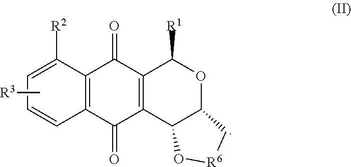

In a further embodiment, the frenolicin analog includes a structure according to Formula III:

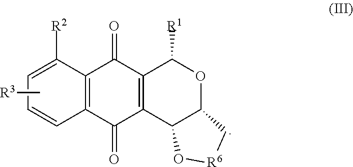

In some embodiments, the frenolicin analog is selected from the group consisting of an epi-frenolicin C1 analog, an epi-frenolicin ring A analog, an epi-frenolicin open D analog, and combinations thereof.

Further provided herein, in some embodiments, is a method of treating cancer comprising administering at least one pyranonaphthoquinone or pyranonaphthoquinone analog to a subject in need thereof. In one embodiment, the at least one pyranonaphthoquinone or pyranonaphthoquinone analog includes a structure according to Formula I:

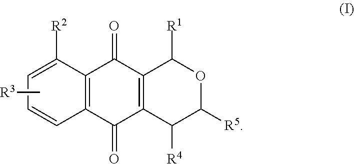

In another embodiment, the pyranonaphthoquinone analog comprises a griseusin analog. In a further embodiment, the pyranonaphthoquinone analog comprises a frenolicin analog.

Further features and advantages of the presently-disclosed subject matter will become evident to those of ordinary skill in the art after a study of the description, figures, and non-limiting examples in this document.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-C show structures of (A) UCF76-A, (B) frenolicin B, and (C) epi-frenolicin B.

FIG. 2 is a schematic view of translational control of colorectal cancer (CRC).

FIG. 3 shows a schematic of enantioselective syntheses of frenolicin analogs for SAR and probes.

FIG. 4 shows a schematic illustrating pyranonaphthaquinone inhibition of 4E-BP1 phosphorylation and tumor progression in vivo through a novel mechanism.

FIGS. 5A-C show graphs and images summarizing various frenolicin B (FB) representative structure-activity relationship (SAR) studies. (A) General divergent synthetic strategy to access FB and epi-FB analogs where the generic structure highlighted within the box illustrates points of diversification. This strategy has been employed to generate >40 analogs to date for subsequent in vitro hit identification and prioritization. (B) Inhibition of 4E-BP1 phosphorylation by representative analogs (left panel) and correlation of ROS production versus colorectal cancer cell line (HCT116) IC50 (right panel). GC refers to griseusin C (see FIG. 8A). (C) Structures of representative analogs with the panel (C) cytotoxicity-ROS correlation quadrant (B, right) noted in parentheses.

FIGS. 6A-C show graphs and images illustrating that a novel epi-FB analog yz-b47 is more potent and selective than FB in inhibiting 4E-BP1-regulated CRC cell growth. (A) Inhibition of 4E-BP1 phosphorylation upon exposure to different concentrations of FB and yz-b47 in HCT116 cells. (B) The half-maximal growth inhibitory concentration (IC₅₀) of FB and yz-b47 in HCT116 cells and mouse embryo fibroblasts (MEFs). (C) 4E-BP1/2 wild-type (WT) and double knockout (DKO) MEFs were treated with different concentrations of yz-b47 for 48 h. Results are illustrated as a percentage of cell number relative to DMSO-treated control cells. *p<0.02

FIGS. 7A-B show images illustrating the structure of various frenolicin and griseusin analogs. (A) C1-modified epi-FB analogs. (B) Ring A and open ring D analogs.

FIGS. 8A-C show structures and images illustrating naturally-occurring griseusins, a new griseusin synthetic strategy, and select structure-activity relationship (SAR). (A) Representative naturally-occurring griseusins. (B) New synthetic strategy for proposed griseusin SAR studies. (C) Griseusins produced to date via the strategy illustrated in panel (B) where those highlighted in green represent naturally-occurring griseusins (left panel). HCT116 cells were treated with differing concentrations of two representative members (right panel) as indicated for 12 h followed by immunoblot with the indicated antibodies (HCT116 IC₅₀s: GC, 127 nM; d43, 345 nM). Note that the correlation of ROS production versus colorectal cancer cell line (HCT116) IC₅₀ for GC is highlighted in FIG. 5C.

FIGS. 9A-C show graphs and images illustrating that a non-phosphorylated 4E-BP1 mutant (4E-BP1 4A) suppresses tumor growth and liver metastasis of CRC. (A) HCT116 cells expressing vector, 4E-BP1 WT or 4E-BP1-4A were transplanted into the right flank of athymic nude mice. Tumor volume was measured twice each week with the results presented as mean tumor volume±s.e.m. (n=5 mice/group). (B) Bioluminescence and GFP images of liver metastasis in athymic nude mice that were injected intrasplenically with HCT116-Luc/GFP cells expressing vector, 4E-BP1 WT or 4E-BP1 4A at week 3 post-injection. (C) Quantitative analysis of bioluminescence in liver metastasis as shown in (B) (n=5 mice/group). *P<0.03 for 4E-BP1 4A versus 4E-BP1 WT or vector.

FIGS. 10A-C. Elucidation of Streptomyces sp. RM-4-15 metabolites that effectively inhibit cap-dependent translation and colon cancer cell growth. (A) Inhibition of cap-dependent translation by Streptomyces sp. RM-4-15 bacterial extract. HCT116 CRC cells were transfected with a bicistronic luciferase reporter (upper diagram) that detects cap-dependent translation of the Renilla luciferase gene and cap-independent poliovirus IRES-mediated translation of the firefly luciferase gene. The transfected cells were treated with different concentrations of bacterial extract for 12 h. Cap-dependent renilla luciferase activity was normalized with cap-independent firefly luciferase activity. The results are expressed as the inhibition of cap-dependent translation relative to the untreated controls and presented as means±s.e.m. (n=3). (B) Inhibition of cap-dependent translation by representative pure metabolites (RM1-RM7) of Streptomyces sp. RM-4-15. RM1, UCF76-A; RM2, frenolicin B. (C) CRC cell cytotoxicity of UCF76-A. Cells were treated with UCF76-A for 72 h after which remaining viable cells were counted and the results are presented as a percentage of cell number relative to DMSO-treated control cells.

FIG. 11 shows a graph illustrating that frenolicin B exhibits potent cytotoxic activity against CRC cells. CRC cells were treated with different concentrations of FB for 72 hours. The number of viable cells were counted and the results are presented as a percentage of cell number relative to DMSO-treated control cells.

FIG. 12 shows a comparative Western blot analysis of HCT116 cells treated with mechanistically defined agents (MK2206, AKT inhibitor; PD0325901, MEK inhibitor; rapamycin, mTORC1 inhibitor; AZD8055, mTORC1/2 kinase inhibitor) and Streptomyces RM-4-15 pyranonaphthoquinones UCF76-A and FB. HCT116 cells were treated with 1 μM MK2206 and 100 nM PD0325901 alone and in combination, 100 nM rapamycin, 0.5 μM AZD8055, 2 μM UCF76-A or 2 μM FB for 12 h followed by Western blot analysis for the indicated proteins.

FIG. 13 shows a graph illustrating that FB and UCF76-A are more potent than mTOR inhibitors in inhibiting CRC cell growth. HCT116 cells were treated with 100 nM rapamycin, 0.5 μM AZD8055, 0.5 μM FB, 0.5 μM UCG76-A or vehicle control for the indicated day followed by counting the number of viable cells. *P<0.001 for FB or UCF76-A versus rapamycin and AZD8055. Statistical significance was determined by the Student's t-test.

FIG. 14 shows a Western blot analysis indicated that FB and UCF76-A selectively inhibit 4E-BP1 phosphorylation in DLD-1 colon cancer cells and MDA-MB-231 breast cancer cells. Cells were treated with 100 nM rapamycin, 0.5 μM AZD8055, 2 μM FB, 2 μM UCF76-A or DMSO control for 12 hours followed by Western blot analysis for the indicated proteins.

FIG. 15 shows structures of representative synthetic pyranonaphthoquinone analogs, according to an embodiment of the disclosure.

FIGS. 16A-C. Selective inhibition of 4E-BP1 phosphorylation by naturally-occurring and synthetic pyranonapthoquinones and correlation to CRC cell cytotoxicity. (A) HCT116 cells were treated with different concentrations of the indicated compounds for 72 h and the results are presented as a percentage of viable cell number relative to DMSO-treated control cells. (B) and (C) HCT116 cells were treated with 2 μM of the indicated compounds (B) or with different concentrations of FB and 12 (C) for 12 h followed by Western blot analysis.

FIGS. 17A-B show a graph and image illustrating that dephosphorylation of 4E-BP1 by FB and related analogs induces 4E-BP1 binding to eIF4E and inhibits cap-dependent translation. (A) HCT116 cells were treated with 2 μM of the indicated compounds for 12 hours. Cell lysates were precipitated with m⁷GTP sepharose beads followed by Western blot analysis for the indicated proteins. (B) HCT116 cells were transfected with a bicistronic luciferase reported that detects cap-dependent translation of the Renilla luciferase gene and cap-independent poliovirus IRES-mediated translation of the firefly luciferase gene. After 24 hours, cells were treated with 2 μM of the indicated compounds or DMSO as control for 12 hours. Each luciferase activity was measured using a dual-luciferase assay kit, and the ratio of Renilla/Firefly luciferase activity was calculated and expressed as a percentage of the cap-dependent translation activity found in the DMSO-treated control cells. The graphic data are presented as mean±s.e.m. (n=3 technical replicates per condition). *P<0.01 versus DMSO; NS, not significant, Statistical significance was determined by the Student's t-test.

FIGS. 18A-D show graphs and images illustrating that compound 12 of the instant disclosure is more potent that FB in inhibition of cap-dependent translation and induction of apoptosis. (A) HCT116 cells were treated with different concentrations of FB and 12 for 12 hours. Cell lysates were precipitated with m⁷GTP sepharose beads followed by Western blot analysis for the indicated proteins. (B) Inhibition of cap-dependent translation activity in HCT116 cells that were treated with the indicated compounds or DMSO as control for 12 hours was determined and analyzed as in FIG. 10A. (C) and (D) HCT116 cells were treated with different concentrations of FB and 12 for 72 hours. Apoptotic cells were analyzed by propidium iodide/Annexin V staining and flow cytometry (C). The results are expressed as the increased levels of apoptosis (D) by subtracting each of the DMSO-treated controls. All graphic data are presented as mean±s.e.m. (n=3 technical replicates per condition). *P<0.001 for 12 versus FB. Statistical significance was determined by the Student's t-test.

FIG. 19 shows HCT116 cells with stable expression of control shRNA or 4E-BP1 shRNA were treated with 2 μM FB, 2 μM 12 or DMSO control for 24 h. Cap-dependent translation activity was determined as in FIG. 10A and normalized with DMSO-treated shRNA controls. The graphic data are presented as mean±s.e.m. (n=3). *P<0.01 versus DMSO; ns, not significant, Statistical significance was determine by the Student's t-test.

FIGS. 20A-D show structures, images, and sequences of probes illustrating that FB directly targets Prx1 and Grx3. (A) Structures of inactive d5 and active d7 probes. (B) HeLa cell lysates were incubated with FB-based biotinylated active d7 and inactive d5, followed by pull-down with streptavidin-agarose. The precipitates were resolved by SDS-PAGE, and the gel was stained with Coomassie blue. (C) Sequences illustrating amino acids and peptides of Prx1 and Grx3 identified by mass spectrometry analysis. The unique bands as shown by d7-pull down in the experiment for FIG. 20B were excised and subjected to in-gel tryptic digestion and analysis by MS, wherein the identified amino acids and peptides are shown in red characters. (D) Western blot illustrating that HCT116 cell lysates were incubated with different concentrations of d5 or d7 in the absence or presence of a ten-fold excess of FB for 3 h, followed by pull-down with streptavidin-agarose and Western blot analysis for the indicated proteins.

FIGS. 21A-D show images illustrating that the active probe d7 binds to and co-localizes with Prx1 and Grx3. (A) and (B) The recombinant Prx1 (A) or Grx3 (B) was incubated with d7 for 2 hours, followed by streptavidin pull down and Western blot analysis for the indicated proteins. (C) and (D) HCT116 cells were incubated with 25 μM d5, 25 μM d7 or DMSO as control for 5 hours, followed by confocal sections of the cells staining for Prx1 (C) or Grx3 (D) (red), biotin (green) and DAPI (blue). Scale bars, 10 μm.

FIGS. 22A-B show a graph and table illustrating that FB and compound 12 potently inhibit Prx1 catalytic activity. The recombinant Prx1 protein was incubated with the indicated concentration of FB, 12, or conoidin A for 1 hour. Data are presented as mean±s.e.m. (n=3 technical replicates per condition). *P<0.01 for Snail versus vector. Statistical significance was determined by the Student's t-test.

FIGS. 23A-B show graphs illustrating the effect of FB and its analogs on modulation of GSH and GSSG. HCT116 cells were treated with 2 μM of the indicated compounds or DMSO as control for 5 hours, followed by measurement of cellular GSH (A) or GSSG (B) levels. Results are expressed as a percentage of GSH levels relative to the value found in DMSO-treated control cells (A), or as a fold increase in GSSG levels over the value found in the DMSO-treated control cells (B). All graphic data are presented as mean±s.e.m. (n=3 technical replicates per condition). *P<0.01 versus DMSO; NS, not significant. Statistical significance was determined by the Student's t-test.

FIGS. 24A-B. FB directly targets Prx1 and Grx3.(A) and (B) HCT116 cells transfected with wild-type (WT) Prx1 (A) or Grx3 (B) and their mutants or vector control were incubated with d7 for 3 h, followed by pull-down with streptavidin-agarose and Western blot analysis for the precipitated proteins and whole cell lysates (WCL).

FIGS. 25A-C show images of Western blot analyses illustrating that FB targets Prx1 on Cys83 and Grx3 on both Cys 159 and Cys261. HCT 116 cells were transfected with Myc-tagged Prx1 or Grx3 wild-type (WT), their mutants, or control vector for 36 hours. Cell lysates were incubated with d7 for 2 hours, followed by immunoprecipitation with myc antibody (A and C) or streptavidin pull down (B) and Western blot analysis for the indicated proteins. WCL, whole cell lysates.

FIGS. 26A-B. FB induces ROS production to repress 4E-BP1 phosphorylation and tumor growth in vivo. (A) The extracellular H₂O₂ level was determined in HCT116 cells treated with 2 μM of the indicated compounds or DMSO control for 5 h. (B) HCT116 cells were treated with 2 μM of the indicated compounds or DMSO control for 1 h, followed by incubation with CellROX Deep Red reagent and flow cytometry analysis of ROS. Results are expressed as a fold increase in ROS levels over the value found in the DMSO-treated control cells. The graphic data (A, B) are presented as mean±s.e.m. (n=3). *P<0.01 versus DMSO; NS, not significant. Statistical significance was determine by the Student's t-test.

FIG. 27 shows an image illustrating an image of a Western blot analysis for the indicated proteins in HCT116 cells that were treated with different concentrations of H₂O₂ for 2 hours.

FIG. 28 shows an image illustrating a Western blot analysis of HCT116 cells pre-treated with 400 μM N-acetyl-L-cysteine for 1 h before treatment with 2 μM of the indicated compounds or DMSO control for 6 h.

FIGS. 29A-C show graphs and images illustrating that silencing Prx1 and Grx3 induces H₂O₂ and inhibits 4E-BP1 phosphorylation. (A) and (B) HCT116 cells with stable expression of two different sets of Prx1 (A), Grx3 (B) shRNAs or control shRNA (shCtrl) were lysed and analyzed by Western blot for the indicated proteins. (C) The extracellular H₂O₂ level was determined in HCT116 cells with stable expression of Prx1 shRNA and Grx3 shRNA alone and in combination or control shRNA. The graphic data are presented as mean±s.e.m. (n=3 technical replicates per condition). *P<0.01 for combination of Prx1 and Grx3 shRNAs versus Prx1, Grx3, or control shRNA. Statistical significance was determined by the Student's t-test.

FIGS. 30A-B. FB induces ROS production to repress 4E-BP1 phosphorylation and tumor growth in vivo. (A) Western blot analysis of human fibroblasts obtained from a Zellweger (GM13267) or a corresponding control patient (with Ehlers-Danlos syndrome (GM15871)) treated with 2 μM of FB or 12 for 6 h. (B) Western blot analysis of HCT116 cells with stable expression of control shRNA or TSC2 shRNA treated with 2 μM of FB or 12 for 6 h.

FIG. 31 shows a schematic illustrating the formation of compound 14.

FIG. 32 shows a graph illustrating tumor size of mice bearing HCT116 or DLD-1 CRC xenograft tumors that were treated with 14 (14 mg/kg, five times/week) or vehicle control. Tumor size was measured by caliper two times per week. The results are presented as the mean tumor volume±s.e.m. (n=8 mice/group). ^{#}P<0.02; ^{##}P<0.001 for 14 versus vehicle control by the Student's t-test.

FIG. 33 shows a graph illustrating that chronic treatment with compound 14 does not cause significant weight loss in mice. Mice bearing HCT116 or DLD-1 CRC xenograft tumors were treated with compound 14 at 14 mg/kg or with vehicle control once per day for 5 consecutive days each week. The mouse body weight was measured twice per week in control and treated groups using a weighing scale. The results represent the mean body weight±s.e.m. (n=8 mice/group). *P>0.05 for 14 versus vehicle control in HCT116 or DLD-1 xenograft tumors. Statistical significance was determined by the Student's t-test.

FIGS. 34A-B. FB induces ROS production to repress 4E-BP1 phosphorylation and tumor growth in vivo. (A) Western blot analysis of representative tumors collected from mice in FIG. 32 6 h after the final treatment with 14 or vehicle control. (B) A proposed model for the anticancer mechanism of FB through targeting Prx1/Grx3 to induce ROS accumulation leading to inhibition of mTORC1/4E-BP1-mediated tarnaltion.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter. Unless defined otherwise, all technical and scientific terms used herein have the same meaning as is commonly understood by one of skill in the art to which the invention(s) belong.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9): 1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter includes methods and compounds for inhibiting 4E-BP1 phosphorylation. In some embodiments, the compound includes a 4E-BP1 inhibitor. For example, in one embodiment, the inhibitor is a pyranonaphthoquinone or pyranonaphthoquinone analog. In another embodiment, the inhibitor is of the structure represented by Formula I:

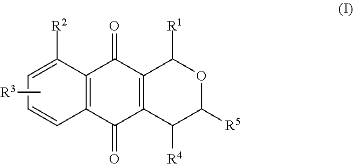

where R¹ includes, but is not limited to, H; C₁-C₆ alkyl (e.g., CH₂CH₃, CH₂CH₂CH₃, CH(CH₃)₂); (CH₂)ₙX, where n is between 0 and 6 and X is N₃, CN, CH₃, aryl (e.g., C₆H₅), triazole (e.g., C₂H₂N₃), alkyl-substituted triazole, piperidyl (e.g., (CH₂)₅N), morpholinyl (e.g., O(CH₂CH₂)₂N), tetrahydropyranyl (e.g., C₅H₉O), cyclohexyl (e.g., C₆H₁₁), halogen-substituted aryl (e.g., C₆H₄F, C₆H₃F₂, C₆H₄Cl, C₆H₄Br,); alkyl-substituted aryl (e.g., C₆H₄(CH₃)), alkoxyl-substituted aryl (e.g., C₆H₄(OCH₃)), hydroxyl-substituted aryl (e.g., C₆H₄(OH)), amino-substituted aryl (e.g., C₆H₄(NH₂)), pyridinyl (e.g., C₅H₄N), diazinyl (e.g., C₄H₃N₂), triazinyl (e.g., C₃H₂N₃), C(O)C₆H₅, or OCH₂C₆H₅; alkenyl (e.g., CH₂CHCH₂); alkynyl (e.g., CH₂CH₂CCH); where R² includes, but is not limited to, H, OH, alkoxy (e.g., OCH₃), halogen, or amine; where R³ includes, but is not limited to, H, OH, or halogen; where R⁴ includes, but is not limited to, H or OH; and where R⁵ includes, but is not limited to, CH₂Y, CH₂CH₂Z, or CH₂C(O)Z, where Y is CN, CH₂OR, CH₂NHR, COOR, or CONHR, and where Z is a bond between R⁴ and R⁵, forming a five membered ring with the carbon atoms to which R⁴ and R⁵ are attached.

In certain embodiments, suitable structures corresponding to the chemical formulas disclosed for R¹ above include, but are not limited to:

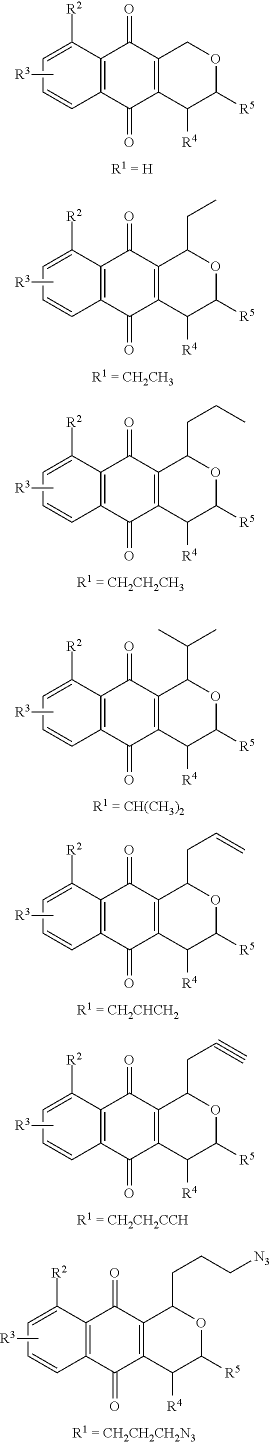

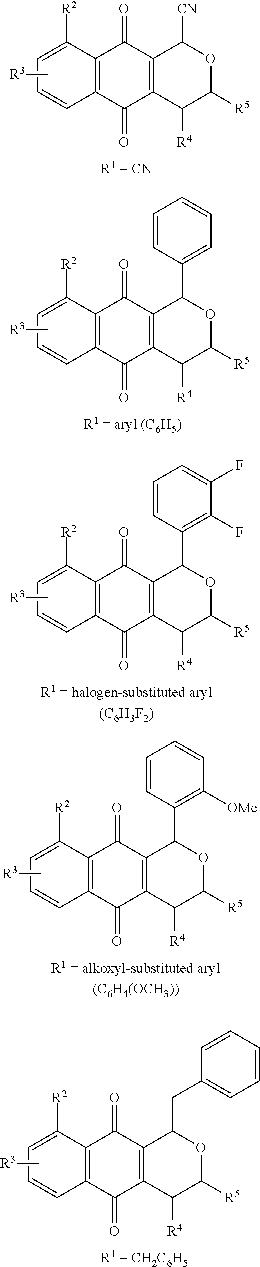

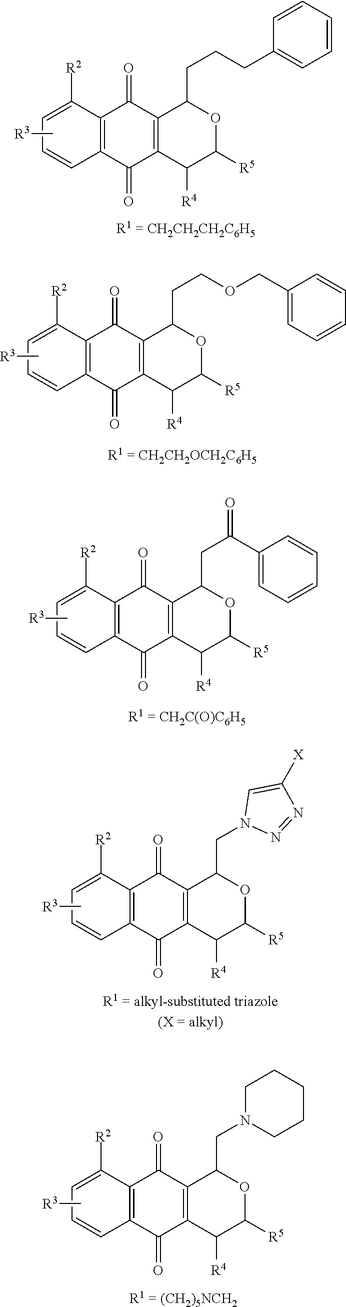

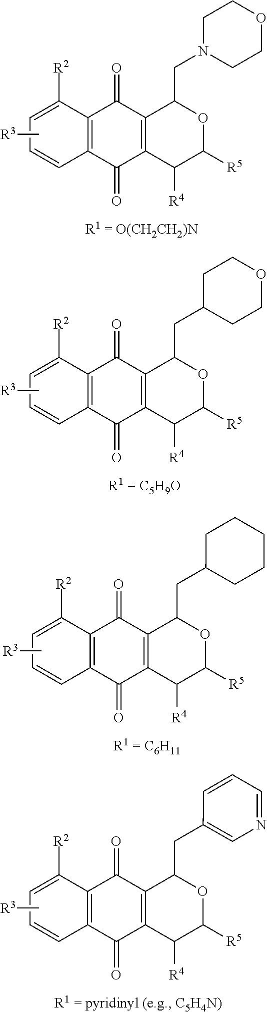

In some embodiments, suitable structures corresponding to the chemical formulas disclosed for R² and R³ above include, but are not limited to:

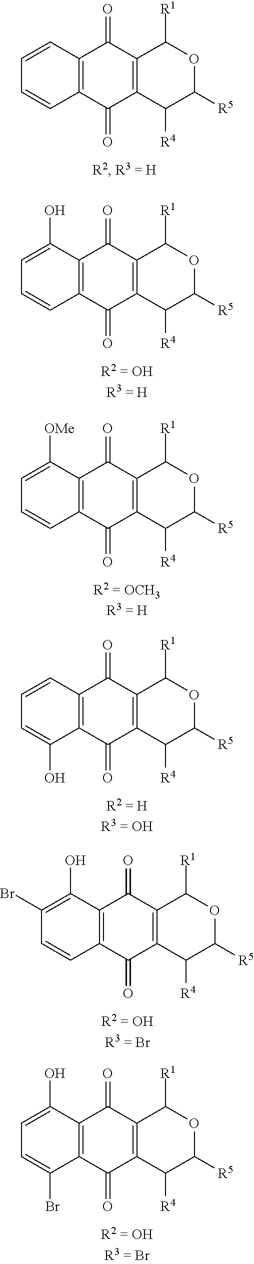

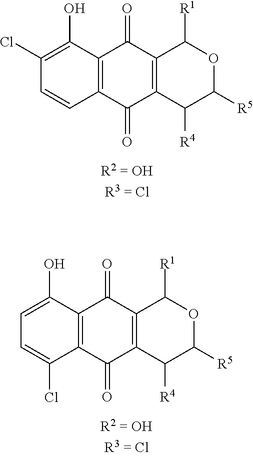

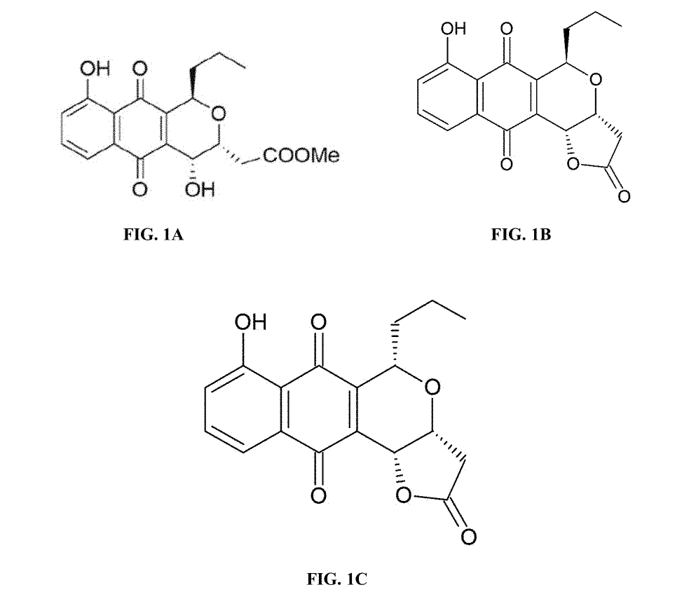

Turning to R⁴ and R⁵, in some embodiments, as discussed above, the groups are separate and do not form a ring. One example of these embodiments includes UCF76-A (FIG. 1A), where R¹ is CH₂CH₂CH₃, R² is OH, R³ is H, R⁴ is OH and R⁵ is CH₂C(O)OCH₃. In another example R¹ is CH₂CH₂CH₃, R² is OH, R³ is H, R⁴ is OH and R⁵ is CH₂C(O)OH. Alternatively, in some embodiments, where R⁴ and R⁵ of Formula I form the five membered ring discussed above, the inhibitor includes a structure according to one of the stereoisomers represented by Formula II or Formula III below:

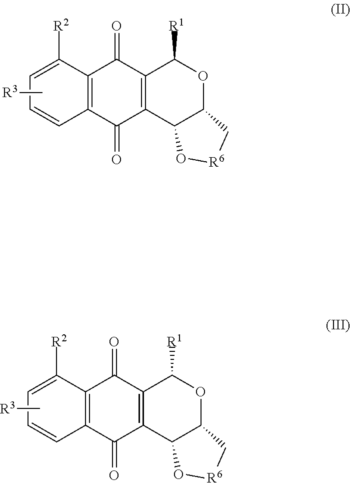

where R¹, R², and R³, are as defined above with regard to Formula I, and R⁶ includes, but is not limited to, CH₂ or CO. In one embodiment, the inhibitor according to Formula II includes frenolicin B (FIG. 1B) or an analog thereof. In another embodiment, the inhibitor according to Formula III includes epi-frenolicin B (FIG. 1C) or an analog thereof.

As used herein, the term analog refers to any suitable combination of variable groups according to the Formulas disclosed herein. Additionally, in some embodiments, the term analog refers to stereoisomers of the inhibitor. For example, the down stereo bonds of R⁴ and/or R⁵ in the open configuration, or the down stereo bonds of the five member ring including R⁶, may be replaced with up stereo bonds in any one or more of the embodiments disclosed herein. Suitable structures according to Formula II, where R¹ is attached through an up stereo bond, and Formula III, where R¹ is attached through a down stereo bond, include, but are not limited to:

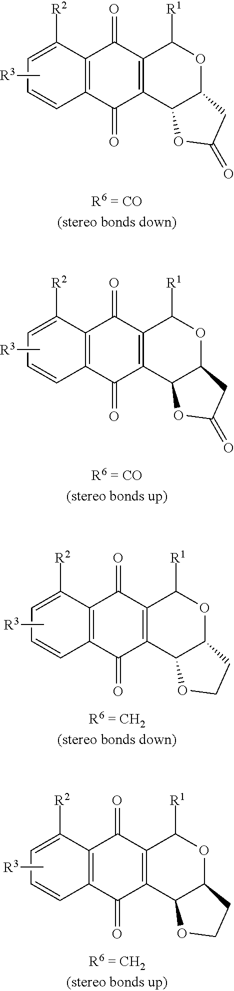

In some embodiments, the inhibitor of the instant disclosure includes a griseusin and/or griseusin analog. For example, in one embodiment, the griseusin and/or griseusin analog includes the structure according to Formula IV below:

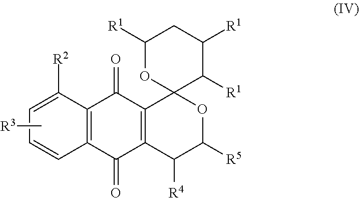

Where each R¹ independently includes, but is not limited to, H, O, OH, OCH₃, OC(O)CH₃, N₃, NH₂, halide, or C₁-C₆ alkyl (e.g., CH₃); where R² includes, but is not limited to, H, OH, alkoxy (e.g., OCH₃), halogen, or amine; where R³ includes, but is not limited to, H, OH, or halogen; where R⁴ includes, but is not limited to, H or OH; and where R⁵ includes, but is not limited to, CH₂Y, CH₂CH₂Z, or CH₂C(O)Z, where Y is CN, CH₂OR, CH₂NHR, COOR, or CONHR, and where Z is a bond between R⁴ and R⁵, forming a five membered ring with the carbon atoms to which R⁴ and R⁵ are attached.

In one embodiment, the griseusen of Formula IV is griseusen C, which has the following structure:

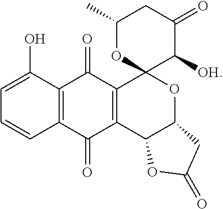 [SMILES: C[C@@H]1CC(=O)[C@@H](O)[C@]2(O1)O[C@@H]1CC(=O)O[C@@H]1C1=C2C(=O)c2c(O)cccc2C1=O]

In another embodiment, the griseusen of Formula IV is a griseusen C analog, which is a stereoisomer of griseusen C and/or has a structure according to Formula V below:

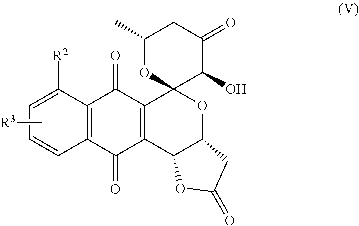

and stereoisomers thereof, where R² and R³ are as discussed above with regard to Formula IV. In a further embodiment, griseusen C analogs include, but are not limited to:

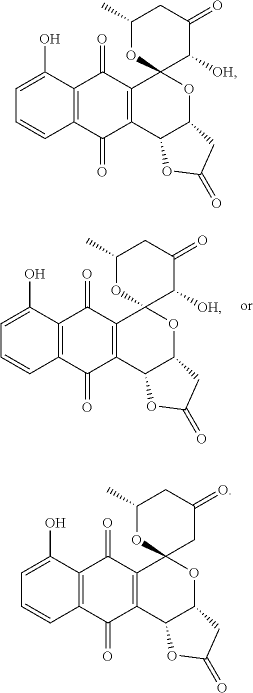

Additionally or alternatively, in one embodiment, the griseusen of Formula IV is griseusen A, which has the following structure:

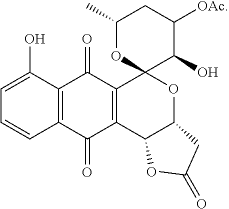

In another embodiment, the griseusen of Formula IV is a griseusen A analog, which is a stereoisomer of griseusen A and/or has a structure according to Formula VI below:

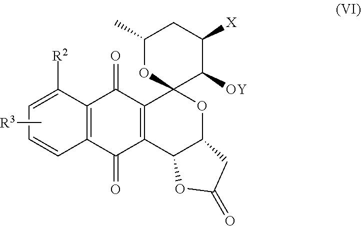

and stereoisomers thereof, where R² and R³ are as discussed above with regard to Formula IV; X is OAc, OH, or O; and Y is H or SG. For example, one griseusen A analog includes 4′-deacetyl-GA, which has the following structure:

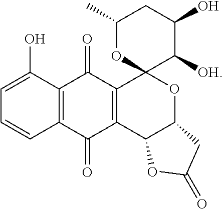 [SMILES: C[C@@H]1C[C@@H](O)[C@@H](O)[C@]2(O1)O[C@@H]1CC(=O)O[C@@H]1C1=C2C(=O)c2c(O)cccc2C1=O]

As will be appreciated by those skilled in the art, the structures discussed above are for illustration only and are not intended to limit the scope of the instant disclosure. Accordingly, other pyranonaphthoquinone and pyranonaphthoquinone analogs are expressly contemplated herein, including, but not limited to, frenolicin analogs, griseusin B, griseusin D, griseusin E, griseusin F, griseusin G, and analogs thereof.

In some embodiments, one or more of the pyranonaphthoquinones or pyranonaphthoquinone analogs described herein is arranged and disposed to bind and/or inhibit any suitable target. Suitable targets include, but are not limited to, targets which are overproduced in one or more cancers and/or which play a key role in one or more parasitic-type diseases. For example, in one embodiment, one or more of the pyranonaphthoquinones or pyranonaphthoquinone analogs described herein binds glutaredoxin, peroxiredoxin, or a combination thereof. As used herein, the terms “glutaredoxin” and “peroxiredoxin” include any suitable isoform thereof, such as, but not limited to, glutaredoxin 3, peroxiredoxin 1, peroxiredoxin 2, any other isoform thereof, or a combination thereof. In another embodiment, the binding of the one or more of the pyranonaphthoquinones or pyranonaphthoquinone analogs described herein inhibits glutaredoxin and/or peroxiredoxin. Without wishing to be bound by theory, it is believed that the compounds disclosed herein represent the first reported inhibitor of glutaredoxing to date. Again, without wishing to be bound by theory, it is believed that in certain embodiments the pyranonaphthoquinones or pyranonaphthoquinone analogs described herein also provide the most potent inhibitor of peroxiredoxin to date.

Additionally or alternatively, in some embodiments, as discussed above, the pyranonaphthoquinones or pyranonaphthoquinone analogs described herein form 4E-BP1 phosphorylation inhibitors. More specifically, in contrast to the previously held belief that pyranonaphthoquinones or pyranonaphthoquinone analogs were Akt inhibitors, the instant inventors surprisingly discovered that one or more of the pyranonaphthoquinones or pyranonaphthoquinone analogs disclosed herein inhibit 4E-BP1 phosphorylation through inhibition of Prx1 and/or Grx3, without or substantially without inhibiting Akt. That is, pyranonaphthoquinones or pyranonaphthoquinone analogs disclosed herein inhibit 4E-BP1 phosphorylation in a manner that is mechanistically distinct to existing mTOR inhibitors.

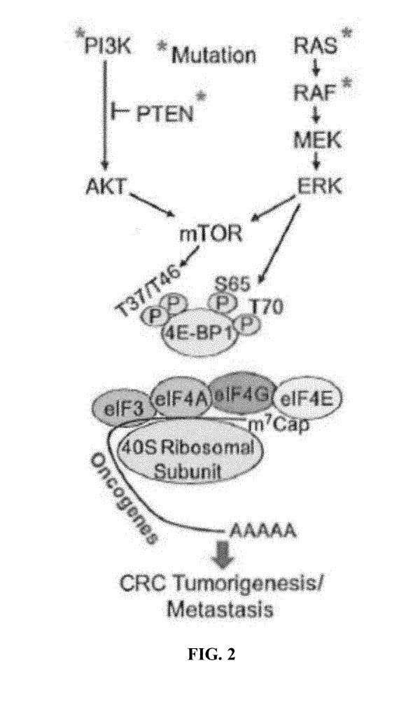

Without wishing to be bound by theory, it is believed that deregulation of cap-dependent translation downstream of mTOR at the level of 4E-BP1/elF4E is a key to tumor formation and metastatic progression. More specifically, translation of key oncogenic mRNAs is strongly dependent on the mRNA cap-binding protein elF4E (FIG. 2). Consequently, expression of these oncogenic mRNAs is preferentially and disproportionately affected by elF4E availability and is sensitive to the alteration of its levels. Indeed, as in many cancers, CRC progression and poor prognosis is associated with substantially elevated elF4E levels due to elF4E upregulation and reduction in the levels of the active non-phosphorylated repressor 4E-BP1.

In this regard, the instant inventors recently discovered that activated signaling via the PI3K/AKT and RAS/RAF/MEK/ERK pathways cooperate to promote CRC progression by convergent phosphorylation (inactivation) of 4E-BP1. Additionally, the instant inventors have demonstrated that 4E-BP1 phosphorylation-mediated oncogene translation functions as a critical node that integrates oncogenic signals of the AKT and ERK pathways for CRC tumorigenesis and metastasis. Moreover, the instant inventors have found that CRC resistance to upstream kinase targeted therapy is associated with incomplete inhibition of 4E-BP1 phosphorylation. That is, due to cooperation between the pathways, inhibition of Akt or Erk alone is insufficient to provide complete inhibition of 4E-BP1 phosphorylation, and therefore, is insufficient to suppress tumor growth and metastasis.

However, the pyranonaphthoquinones or pyranonaphthoquinone analogs disclosed herein form what is believed to be the first selective inhibitor of 4E-BP1 phosphorylation. Since the novel mechanism for inhibiting 4E-BP1 phosphorylation disclosed herein does not rely on Akt or Erk inhibition, the pyranonaphthoquinones or pyranonaphthoquinone analogs of the instant disclosure eliminate the redundant phosphorylation issues associated therewith. Additionally, pharmacoloqic activation of 4E-BP1, via pharmacological inhibition of 4E-BP1 phosphorylation, disrupts cap-dependent translation by blocking converging oncogenic signals at a key node. Therefore, in one embodiment, administering at least one pyranonaphthoquinone or pyranonaphthoquinone analog modulates 4E-BP1-regulated cap-dependent translation and provides a treatment strategy for advanced CRC and/or other major cancers. Thus, in certain embodiments, the pyranonaphthoquinones or pyranonaphthoquinone analogs disclosed herein form novel anticancer drugs, where direct targeting of 4E-BP1 phosphorylation-mediated oncogene translation represents a novel strategy for cancer drug development and therapy.

Accordingly, also provided herein, in some embodiments, is a method of treating cancer including administering at least one of the pyranonaphthoquinones and/or pyranonaphthoquinone analogs to a subject in need thereof. Suitable pyranonaphthoquinones or pyranonaphthoquinone analogs include any of the compounds disclosed herein, such as, but are not limited to, frenolicin B (FB), epi-FB, griseusin, analogs thereof, or a combination thereof. In one embodiment, the method provides selective inhibition of 4E-BP1 phosphorylation.

As used herein, the term “cancer” refers to all types of cancer or neoplasm or malignant tumors found in animals, including leukemias, carcinomas, melanoma, and sarcomas. For example, the cancer may include, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, colorectal cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer. In some particular embodiments, the cancer is colorectal cancer (CRC).

Further provided herein, in some embodiments, is a method of treating a parasitic-type disease. In some embodiments, the method of treating a parasitic-type disease includes administering at least one pyranonaphthoquinone or pyranonaphthoquinone analog to a subject in need thereof. Suitable pyranonaphthoquinones or pyranonaphthoquinone analogs include, but are not limited to, frenolicin B (FB), epi-FB, griseusin, an analog of FB, epi-FB, or griseusin, or a combination thereof.

As used herein, the term “parasitic-type disease” refers to any disease caused by or resulting from a parasite. For example, in one embodiment, parasitic-type disease includes malaria. In another embodiment, one or more of the pyranonaphthoquinones or pyranonaphthoquinone analogs described herein provide anti-parasitic-type disease function by inhibiting one or more of the targets disclosed herein. More specifically, in certain embodiments, one or more of the pyranonaphthoquinones or pyranonaphthoquinone analogs described herein inhibit peroxiredoxin, which provides potent anti-malarial function. This peroxiredoxin inhibition may, in some embodiments, provide increased antimalarial potency as compared to existing malaria drugs/treatments.

The presently-disclosed subject matter also includes, in some embodiments, method of forming a pyranonaphthoquinone or pyranonaphthoquinone analog. In one embodiment, the method includes an enantioselective syntheses of frenolicin analogs, as described in greater detail in the Examples below. In another embodiment, the method includes a divergent process for the synthesis of both FB and epi-FB. In a further embodiment, the method provides Lewis acid catalyst optimization to facilitate increased control over diastereoselectivity in the key oxa-Pictet-Spengler reaction, thereby providing access to all core stereoisomers. Additionally or alternatively, the method provides optimization of the culminating demethylation (a key deprotection step) to avoid epimerization that notably plagued prior syntheses. Methods for synthesizing griseusin and giseusin analogs are also provided herein.

Still further provided herein, in some embodiments, is a molecular probe that facilitates understanding of 4E-BP1 in tumorigenesis and metastasis. In one embodiment, the molecular probe binds to SEQ ID NO: 1 and/or SEQ ID NO: 2.

### EXAMPLES

### Example 1—Frenolicin Synthesis

Enantioselective Syntheses of Frenolicin Analogs for SAR and Probes.

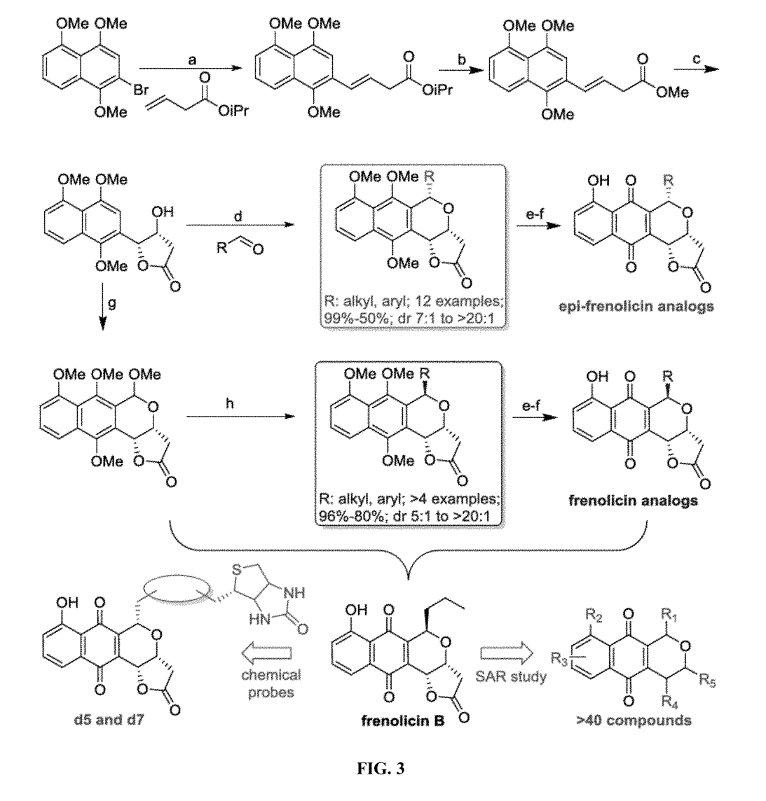

Referring to FIG. 3, enantioselective syntheses of frenolicin analogs for SAR and probes includes the following reagents and conditions: (a) 2 mol % Pd(tBU₃P), Cy₂NM3, Toluene, 100° C., 16 h, 84%; (b) TsOH, MeOH, room temp, 16 h, 75%; (c) K₃Fe(CN)₆, K₂OsO₄, K₂CO₃, NaHCO₃, (DHQD)₂PHAL, CH₃SO₂NH₂, t-BuOH/H₂O, 0° C.-room temp, 40 h, 63%; (d) 50 mol % Cu(OTf)₂, DCM, 16 h, 0° C.-room temp, 16 h, 99-50%; (e) CAN, CH₃CN/H₂O, 0° C., 10 min; (f) BCl₃, CH₂Cl₂, −78° C., 1-2 h, 60-40% for the two steps; (g) trimethyl orthoformate, EtAlCl₂, CH₂Cl₂, −20° C., 16 h, 65%; (h) Nucleophiles, Lewis Acid, CH₂Cl₂, −78° C.-room temp, 1-16 h; 96-80%.

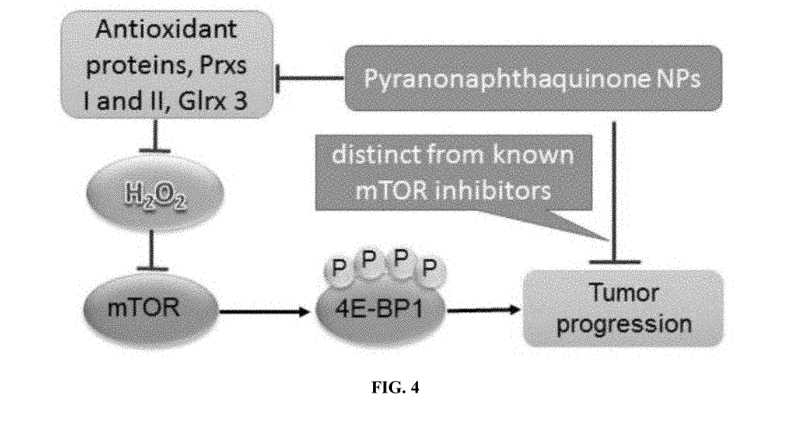

A schematic example of the mechanism of pyranonaphthaquinone inhibition of 4E-CP1 phosphorylation and tumor progression in vivo is shown in FIG. 4.

Synthesis and Preliminary Evaluation of Novel FB-Based Analogs.

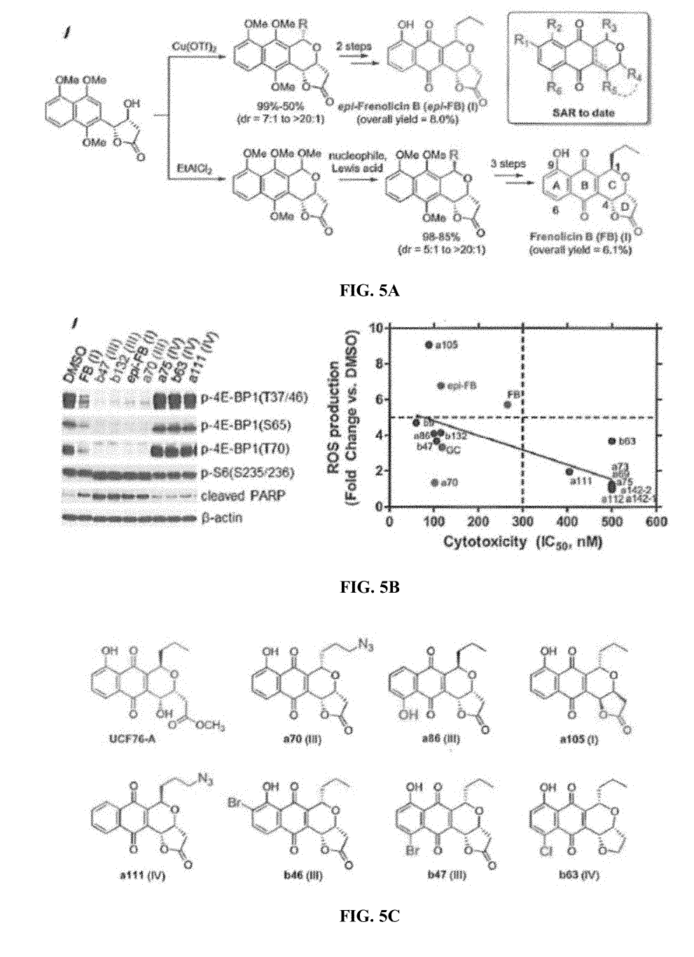

Prompted by the potentially novel mechanism of action outlined above, the instant inventors developed an efficient divergent strategy for the synthesis of both FB and epi-FB to allow for further exploration of the pyranonaphthoquinone SAR (FIG. 5A) (17). This strategy encompasses two notable advances when compared to prior reported syntheses (42-46): 1) Lewis acid catalyst optimization to enable exquisite control over diastereoselectivity in the key oxa-Pictet-Spengler reaction (and thereby provide access to all core stereoisomers) and 2) optimization of the culminating demethylation (a key deprotection step) to avoid epimerization (an issue that notably plagued prior syntheses). This much improved divergent synthetic strategy has enabled the synthesis of >40 novel analogs for subsequent in vitro hit identification and prioritization assays.

Based on the preliminary mechanistic studies with naturally-occurring FB metabolites described above, an initial streamlined compound prioritization strategy that requires 90% inhibition of 4E-BP1 phosphorylation at 1 1.LM of test compound was selected as the initial filter. Compounds that pass this single dose filter are then tested in a CRC (HCT116) in vitro cytotoxicity assay and, in parallel, an in vitro assay to assess propensity for intracellular reactive oxygen species (ROS) production using the redox sensitive probe CeIIROX® Deep Red reagent (Invitrogen) by flow cytometry analysis (47). The rationale for inclusion of ROS induction as a key criterion stems from the well-established liabilities of quinones (namely, as oxidants and electrophiles) (48-50) and the demonstrated ability of ROS to activate key signaling pathways of relevance to cancer (51, 52). These data are then correlated as illustrated in FIG. 5B where it is believed that compounds in quadrant III (high anticancer potency, low ROS generation) present highly specific and potent 4E-BP1 phosphorylation inhibitors with limited quinone-based non-specific liabilities.

Using this general strategy, initial SAR analyses (FIG. 5) revealed: 1) importance of the juglone (the fused A and B ring hydroxynaphthoquinone) core with tolerance to regiospecificity of the corresponding hydroxyl (C6 or C9); 2) tolerance to modest C6 and C8 substitution of the A ring; 3) notable tolerance to C1 modification of the C ring; 4) a slight improvement in activity with ‘open’ D-ring esters (e.g., UCF76-A) including those lacking the C4 hydroxyl; and 5) a trend toward increased 4E-BP1-dependent cytotoxicity and reduced toxicity indicators with epi-FB analogs. Based upon this cumulative analysis, higher priority analogs include: a70, b47, a86 and b132 (FIGS. 5B-C), where a positive correlation between 4E-BP1 phosphorylation inhibitory potency and HCT116 cell line cytotoxicity potency as indicated by growth inhibition and induction of cleaved PARP was also apparent (FIG. 5B). It is also important to note that, while azide anion (such as NaN₃) is known as scavenger of singlet oxygen (53), organic azides do not display similar properties. Thus, a70 clearly stands out as uniquely active among this priority set and, in conjunction with its inactive structural comparator all1, notably presents an outstanding reagent set for the chemoselective pulldown target identification studies presented herein.

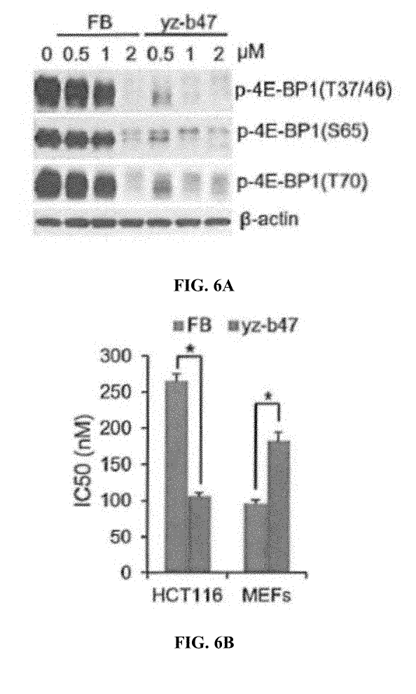

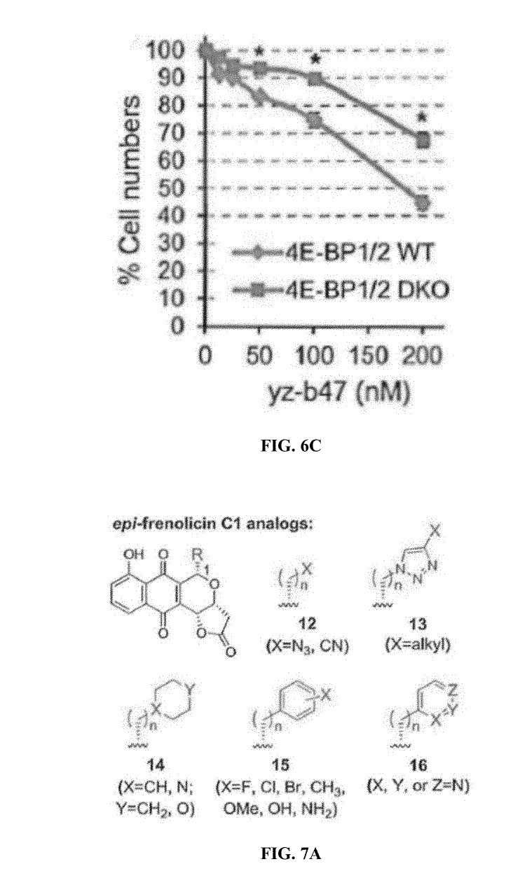

A representative member (b47) from this priority set was further studied in secondary assays as an additional assessment of the prioritization strategy. Specifically, b47 (4-fold more potent than FB against HCT116 but 2-fold less proficient at ROS production, (FIGS. 5B-C) was also found to be 4-fold more potent than FB as an inhibitor of 4E-BP1 phosphorylation (FIG. 6A). Moreover, b47 was also 2-fold less cytotoxic than FB to normal mouse embryo fibroblasts (MEFs) (FIG. 6B) and double knockout of 4E-BP1 and 4E-BP2 (54) in MEFs also reduced the cytotoxic effect of b47 (FIG. 6C).

Epi-FB-Based Analog Synthesis.

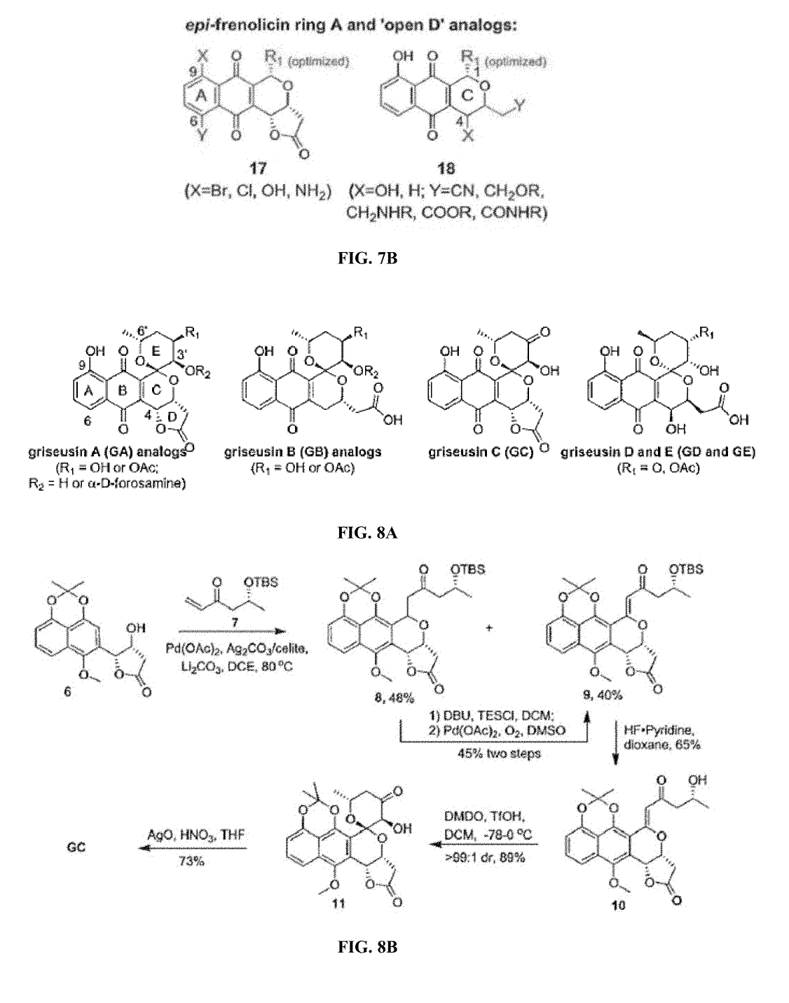

Chemistries directed toward further epi-FB-based analoging are first directed toward further assessment of the impact of C1 variation (FIG. 7A). The optimal C1 substitutions are subsequently evaluated in the context of focused ring A variation (FIG. 7B, (17)), the primary basis of which derives from the best C6/C9 substitutions as determined by preliminary studies described. In addition, the impact of modified open D ring analogs (FIG. 7B, (18)) is being pursued. All proposed analogs are anticipated to be accessible using the general strategy highlighted in FIG. 5A from either commercially available precursors or key starting materials available in four or fewer steps from commercially available reagents.

### Example 2—Griseusins

Synthesis and Preliminary Evaluation of Novel Griseusins.

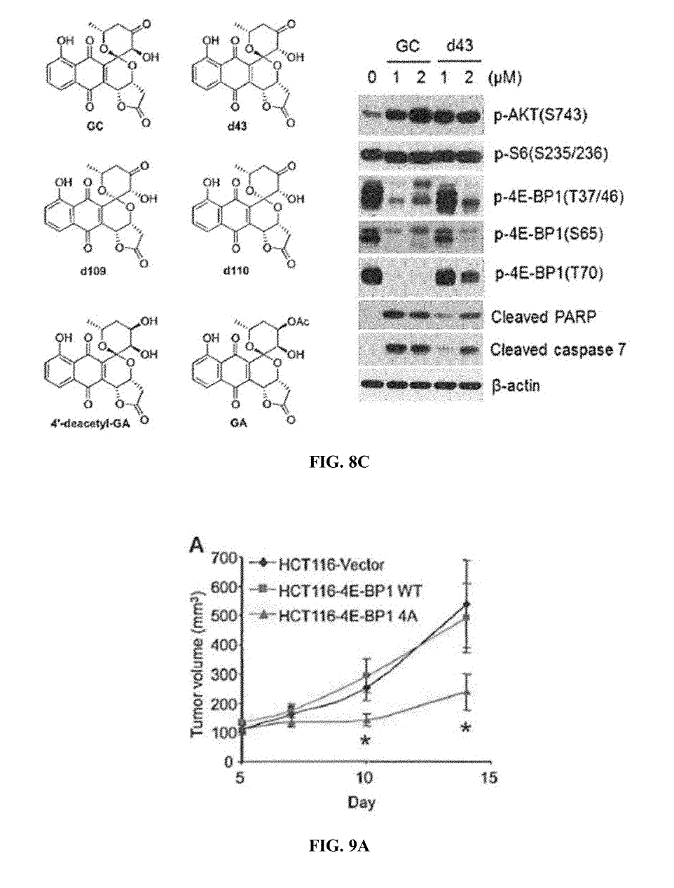

In view of the data above, SAR was further expand with a particular emphasis on C1 modification of the C ring. Within this context, the griseucins (FIG. 8A) isolated from Streptomyces griseus and Nocardiopsis sp., are structurally-related pyranonapthoquinones that notably contain an additional fused C1 spiro-ring system (ring E). This new grisuesin ring E is further elaborated in some members via oxidation, acetylation and/or glycosylation and, like the naturally-occurring frenolicin UCF76-A, some griseusin members also contain an ‘open’ D-ring ester (55-61). While members of this family have been noted for their potent antibiotic, antifungal, and anticancer activities, it is noteworthy that, like kalafugin, 4-dehydro-deacetylgriseusin A was recently identified as COMPARE-negative, consistent with a potentially novel anticancer mechanism (61). Thus, we recently developed an efficient divergent synthetic strategy to access both naturally-occurring griseusins and non-natural analogs (FIG. 8B).

Importantly, in contrast to prior reported synthetic approaches to the griseusins (62-66), the instant method (which draws conceptually on the inventors previously described work with the frenolicin series) stands as the first truly divergent enantioselective method and the most efficient strategy to date. Six griseusin analogs have been synthesized via this method, two of which have been further evaluated in in vitro assays (FIG. 8C). What is particularly striking from this preliminary analysis is the impact of subtle structural modification upon 4E-BP1 phosphorylation inhibition (FIG. 8C) and corresponding HCT116 cytotoxicity. Specifically, while GC displays notable inhibition of 4E-BP1 phosphorylation and cytotoxicity (GC HCT116 IC₅₀ 127 nM), simple inversion of the E ring C3′ stereochemistry led to a reduction in both (d43 HCT116 IC₅₀ 345 nM). In addition, ROS production by GC was also slightly lower than that of the improved FB analog b47 (FIG. 5B). Notably, this work reveals, for the first time, griseusins as effective inhibitors of 4E BP1 phosphorylation and also highlights an efficient and divergent griseusin synthetic strategy.

Griseusin Analog Synthesis.

Using the established divergent strategy highlighted in FIG. 8B, a focused set of E ring-modified griseusin variants may be pursued, the diversification elements of which may focus upon the C/E ring fusion stereochemistry as well as C3′, C4′ and C6′-substitution/stereochemistry. Further optimization may focus upon integrating key A and/or open D ring variations determined to be advantageous in the context of epi-FB analogs described above within the best griseusin ring E-modified analogs.

### Example 3

Altered redox status is a common feature of many cancers in which deregulation of cell signaling and metabolism by multiple genetic alterations often lead to increased generation of intracellular reactive oxygen species (ROS). Although intracellular ROS elevation contributes to tumor initiation and progression, it is believed that agents capable of increasing intracellular ROS beyond the cellular tolerability threshold may represent a potential selective anticancer therapeutic strategy. Within this context, this Example reports the identification of the molecular target and anticancer mechanism of frenolicin B (FB), a classical, but mechanistically undefined, pyranonaphthoquinone (PNQ) natural product commonly used as an anticoccidial feedstock additive.

Specifically, in this Example, FB is identified as a selective inhibitor of peroxiredoxin 1 (Prx1) and glutaredoxin 3 (Grx3), two antioxidant proteins overexpressed in many cancers that play key roles in maintaining cellular redox status. Inhibition of Prx1 and Grx3 by FB induces a concomitant elevation of intracellular ROS which activates the peroxisome-bound tuberous sclerosis complex and thereby inhibits mTORC1-mediated phosphorylation of the translation repressor 4E-BP1, a key effector of the oncogenic activation of the PI3K/AKT/mTOR and RAS/RAF/MEK/ERK pathways in tumorigenesis and metastasis. FB structure-activity relationship (SAR) studies reveal a positive correlation between inhibition of 4E-BP1 phosphorylation, intracellular ROS concentrations, cancer cell cytotoxicity and suppression of tumor growth in vivo. These findings establish FB as the most potent and novel Prx1/Grx3 inhibitor reported to date and also notably highlights 4E-BP1 phosphorylation status as a potential new predictive marker in response to oxidative stress-based therapies in cancer.

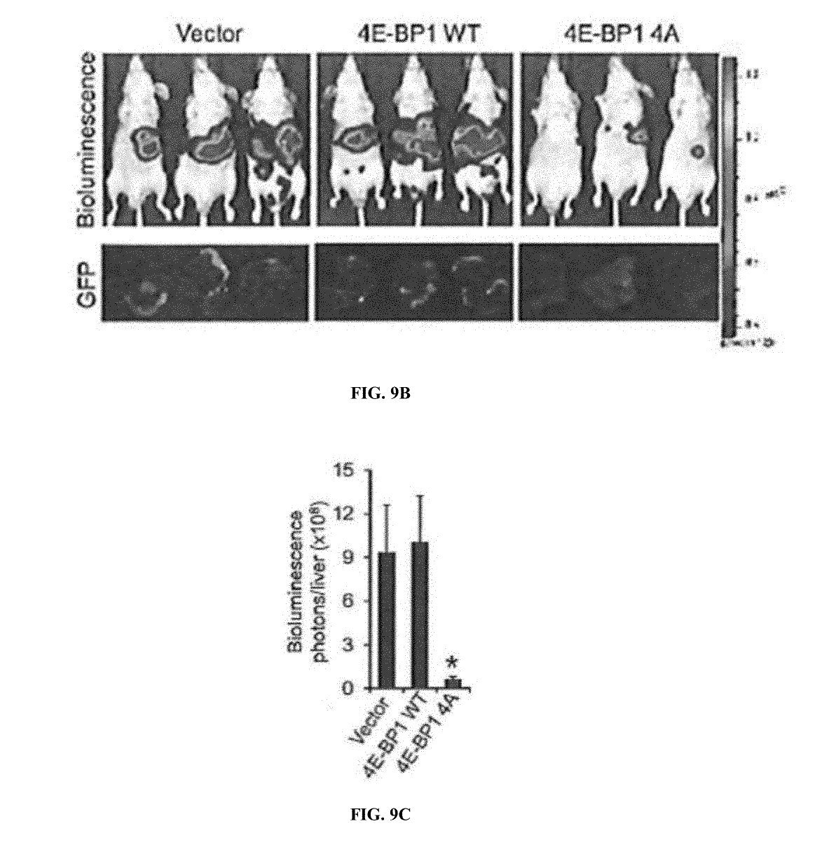

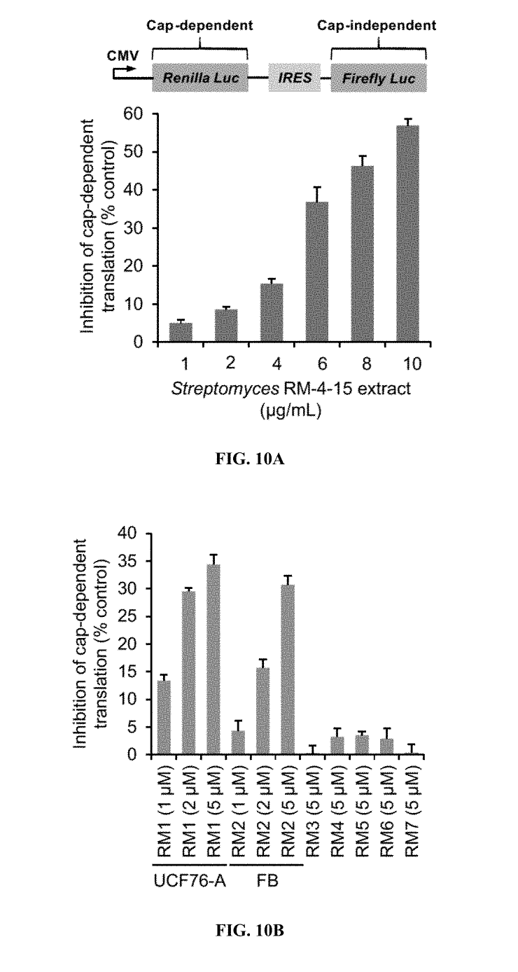

Dysregulation of cap-dependent translation through redundant phosphorylation of the translational repressor 4E-BP1 by multiple oncogenic pathways, such as PI3K/AKT/mTOR and RAS/RAF/MEK/ERK, is associated with malignant progression and therapeutic resistance. To determine the importance of 4E-BP1 dephosphorylation in the repression of cap-dependent translation and tumor progression, a non-phosphorylated 4E-BP1 mutant, 4E-BP1-4A, was generated. The four known phosphorylation sites, T37, T46, S65, and T70 (FIG. 2), are inactivated via alanine replacement. This 4E-BP1 mutant cannot be phosphorylated, binds constitutively to elF4E, and inhibits elF4E-initiated cap-dependent translation (13). As compared to wild-type (WT) 4E-BP1 and vector control, expression of 4E-BP1-4A profoundly suppressed tumor growth and liver metastasis in the HCT116 CRC model (13, 14) (FIGS. 9A-C).

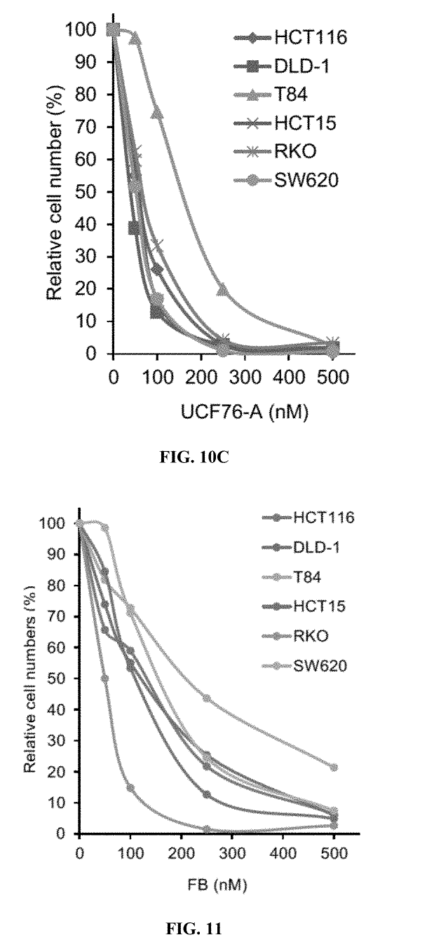

Thus, targeting cap-dependent translation may overcome intra-tumor heterogeneity-mediated resistance and provide a promising strategy for improving cancer therapy. To identify microbial nature products (NPs) capable of targeting cap-dependent translation, crude extracts of prioritized microbes from the Ruth Mullins underground coal mine fire site¹⁴ were screened using a cap-dependent translation-based luciferase reporter assay⁵. This initial screen revealed that extracts of Streptomyces sp. RM-4-15, a strain previously identified to produce a series of known and novel pyranonaphthoquinones (PNQs)¹⁵, contain compounds capable of inhibiting cap-dependent translation (FIG. 10A). Identical assays with purified metabolites from Streptomyces sp. RM-4-15 showed that FB and the related PNQ metabolite UCF76-A could effectively inhibit cap-dependent translation (FIG. 10B). In addition, FB and UCF76-A exhibited potent cytotoxicity against a panel of colorectal cancer (CRC) cell lines (FIGS. 10C and 11). These initial studies highlighted a previously unknown function of PNQs as potent inhibitors of cap-dependent translation.

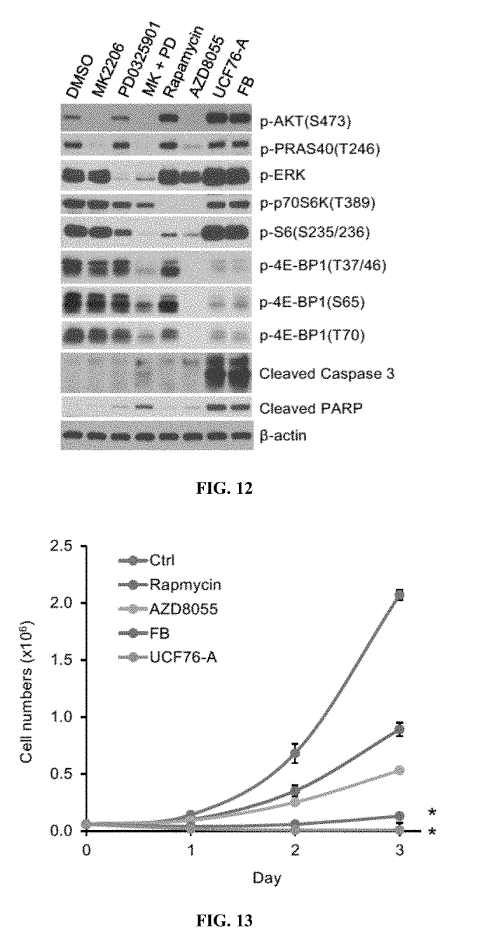

To further probe the function of PNQs within the context of cap-dependent translation, the ability of FB and UCF76-A to modulate 4E-BP1 and p70S6 kinase phosphorylation was compared to that of representative mTOR inhibitors. The mTOR kinase complex 1 (mTORC1), a downstream target of both AKT and ERK signaling, is a well-characterized activator of cap-dependent translation through phosphorylation of 4E-BP1 and p70S6 kinase¹⁷. Rapamycin is an allosteric inhibitor of mTORC1 and can effectively inhibit p70S6K phosphorylation, but only weakly inhibits 4E-BP1 phosphorylation¹⁸, while second generation ATP-competitive mTOR kinase inhibitors such as AZD8055 that inhibit both mTORC1 and mTOR complex 2 (mTORC2) are more effective than rapamycin in inhibiting 4E-BP1 phosphorylation. Like AZD8055 but distinct from rapamycin, FB and UCF76-A effectively inhibited 4E-BP1 phosphorylation in HCT116 CRC cells (FIG. 12).

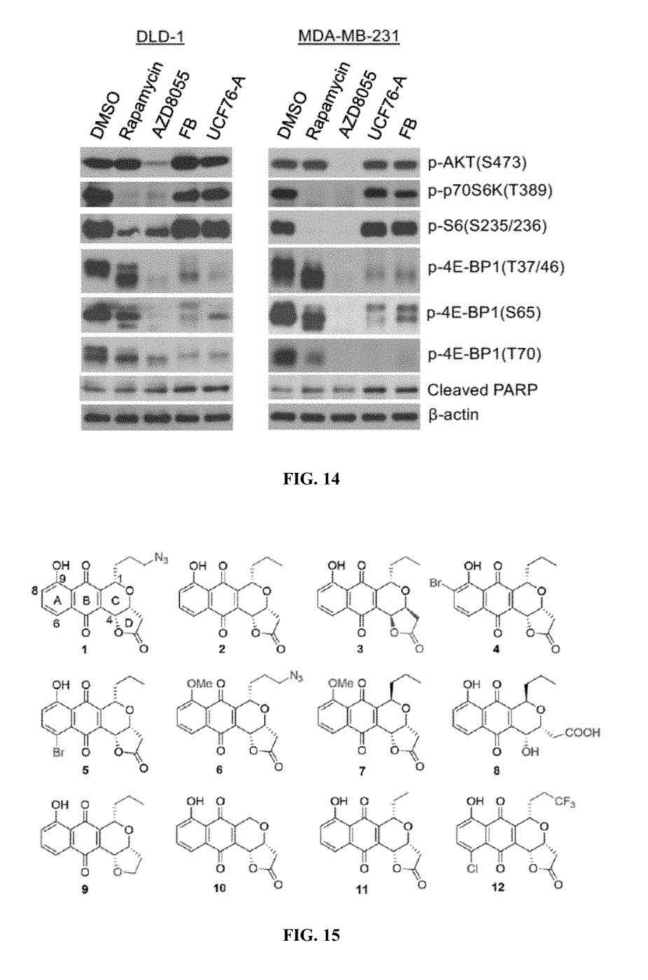

Both rapamycin and AZD8055 potently inhibited phosphorylation of p70S6K and its substrate, the ribosomal protein S6, and AZD8055 also inhibited phosphorylation of the mTORC2 substrate AKT¹⁷. In contrast, FB or UCF76-A did not inhibit S6 or AKT phosphorylation, only weakly inhibited p70S6K phosphorylation, but were potent activators of caspase 3 with induction of the apoptotic marker cleaved PARP and dramatic suppression of HCT116 cell growth (FIGS. 12 and 13). While FB was previously reported to inhibit AKT activity in vitro, no detectable inhibition of AKT phosphorylation or that of its substrate PRAS40 was observed in HCT116 cells treated with FB or UCF76-A (FIG. 12). In addition, effective inhibition of AKT phosphorylation by the highly selective pan-AKT-1/2/3 inhibitor MK2206²¹ led to negligible modulation of 4E-BP1 phosphorylation (FIG. 12), consistent with our previous findings that simultaneous inhibition of both AKT (MK2206) and MEK/ERK (PD0325901) signaling is required to inhibit 4E-BP1 phosphorylation (FIG. 12) and repress cap-dependent translation in CRC cells⁵. Similar effects by FB and UCF76-A were also observed in other CRC (DLD-1) and breast (MDA-MB-231) cancer cell lines (FIG. 14). Furthermore, Invitrogen SelectScreen® Kinase Profiling also revealed no effect on mTOR kinase activity by representative PNQs (data not shown). Collectively, these data suggested that the inhibition of 4E-BP1 phosphorylation by PNQ-based NPs is mechanistically distinct from that of known mTOR, AKT and/or MEK/ERK inhibitors.

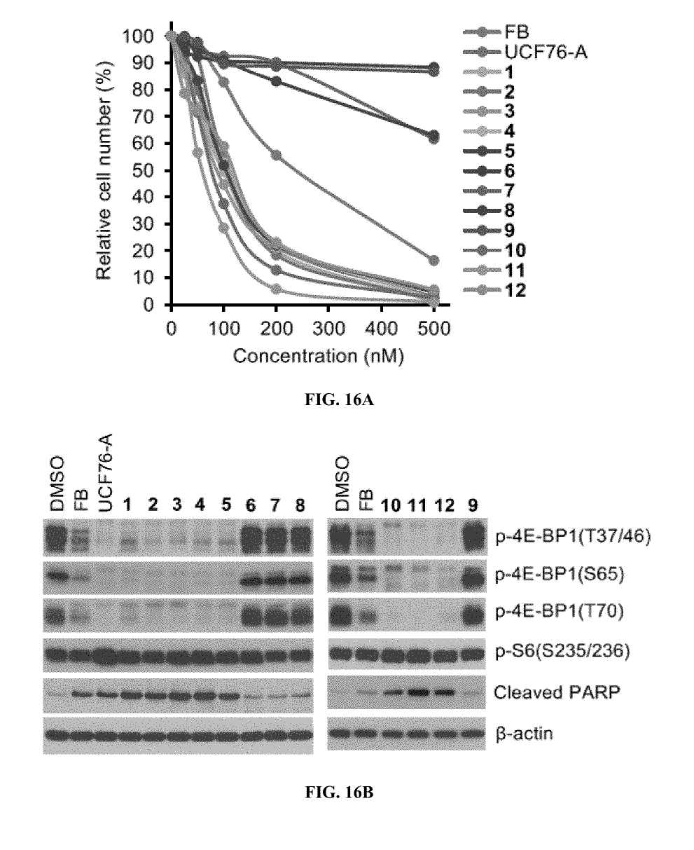

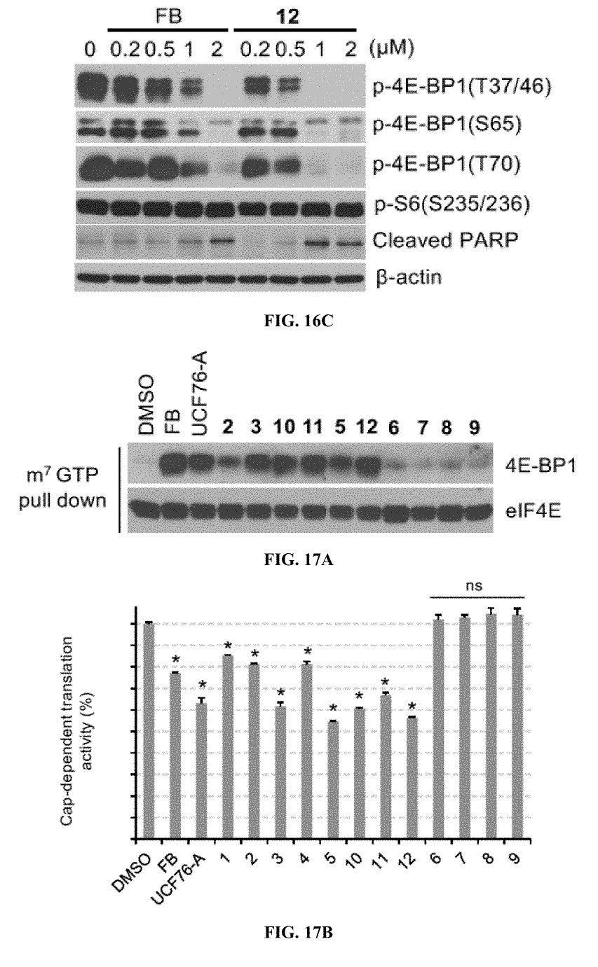

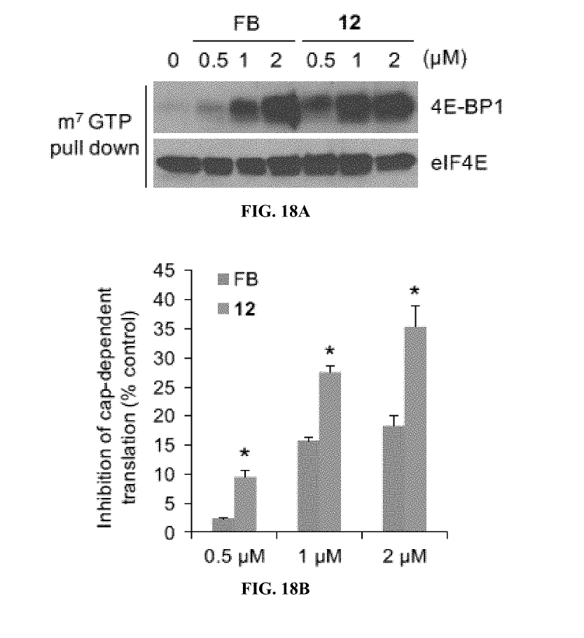

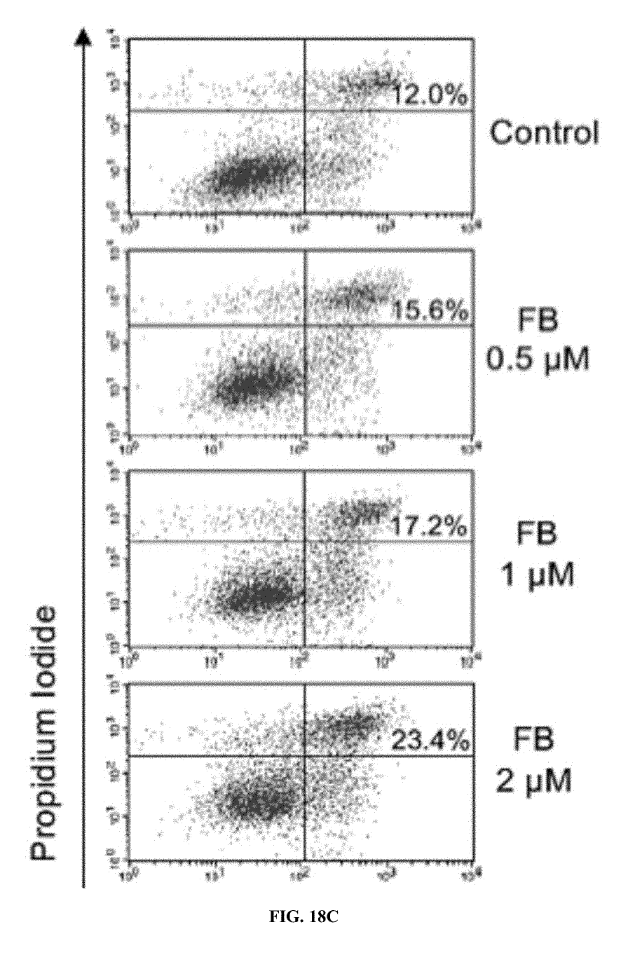

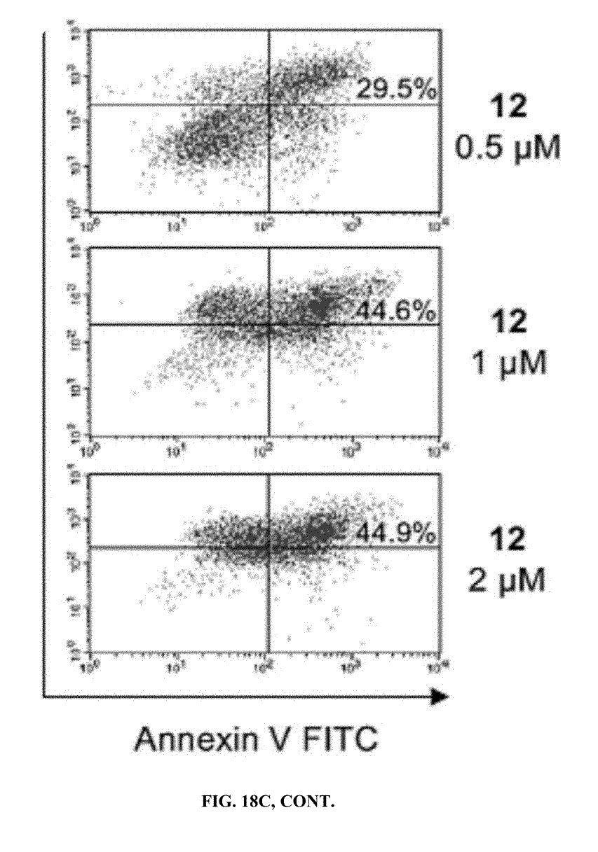

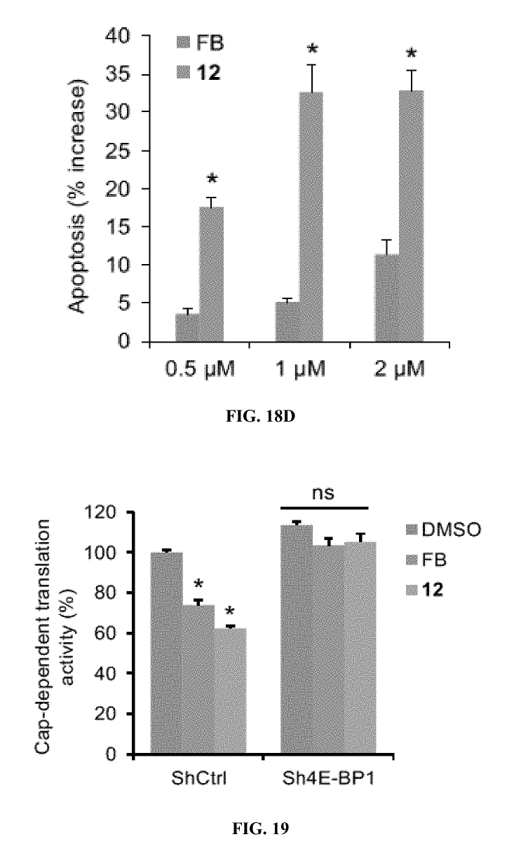

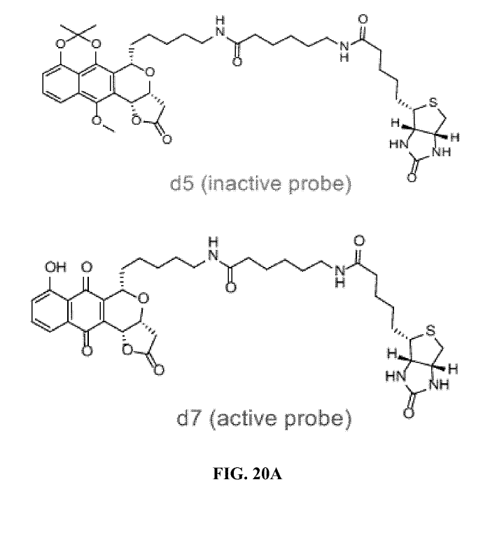

Prompted by the potential mechanistic novelty of PNQs, we evaluated additional FB-based PNQ synthetic analogs (FIG. 15) for CRC cell cytotoxicity (FIG. 16A) and 4E-BP1 phosphorylation inhibitory potential (FIG. 16B). Enabled by our recently reported divergent FB synthetic strategy²², preliminary SAR analysis highlighted a clear correlation between cancer cell cytotoxicity, induction of cleaved PARP, and inhibition of 4E-BP1 phosphorylation (FIGS. 16A-B) where improvements of up to 4-fold in potency over FB were observed (e.g., 12, FIG. 16C). Consistent with ability of dephosphophorylated 4E-BP1 to bind to the eIF4E-mRNA cap complex and suppress cap-dependent translation, active analogs displayed similar effects (FIGS. 17A-B) as exemplified by the correlation of increased inhibition of 4E-BP1 phosphorylation and cap-dependent translation (FIGS. 16C and 18A-B) with improved anti-HCT116 potency and enhanced induction of cell death (FIGS. 18C-D) with 12. Notably, silencing 4E-BP1 gene expression in HCT116 cancer cells as generated previously⁶ almost completely rescued the inhibition on cap-dependent translation activity by FB or 12 (FIG. 19). These data further established the central role of 4E-BP1 phosphorylation in response to PNQ-mediated cancer cell death.

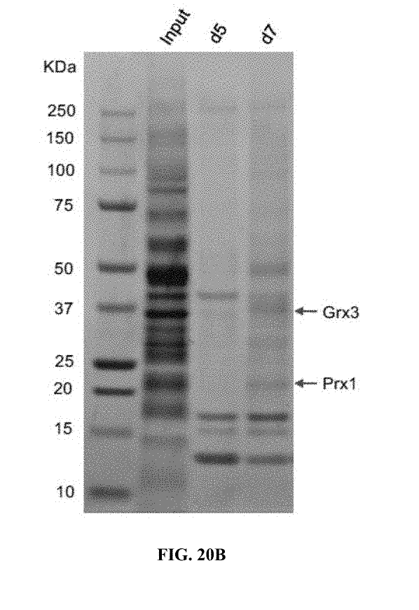

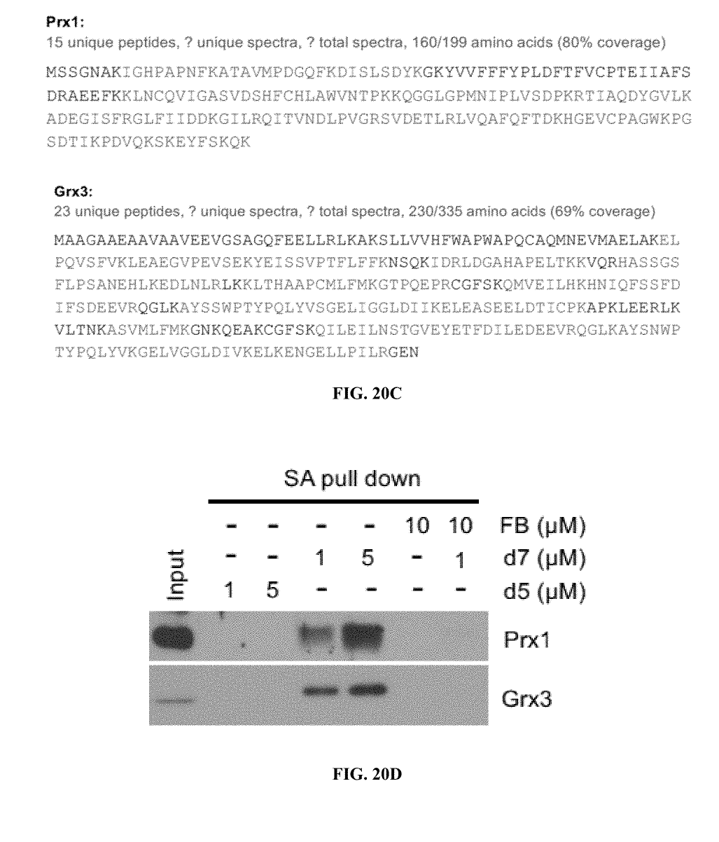

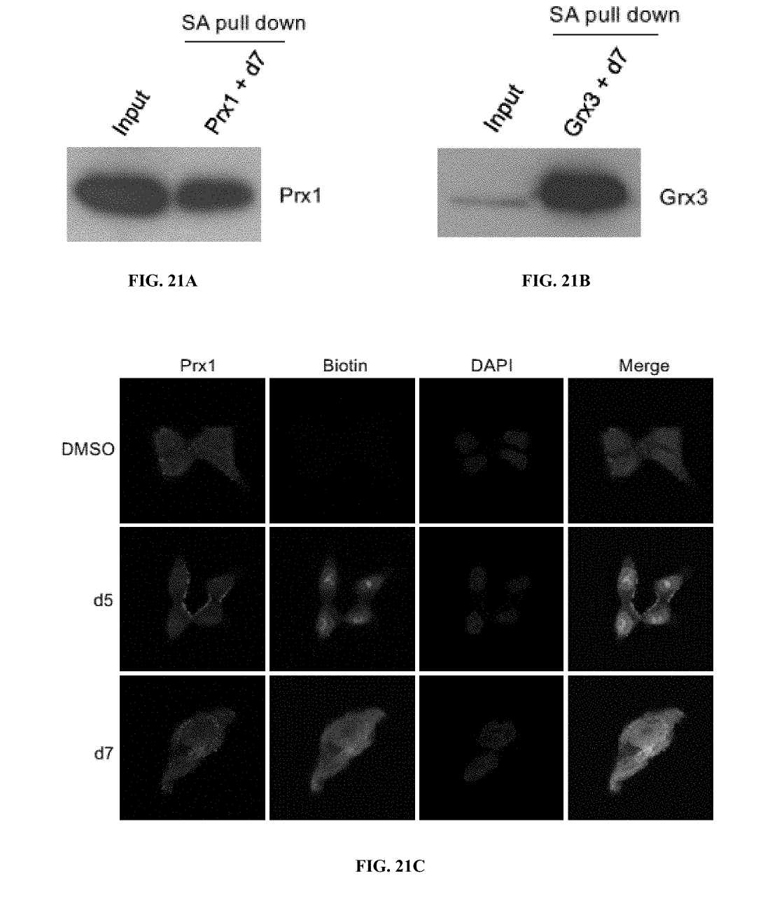

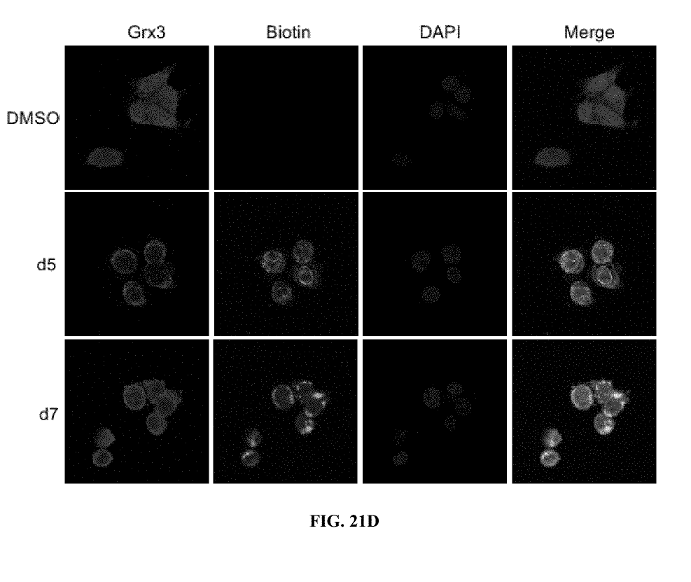

To identify the PNQ molecular target(s) responsible for the observed inhibition of 4E-BP1 phosphorylation and cancer cell cytotoxicity, a comparative affinity pulldown-based target identification strategy was employed. Specifically, guided by the SAR studies described, two FB-based biotinylated probes were synthesized. ‘Active’ probe 1 (d7) retained FB-like activity (inhibition of 4EBP1 phosphorylation and CRC cell line cytotoxicity), while the corresponding activities of structurally-related ‘inactive’ comparator probe 2 (d5) were notably suppressed (FIG. 20A). Parallel incubation of probes 1 and 2 with the Hela cell lysates followed by comparative affinity pulldown and mass spectrometry-based proteomics analysis revealed peroxiredoxin 1 (Prx1) (SEQ ID NO: 1) and glutaredoxin 3 (Grx3) (SEQ ID NO: 2) as principle targets of FB (FIGS. 20B-C). The putative FB-Prx1 and FB-Grx3 interaction was also confirmed using HCT116 cellular extracts and pure Prx1 and Grx3 via a ligand-binding competition assay (FIGS. 20D and 21A-B). Immunofluorescence staining with an antibody against Prx1 or Grx3 and a biotin antibody also revealed colocalization of probe 1 with Prx1 and Grx3 in the cytoplasm and nucleus of HCT116 cells (FIGS. 21C-D).

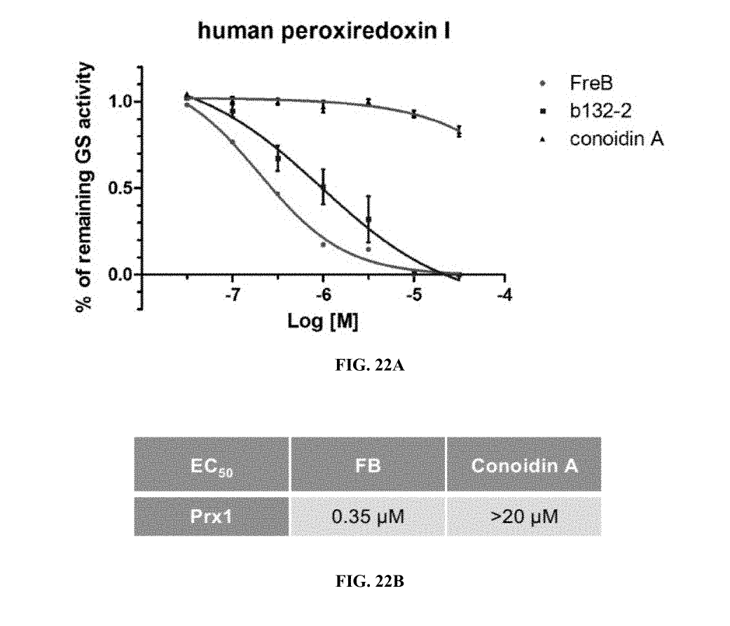

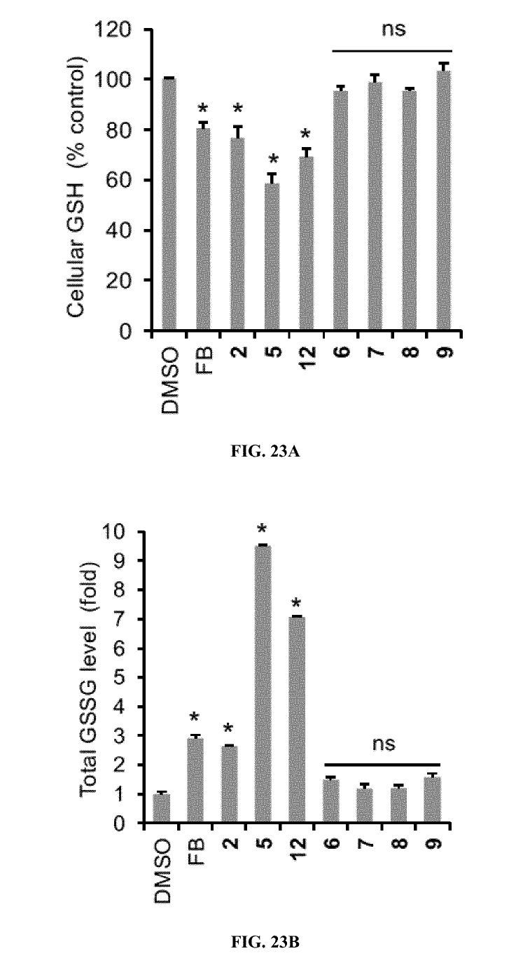

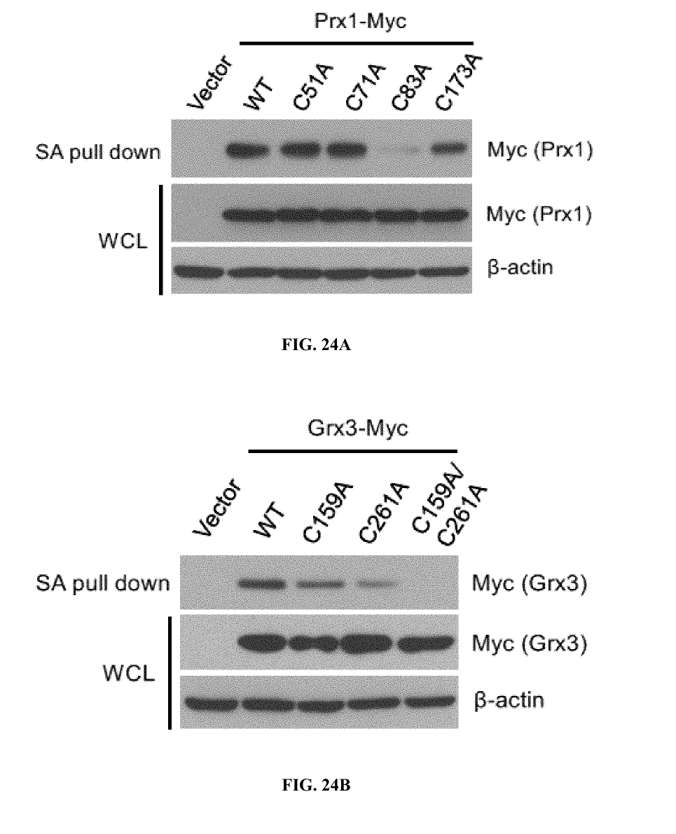

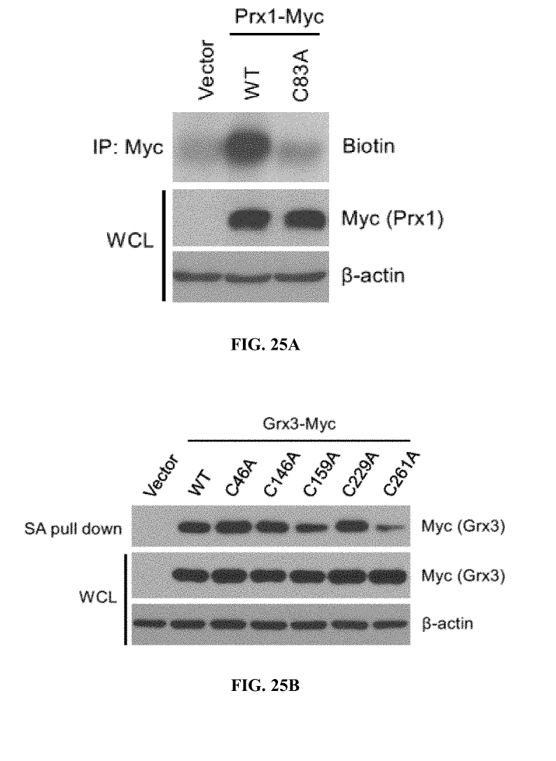

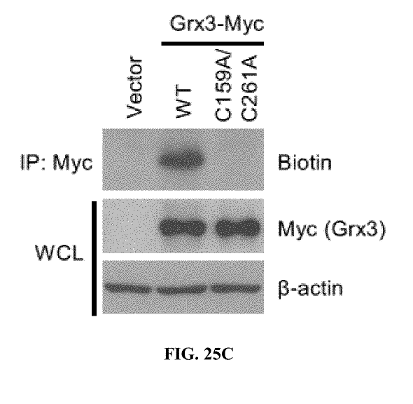

Prx1 and Grx3 are antioxidant enzymes known to regulate oxidative stress. Prx1 catalyzes peroxide reduction of H₂O₂, whereas Grx3 functions as glutathione (GSH)-dependent oxidoreductase by participating in the conversion of reduced GSH to oxidized glutathione disulfide (GSSG). Biochemical inhibition studies using recombination Prx1 revealed FB or semi-synthetic 12 as potent inhibitors of Prx1 with observed K_{iS} >60-fold lower than the most potent Prx1 inhibitor, conoidin A, reported to date (FIGS. 22A-B) and >XX-fold lower than the recently reported Prx1 inhibitor adenanthin. While Grx3 biochemical assays are lacking, FB and active analogs could lead to a decrease in GSH levels and an increase in GSSG levels in HCT116 cells (FIGS. 23A-B). As antioxidant enzymes utilize cysteine-containing active sites to reduce various cellular peroxide or disulfide substrates, the role of Prx1/Grx3 cysteines in FB ligand-binding was assessed through replacing their cysteines to alanines. These studies showed that Prx1 on Cys83 and Grx3 on both Cys159 and Cys261 are essential to FB ligand-binding based on probe 1 affinity capture or immunoprecipitation of Prx1 or Grx3 (FIGS. 24A-B and 25A-C). Finally, mass spectrometry analysis confirmed the covalent modification of Prx1 and Grx3 by FB at these specific target sites. These cumulative studies provide strong validation of Prx1 and Grx3 as the predominate molecular targets of PNQs in cancer cells.

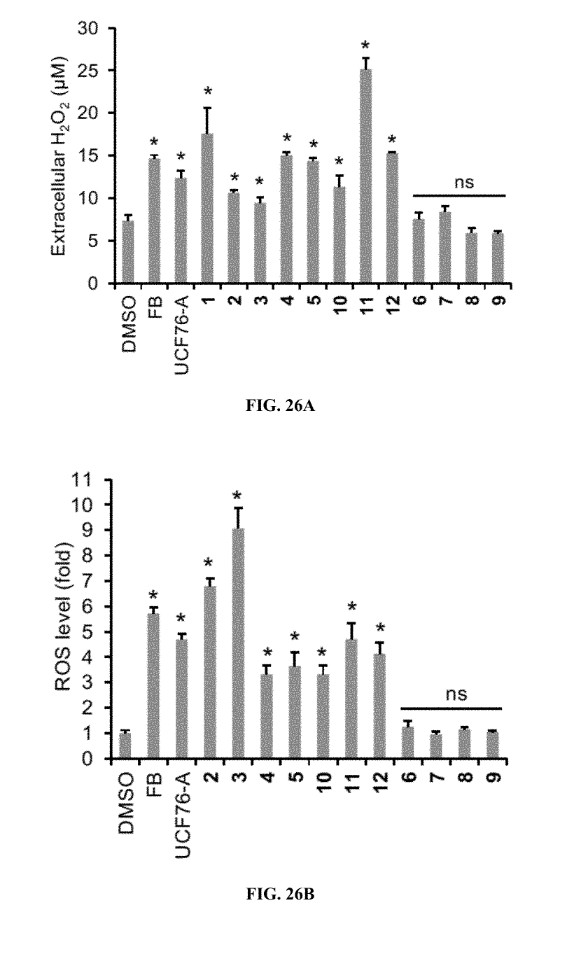

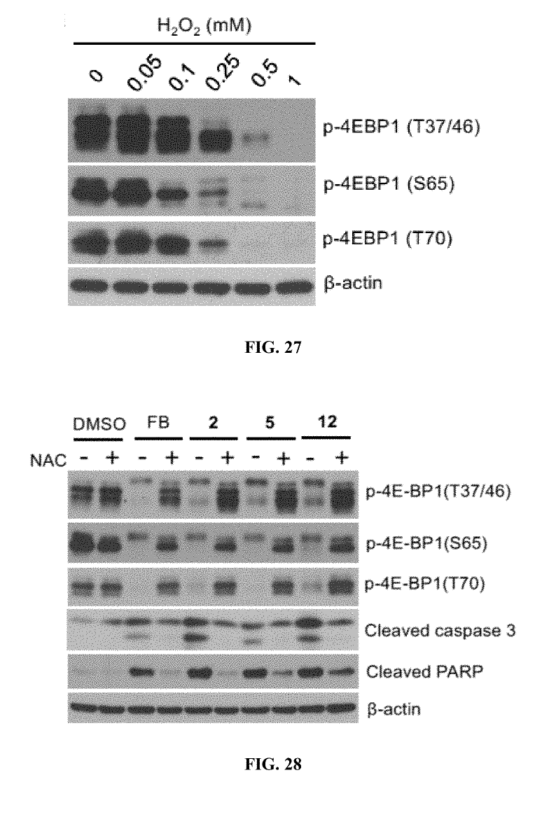

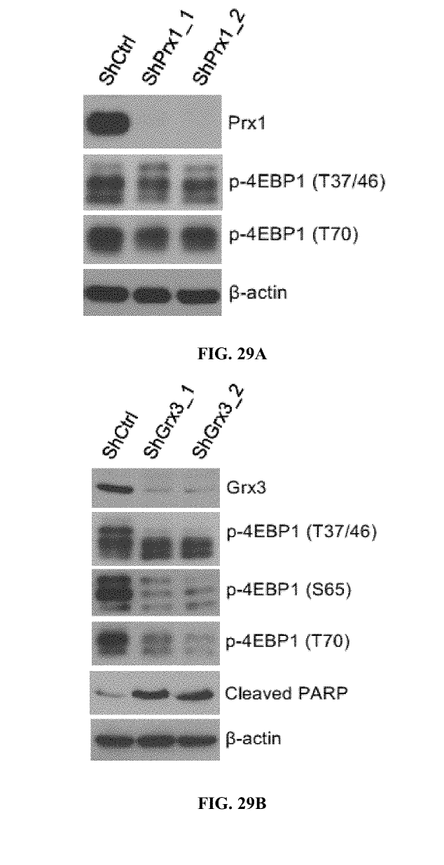

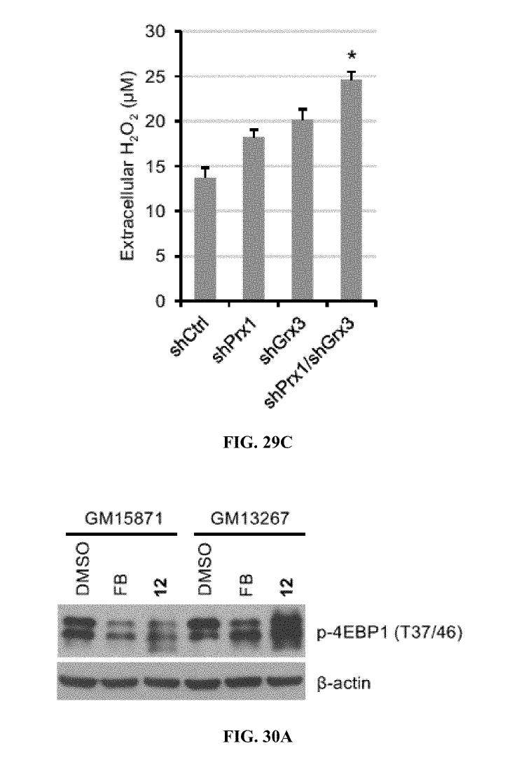

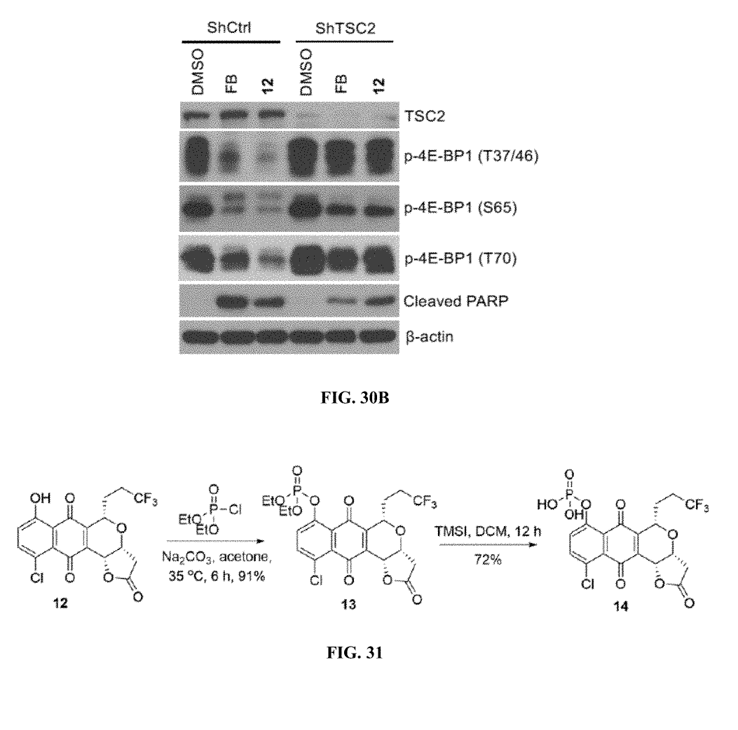

Consistent with Prx1 and Grx3 as the molecular targets, it was found that FB and active surrogates (FIGS. 15 and 16A-B) induce a marked increase in cellular H₂O₂ and ROS (FIGS. 26A-B). Reminiscent of the effects observed with FB treatment, direct treatment of HCT116 cells with H₂O₂ led to a concentration-dependent inhibition of 4E-BP1 phosphorylation and induction of cleaved PARP (FIG. 27) but these effects induced by FB and active analogs were completely abrogated by pretreatment with N-acetyl-L-cysteine, which blocked FB or its analogs-induced ROS (FIG. 28). Similarly, silencing Prx1 or Grx3 expression also increased cellular H₂O₂ levels and led to a concomitant suppression of 4E-BP1 phosphorylation and induction of cleaved PARP, whereas these effects were further enhanced when both Prx1 and Grx3 expression were silenced (FIGS. 29A-C). ROS has been reported to inhibit mTORC1 signaling by activating peroxisome-bound tuberous sclerosis complex (TSC){Zhang, 2013 #1450}. Consistent with this, inhibition of 4E-BP1 phosphorylation and induction of cleaved PARP by FB or 12 were largely prevented in peroxisome-deficient human Zellweger (GM13629) fibroblasts and TSC2-knockdown HCT116 cells (FIGS. 30A-B). Together, these data indicate that PNQs target both Prx1 and Grx3 to increase H₂O₂ or ROS level that is essential for inhibition of mTORC1-mediated 4E-BP1 phosphorylation correlated with induction of cell death.

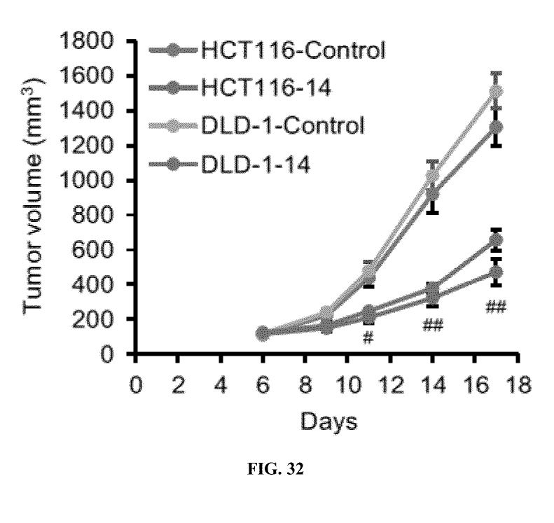

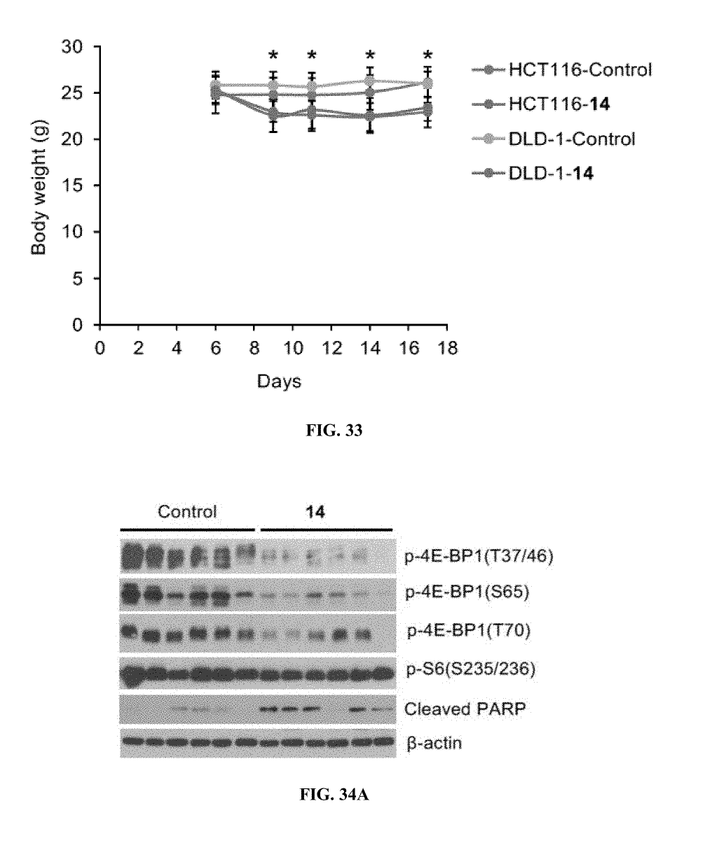

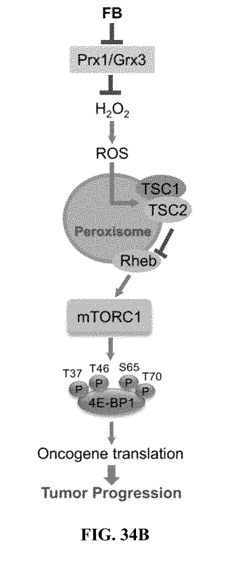

To assess whether this unique anticancer mechanism translates to in vivo efficacy, we utilized an aqueous soluble phosphate prodrug 14 synthesized from 12 in two steps (65% overall yield, FIG. 31). Nude mice bearing established HCT116 or DLD-1 colon cancer xenografts were treated with 14 at a predetermined maximum tolerated dose, 14 mg/kg, daily for five days a week or saline control for 2 weeks. Administration of 14 was able to suppress overall tumor progression and caused 56% (DLD-1) or 64% (HCT116) tumor reduction without significant weight loss (FIGS. 32-33). Western blot analysis of tumor extracts revealed inhibition of 4E-BP1 phosphorylation associated with induction of cleaved PARP by 14 (FIG. 34A). These findings highlight the potential effectiveness of 14 in suitable animal model for CRC and, consistent with the in vitro studies, implicate the inhibition of 4E-BP1 phosphorylation as a contributor to the mechanism of action (FIG. 34B).

FB is the prototypical PNQ-based NP first reported in the late 60's²³ and has since been demonstrated to function as an effective anticoccidial and antimalarial²⁴, the fundamental mechanism(s) for which remain undetermined. The current study highlights, for the first time, that FB and PNQ-based analogs function as inhibitors of Prx1 and Grx3 for cancer therapy by targeting ROS stress-response pathway in which 4E-BP1 phosphorylation functions as a major sensor to guide for the development of this new class of agents and predict their antitumor efficacy. Overexpression of Prx1 and Grx3 often occurs in a variety of cancers, and is associated with redox adaptation that promotes tumor progression and resistance to many anticancer agents and radiation. The use of agents such as PNQs as identified here to abrogate the adaptation mechanism due to the increased intracellular antioxidant capacity in combination with conventional chemotherapy, radiotherapy or target therapy could be an attractive new approach to improve therapeutic outcomes.

Methods

Methods and any associated references are available in the online version of the paper.

Chemistry.

Frenolicin B and UCF76-A were isolated from Streptomyces sp. RM-4-15 as previously described. The syntheses of 1-14 followed previously reported strategies²² and are detailed below. Compound purity for all studies was ≧95% based on HPLC and all compound stock solutions were standardized to reference standards based on HPLC and UV-vis.

General Chemistry Method.

¹H (400 MHz) and ¹³C (100 MHz) NMR spectra were recorded on a Varian Unity Inova 400 MHz instrument (Palo Alto, Calif.). The chemical shifts were reported in δ (ppm) using the δ 7.26 signal of CDCl₃, δ 1.94 signal of CD₃CN and δ 2.50 signal of DMSO-d₆ (¹H NMR), the δ 77.16 signal of CDCl₃, δ 1.32 signal of CD₃CN and δ 39.52 signal of DMSO-d₆ (¹³C NMR) as internal standards. The following abbreviations were used to explain the multiplicities: s=singlet, d=doublet, t=triplet, q=quartet, m=multiplet. HR-ESI-MS experiments were carried out using AB SCIEX TripleTOF® 5600 System. HPLC analyses were performed using an Agilent 1260 system equipped with a DAD detector and a Phenomenex C18 column (4.6×150 mm, 0.5 μm). Semi-preparative/preparative HPLC separation was performed using a Varian Prostar 210 HPLC system equipped with a PDA detector 330 using a Supelco C18 column (25×21.2 mm, 10 μm; flow rate, 10 mL/min). Enantiomeric excess was determined by HPLC with a Chiralpak IC column, compared with racemic isomer. All commercially available reagents were used without further purification, purchased from Sigma-Aldrich, TCI America and Alfa-Aesar. The progress of the reactions was monitored by analytical thin-layer chromatography (TLC) from EMD Chemicals Inc. (Darmstadt, Germany) with fluorescence F254 indicator. And Silica gel (230-400 mesh) for column chromatography was purchased from Silicycle (Quebec City, Canada).

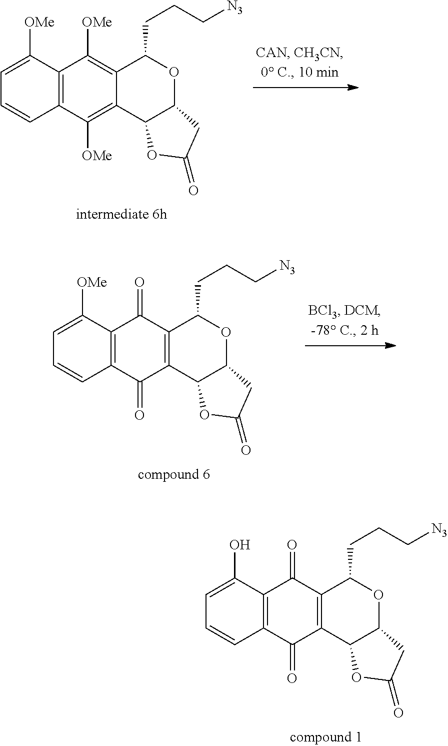 [SMILES: COc1c2c(c(OC)c3c(OC)cccc13)[C@H](CCCN=[N+]=[N-])O[C@@H]1CC(=O)O[C@H]21 ; COc1cccc2c1C(=O)C1=C(C2=O)[C@H]2OC(=O)C[C@H]2O[C@H]1CCCN=[N+]=[N-] ; [N-]=[N+]=NCCC[C@@H]1O[C@@H]2CC(=O)O[C@@H]2C2=C1C(=O)c1c(O)cccc1C2=O]

### (3aR,5S,11bR)-5-(3-Azidopropyl)-7-methoxy-3,3a,5,11b-tetrahydro-2H-benzo[g]furo[3,2-c]isochromene-2,6,11-trione (6)

To a solution of (3aR,5S,11bR)-5-(3-azidopropyl)-6, 7,11-trimethoxy-3,3a,5,11b-tetrahydro-2H-benzo[g]furo[3,2-c]isochromen-2-one (reported intermediate 6 h¹) (41 mg, 0.1 mmol) in a mixture of water (0.5 mL) and acetonitrile (1 mL) at 0° C., a solution of cerium ammonium nitrate (126 mg, 0.2 mmol) in H₂O (0.5 mL) was added in dropwise fashion with stirring. The reaction mixture was stirred for 10 min before the addition of water (5 mL). The mixture was extracted with EtOAc (10 mL×2) and the combined organic layers were washed with brine, dried over Na₂SO₄, filtered, and concentrated. The residue was purified on silica gel using hexane/EtOAc (1/1) to afford 6 as a yellow solid (31 mg, 80% yield). ¹H NMR (400 MHz, CDCl₃): δ=7.77-7.69 (m, 2H), 7.32 (d, J=8.0 Hz, 1H), 5.30 (s, 1H), 4.77 (d, J=9.6 Hz, 1H), 4.34 (d, J=7.2 Hz, 1H), 4.01 (s, 3H), 3.27 (t, J=6.4 Hz, 2H), 2.91 (dd, J=4.4, 17.6 Hz, 1H), 2.73 (d, J=17.6 Hz, 1H), 2.17-2.13 (m, 1H), 1.99-1.92 (m, 1H), 1.76-1.65 (m, 2H); ¹³C NMR (100 MHz, CDCl₃) δ=183.4, 182.3, 174.5, 159.6, 151.3, 135.6, 133.7, 133.3, 120.3, 119.5, 118.3, 72.1, 71.3, 69.7, 56.7, 51.3, 37.4, 30.6, 24.7 ppm; HRMS (ESI) m/z [M+H]⁺ calcd for C₁₉H₁₈N₃O₆ 384.1196, found 384.1199.

### (3aR,5S,11bR)-5-(3-Azidopropyl)-7-hydroxy-3,3a,5,11b-tetrahydro-2H-benzo[g]furo[3,2-c]isochromene-2,6,11-trione (1)

Compound 6 (25 mg, 0.065 mmol) was dissolved in CH₂Cl₂ (1 mL) and then cooled to −78° C. under argon. A solution of BCl₃ (0.1 mL, 0.1 mmol, 1 N in CH₂Cl₂) was added to the mixture with stirring for 2 hours at −78° C. After quenching with saturated aqueous NH₄Cl solution (1 mL), the reaction was diluted with H₂O (5 mL) and EtOAc (5 mL). The organic layer was washed with brine, dried over Na₂SO₄, filtered, and concentrated. The residue was purified on silica gel using hexane/EtOAc (2/1) to afford 1 as an orange solid (19 mg, 80% yield). ¹H NMR (400 MHz, CDCl₃): δ=11.71 (s, 1H), 7.71-7.66 (m, 2H), 7.31 (dd, J=2.4, 7.2 Hz, 1H), 5.28 (t, J=2.0 Hz, 1H), 4.80-4.79 (m, 1H), 4.35 (dd, J=2.4, 4.4 Hz, 1H), 3.30 (t, J=6.4 Hz, 2H), 2.88 (dd, J=4.4, 17.6 Hz, 1H), 2.75 (d, J=17.6 Hz, 1H), 2.27-2.25 (m, 1H), 2.05-2.03 (m, 1H), 1.75-1.72 (m, 1H), 1.64-1.62 (m, 1H); ¹³C NMR (100 MHz, CDCl₃) δ=188.6, 181.4, 174.2, 161.9, 148.9, 137.3, 136.7, 131.4, 125.1, 119.9, 115.1, 71.6, 71.1, 69.7, 51.2, 37.3, 31.1, 24.5 ppm; HRMS (ESI) m/z [M+H]⁺ calcd for C₁₈H₁₆N₃O₆ 370.1039, found 370.1041.

### Epi-Frenolicin B (2)

The synthesis and characterization of 2 were previously reported.

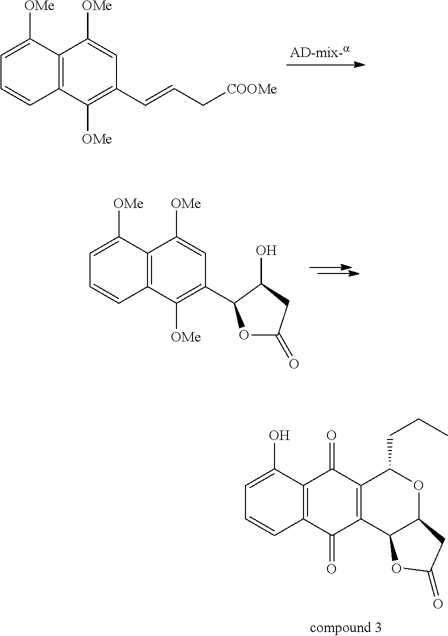 [SMILES: COC(=O)C/C=C/c1cc(OC)c2c(OC)cccc2c1OC ; COc1c([C@@H]2OC(=O)C[C@@H]2O)cc(OC)c2c(OC)cccc12 ; CCC[C@@H]1O[C@H]2CC(=O)O[C@H]2C2=C1C(=O)c1c(O)cccc1C2=O]

### (3aS,5S,11bS)-7-Hydroxy-5-propyl-3,3a,5,11b-tetrahydro-2H-benzo[g]furo[3,2-c]isochromene-2,6,11-trione (3)

This compound was synthesized according to the reported procedures¹ except that AD-mix-α was employed to prepare the enantiomer of reported intermediate 5.^{1 1}H NMR (400 MHz, CDCl₃): δ=11.85 (s, 1H), 7.71-7.65 (m, 2H), 7.30 (dd, J=2.0, 8.0 Hz, 1H), 5.25 (t, J=3.2 Hz, 1H), 4.91 (dd, J=3.2, 10.4 Hz, 1H), 4.62 (dd, J=2.8, 5.2 Hz, 1H), 2.96 (dd, J=5.2, 17.6 Hz, 1H), 2.70 (d, J=17.6 Hz, 1H), 1.71-1.64 (m, 6H), 1.03 (t, J=7.2 Hz, 3H); ¹³C NMR (100 MHz, CDCl₃) δ=188.2, 181.6, 174.0, 162.0, 149.4, 137.3, 135.3, 131.6, 124.9, 119.8, 114.9, 69.7, 68.8, 66.3, 36.9, 33.8, 19.6, 13.6 ppm; HRMS (ESI) m/z [M+H]⁺ calcd for C₁₈H₁₇O₆ 329.1025, found 329.1025.

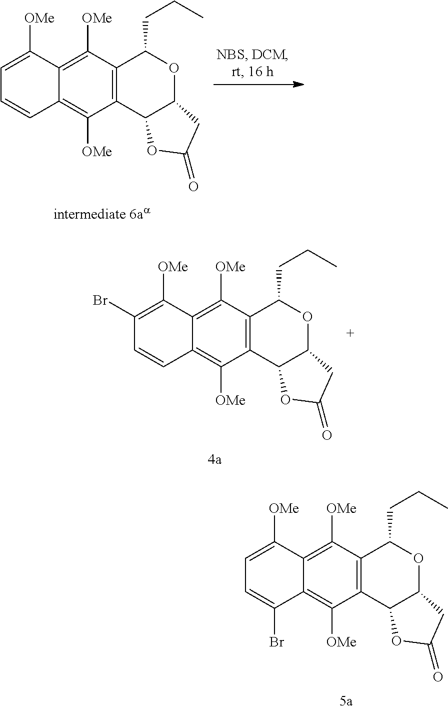 [SMILES: CCC[C@@H]1O[C@@H]2CC(=O)O[C@@H]2c2c1c(OC)c1c(OC)cccc1c2OC ; CCC[C@@H]1O[C@@H]2CC(=O)O[C@@H]2c2c1c(OC)c1c(OC)c(Br)ccc1c2OC ; CCC[C@@H]1O[C@@H]2CC(=O)O[C@@H]2c2c1c(OC)c1c(OC)ccc(Br)c1c2OC]

### (3aR,5S,11bR)-8-Bromo-6,7,11-trimethoxy-5-propyl-3,3a,5,11b-tetrahydro-2H-benzo[g]furo[3,2-c]isochromen-2-one (4a) and (3aR,5S,11bR)-10-bromo-6,7,11-trimethoxy-5-propyl-3,3a,5,11b-tetrahydro-2H-benzo[g]furo[3,2-c]isochromen-2-one (5a)

NBS (53 mg, 0.3 mmol) was added by portion to (3aR,5S,11bR)-6,7,11-trimethoxy-5-propyl-3,3a,5,11b-tetrahydro-2H-benzo[g]furo[3,2-c]isochromen-2-one (reported intermediate 6aα¹) (111 mg, 0.3 mmol) in CH₂Cl₂ (3 mL) at room temperature and the resulting mixture was stirred overnight. After evaporating the volatiles, the residue was purified on silica gel using hexane/EtOAc (6/1) to afford the 4a (40 mg, 29%) and 5a (90 mg, 65%).

### (3aR,5S,11bR)-8-Bromo-6,7,11-trimethoxy-5-propyl-3,3a,5,11b-tetrahydro-2H-benzo[g]furo[3,2-c]isochromen-2-one (4a)

¹H NMR (400 MHz, CDCl₃): δ=7.75 (d, J=8.0 Hz, 1H), 7.64 (d, J=8.0 Hz, 1H), 5.57 (s, 1H), 5.05 (d, J=6.0 Hz, 1H), 4.35 (s, 1H), 4.09 (s, 3H), 3.88 (s, 3H), 3.74 (s, 3H), 2.91 (dd, J=4.0, 18.4 Hz, 1H), 2.77 (d, J=17.6 Hz, 1H), 2.15-2.13 (m, 1H), 2.03-1.99 (m, 1H), 1.76-1.65 (m, 2H), 0.98 (t, J=7.2 Hz, 3H); ¹³C NMR (100 MHz, CDCl₃) δ=175.9, 153.7, 152.5, 147.7, 130.7, 129.8, 124.8, 120.2, 117.5, 107.3, 107.0, 73.2, 73.0, 71.1, 65.0, 62.2, 56.4, 38.5, 37.4, 18.4, 14.1 ppm.

### (3aR,5S,11bR)-10-Bromo-6,7,11-trimethoxy-5-propyl-3,3a,5,11b-tetrahydro-2H-benzo[g]furo[3,2-c]isochromen-2-one (5a)

¹H NMR (400 MHz, CDCl₃): δ=7.72 (d, J=8.0 Hz, 1H), 6.74 (d, J=8.0 Hz, 1H), 5.60 (s, 1H), 5.05 (d, J=6.0 Hz, 1H), 4.34 (s, 1H), 4.11 (s, 3H), 3.99 (s, 3H), 3.96 (s, 3H), 2.90 (d, J=18.4 Hz, 1H), 2.77 (d, J=17.6 Hz, 1H), 2.15-2.13 (m, 1H), 1.99-1.96 (m, 1H), 1.76-1.66k (m, 2H), 0.91 (t, J=7.2 Hz, 3H); ¹³C NMR (100 MHz, CDCl₃) δ=175.9, 156.0, 152.9, 149.5, 133.9, 129.6, 127.1, 126.7, 122.1, 107.8, 107.0, 73.1, 72.8, 71.1, 65.8, 61.8, 56.7, 38.7, 37.8, 18.5, 14.0 ppm.

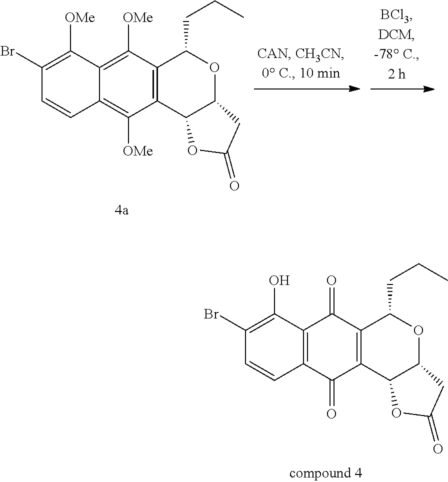 [SMILES: CCC[C@@H]1O[C@@H]2CC(=O)O[C@@H]2c2c1c(OC)c1c(OC)c(Br)ccc1c2OC ; CCC[C@@H]1O[C@@H]2CC(=O)O[C@@H]2C2=C1C(=O)c1c(ccc(Br)c1O)C2=O]

### (3aR,5S,11bR)-8-Bromo-7-hydroxy-5-propyl-3,3a,5,11b-tetrahydro-2H-benzo[g]furo[3,2-c]isochromene-2,6,11-trione (4)

Following the above deprotection protocol for 1, intermediate 4a (40 mg, 0.09 mmol) was used to obtain compound 4 (21 mg, 60% yield). ¹H NMR (400 MHz, CDCl₃): δ=12.38 (s, 1H), 7.95 (d, J=8.0 Hz, 1H), 7.56 (d, J=8.0 Hz, 1H), 5.26 (t, J=2.0 Hz, 1H), 4.77-4.75 (m, 1H), 4.33 (dd, J=2.4, 4.4 Hz, 1H), 2.90 (dd, J=4.4, 17.6 Hz, 1H), 2.74 (d, J=17.6 Hz, 1H), 2.00-1.90 (m, 2H), 1.44-1.42 (m, 1H), 1.28-1.25 (m, 1H), 0.90 (t, J=7.2 Hz, 3H); ¹³C NMR (100 MHz, CDCl₃) δ=188.8, 180.9, 174.4, 158.3, 149.6, 140.4, 136.7, 130.4, 120.1, 119.9, 115.5, 72.0, 71.0, 69.7, 37.4, 36.0, 18.4, 14.1 ppm; HRMS (ESI) m/z [M+H]⁺ calcd for C₁₈H₁₆BrO₆ 407.0130, found 407.0117.

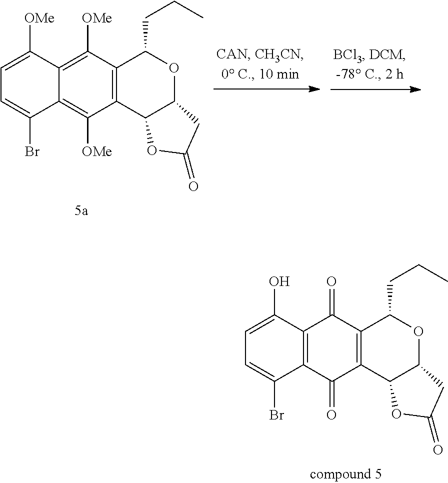 [SMILES: CCC[C@@H]1O[C@@H]2CC(=O)O[C@@H]2c2c1c(OC)c1c(OC)ccc(Br)c1c2OC ; CCC[C@@H]1O[C@@H]2CC(=O)O[C@@H]2C2=C1C(=O)c1c(O)ccc(Br)c1C2=O]

### (3aR,5S,11bR)-10-Bromo-7-hydroxy-5-propyl-3,3a,5,11b-tetrahydro-2H-benzo[g]furo[3,2-c]isochromene-2,6,11-trione (5)

Following the above deprotection protocol for 1, intermediate 5a (90 mg, 0.2 mmol) was used to obtain compound 5 (43 mg, 53% yield). ¹H NMR (400 MHz, CDCl₃): δ=12.28 (s, 1H), 7.83 (d, J=9.2 Hz, 1H), 7.11 (d, J=9.2 Hz, 1H), 5.33 (t, J=2.0 Hz, 1H), 4.75-4.73 (m, 1H), 4.37 (dd, J=2.4, 4.4 Hz, 1H), 2.94 (dd, J=4.4, 17.6 Hz, 1H), 2.73 (d, J=17.6 Hz, 1H), 2.03-2.00 (m, 1H), 1.89-1.85 (m, 1H), 1.44-1.42 (m, 1H), 1.29-1.27 (m, 1H), 0.88 (t, J=7.2 Hz, 3H); ¹³C NMR (100 MHz, CDCl₃) δ=187.9, 180.0, 174.4, 162.0, 148.6, 143.9, 136.9, 128.2, 125.6, 116.5, 113.7, 71.6, 71.1, 69.5, 37.2, 35.7, 18.4, 14.0 ppm; HRMS (ESI) m/z [M+H]⁺ calcd for C₁₈H₁₆BrO₆ 407.0130, found 407.0118.

### 9-Methyl-frenolicin B (7)

The synthesis of 7 was previously reported.^{1 1}H NMR (400 MHz, CDCl₃): δ=7.81 (dd, J=1.2, 8.0 Hz, 1H), 7.72 (t, J=8.0 Hz, 1H), 7.32 (dd, J=1.2, 8.4 Hz, 1H), 5.25 (d, J=2.8 Hz, 1H), 4.87 (m, dd, J=3.2, 10.8 Hz, 1H), 4.60 (dd, J=3.2, 5.2 Hz, 1H), 4.03 (s, 3H), 2.94 (dd, J=5.2, 17.6 Hz, 1H), 2.69 (d, J=17.6 Hz, 1H), 1.83-1.81 (m, 1H), 1.66-1.57 (m, 3H), 1.00 (t, J=7.2 Hz, 3H); ¹³C NMR (100 MHz, CDCl₃) δ=182.7, 182.3, 174.3, 160.2, 150.6, 135.8, 133.8, 132.6, 119.7, 119.5, 118.3, 70.4, 69.1, 66.3, 56.7, 37.0, 33.7, 19.7, 13.6 ppm; HRMS (ESI) m/z [M+H]⁺ calcd for C₁₉H₁₉O₆ 343.1182, found 343.1180.

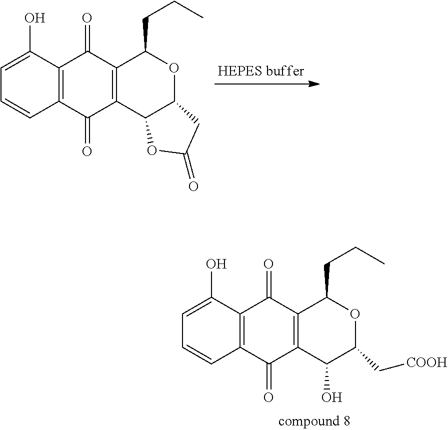 [SMILES: CCC[C@H]1O[C@@H]2CC(=O)O[C@@H]2C2=C1C(=O)c1c(O)cccc1C2=O ; CCC[C@H]1O[C@H](CC(=O)O)[C@H](O)C2=C1C(=O)c1c(O)cccc1C2=O]

### 2-(1R,3R,4R)-4,9-Dihydroxy-5,10-dioxo-1-propyl-3,4,5,10-tetrahydro-1H-benzo[g]isochromen-3-yl)acetic acid (8)

A solution of frenolicin B (10 mg, 0.03 mmol) in DMSO (1 mL) was added to HEPES buffer (8 mL, 50 mM, pH=9.5) in a dropwise fashion at room temperature. The resulting mixture was incubated at 37° C. overnight with brief shaking. After neutralization with 1N HCl to pH=7, the mixture was extracted with Et₂O (10 mL×2). The collected organic layers were washed with brine, dried over Na₂SO₄, concentrated and purified on preparative HPLC (40%-100% CH₃CN/H₂O, 20 min, then 100% CH₃CN, 5 min) to give 8 as a yellow solid (8 mg, 80%). ¹H NMR (400 MHz, CDCl₃): δ=11.91 (s, 1H), 7.65-7.63 (m, 2H), 7.28-7.62 (m, 1H), 4.86 (dd, J=2.0, 10.8 Hz, 1H), 4.69 (d, J=2.4 Hz, 1H), 4.31 (s, 1H), 3.49-3.46 (m, 1H), 2.91-2.89 (m, 2H), 2.65 (s, 1H), 1.74-1.65 (m, 6H), 1.02 (t, J=7.2 Hz, 3H); ¹³C NMR (100 MHz, CDCl₃) δ=189.0, 183.5, 175.4, 161.8, 146.6, 141.0, 136.8, 131.6, 125.0, 119.5, 114.9, 71.0, 67.1, 60.3, 35.4, 33.0, 19.8, 13.7 ppm; HRMS (ESI) m/z [M−H]⁻ calcd for C₁₈H₁₇O₇ 345.0974, found 345.0971.

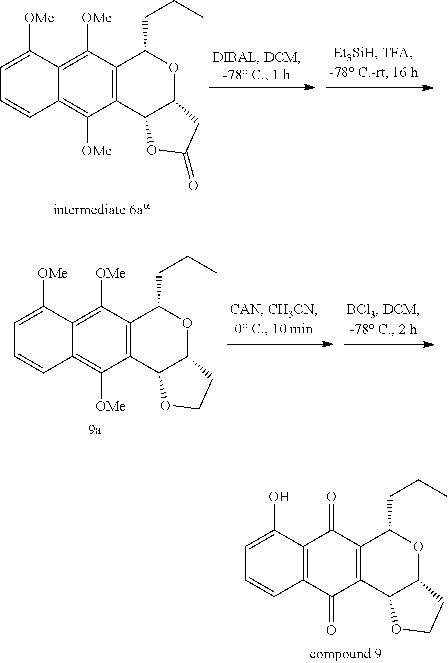 [SMILES: CCC[C@@H]1O[C@@H]2CC(=O)O[C@@H]2c2c1c(OC)c1c(OC)cccc1c2OC ; CCC[C@@H]1O[C@@H]2CCO[C@@H]2c2c1c(OC)c1c(OC)cccc1c2OC ; CCC[C@@H]1O[C@@H]2CCO[C@@H]2C2=C1C(=O)c1c(O)cccc1C2=O]

### (3aR,5S,11bR)-7-hydroxy-5-propyl-3,3a,5,11b-tetrahydro-2H-benzo[g]furo[3,2-c]isochromene-6,11-dione (9)

To a stirred solution of (3aR,5S,11bR)-6,7,11-trimethoxy-5-propyl-3,3a,5,11b-tetrahydro-2H-benzo[g]furo[3,2-c]isochromen-2-one (reported intermediate 6aα¹) (93 mg, 0.25 mmol) in CH₂Cl₂ (2.5 mL) at −78° C. was added DIBAL-H (0.5 mL, 1 M in toluene). The reaction was quenched with saturated aqueous potassium sodium tartrate (1 mL) after 1 hour. The mixture was allowed to warm to room temperature with stirring, extracted with CH₂Cl₂ (10 mL×2), and the combined organics washed with brine, dried over Na₂SO₄, and concentrated. The residue was dissolved in CH₂Cl₂ containing trifluoroacetic acid (58 μL, 0.75 mmol) and cooled to −78° C. to which triethylsilane (119 μL, 0.75 mmol) was added in dropwise fashion. The resulting mixture was allowed to warm to room temperature with stirring overnight. After evaporating the volatiles, the residue was purified on silica gel using hexane/EtOAc (15/1) to obtain the tetrahydrofuran intermediate 9a (65 mg, 73% for two steps) as a colorless oil.

Following the above deprotection protocol for 1, 9a (65 mg, 0.18 mmol) was used to obtain 9 (28 mg, 50% yield). ¹H NMR (400 MHz, CDCl₃): δ=11.84 (s, 1H), 7.68-7.59 (m, 2H), 7.26-7.24 (m, 1H), 4.714.68 (m, 1H), 4.59 (t, J=2.0 Hz, 1H), 4.16-4.10 (m, 3H), 2.24-2.20 (m, 1H), 2.04-1.99 (m, 1H), 1.51-1.46 (m, 1H), 1.32-1.28 (m, 1H), 0.91 (t, J=7.2 Hz, 3H); ¹³C NMR (100 MHz, CDCl₃) δ=189.7, 182.7, 161.5, 148.0, 139.6, 136.7, 131.9, 124.4, 119.4, 115.1, 74.9, 72.2, 70.2, 67.7, 36.1, 33.6, 18.3, 14.2 ppm; HRMS (ESI) m/z [M+H]⁺ calcd for C₁₈H₁₉O₅ 315.1232, found 315.1224.

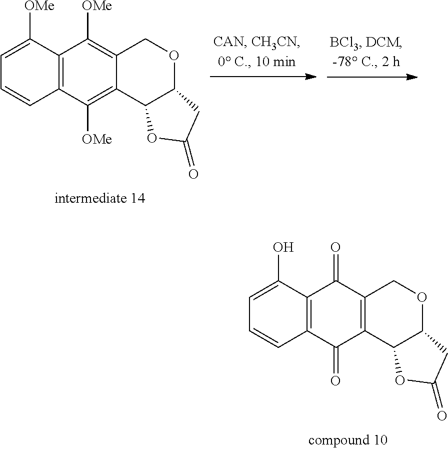 [SMILES: COc1c2c(c(OC)c3c(OC)cccc13)CO[C@@H]1CC(=O)O[C@H]21 ; O=C1C[C@H]2OCC3=C(C(=O)c4cccc(O)c4C3=O)[C@H]2O1]

### (3aR,11bR)-7-Hydroxy-3,3a,5,11b-tetrahydro-2H-benzo[g]furo[3,2-c]isochromene-2,6,11-trione (10)

Following the above deprotection protocol for compound 1, (3aR,11bR)-6,7,11-trimethoxy-3,3a,5,11b-tetrahydro-2H-benzo[g]furo[3,2-c]isochromen-2-one (reported intermediate 14¹) (33 mg, 0.1 mmol) was used to obtain 10 (16 mg, 54% yield). ¹H NMR (400 MHz, CDCl₃): δ=11.71 (s, 1H), 7.70-7.68 (m, 2H), 7.30 (dd, J=2.0, 7.6 Hz, 1H), 5.26 (t, J=2.0 Hz, 1H), 4.95 (d, J=18.8 Hz, 1H), 4.47-4.39 (m, 2H), 2.95 (dd, J=2.0, 17.6 Hz, 1H), 2.76 (d, J=17.6 Hz, 1H); ¹³C NMR (100 MHz, CDCl₃) δ=188.0, 173.8, 161.9, 146.1, 137.5, 137.4, 135.8, 131.6, 124.9, 120.0, 114.7, 72.5, 69.1, 61.4, 37.0 ppm; HRMS (ESI) m/z [M+H]⁺ calcd for C₁₅H₁₁O₆ 287.0556, found 287.0546.

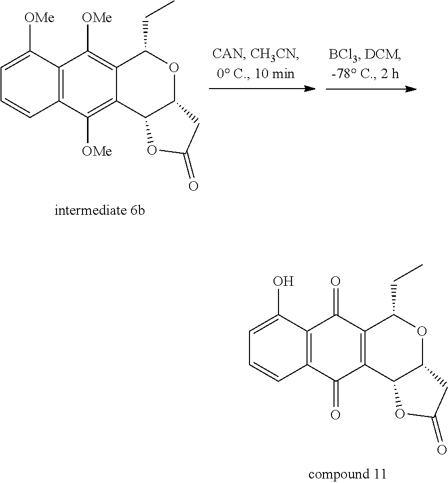 [SMILES: CC[C@@H]1O[C@@H]2CC(=O)O[C@@H]2c2c1c(OC)c1c(OC)cccc1c2OC ; CC[C@@H]1O[C@@H]2CC(=O)O[C@@H]2C2=C1C(=O)c1c(O)cccc1C2=O]

### (3aR,5S,11bR)-5-Ethyl-7-hydroxy-3,3a,5,11b-tetrahydro-2H-benzo[g]furo[3,2-c]isochromene-2,6,11-trione (11)

Following the above deprotection protocol for compound 1, (3aR,5S,11bR)-5-ethyl-6,7,11-trimethoxy-3,3a,5,11b-tetrahydro-2H-benzo[g]furo[3,2-c]isochromen-2-one (reported intermediate 6b¹) (36 mg, 0.1 mmol) was used to obtain 11 (19 mg, 61% yield). ¹H NMR (400 MHz, CDCl₃): δ=11.75 (s, 1H), 7.68-7.66 (m, 2H), 7.30-7.28 (m, 1H), 5.27 (s, 1H), 4.74 (s, 1H), 4.34 (s, 1H), 2.90 (dd, J=4.0, 17.6 Hz, 1H), 2.75 (d, J=17.6 Hz, 1H), 2.14-2.03 (m, 2H), 0.89 (t, J=7.2 Hz, 3H); ¹³C NMR (100 MHz, CDCl₃) δ=188.7, 181.5, 174.5, 161.8, 149.5, 137.2, 136.7, 131.5, 124.9, 119.7, 115.1, 72.8, 70.9, 69.8, 37.4, 27.1, 9.2 ppm; HRMS (ESI) m/z [M+H]⁺ calcd for C₁₇H₁₅O₆ 315.0869, found 315.0857.

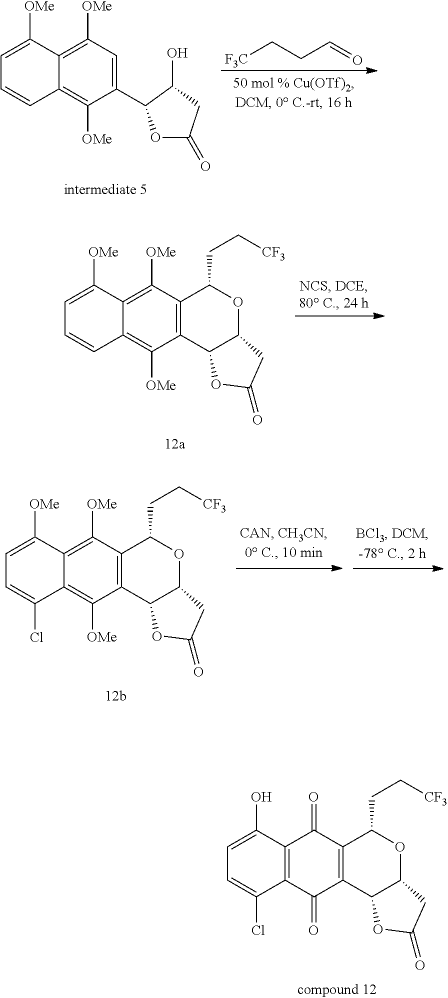 [SMILES: COc1c([C@H]2OC(=O)C[C@H]2O)cc(OC)c2c(OC)cccc12 ; O=CCCC(F)(F)F ; COc1c2c(c(OC)c3c(OC)cccc13)[C@H](CCC(F)(F)F)O[C@@H]1CC(=O)O[C@H]21 ; COc1ccc(Cl)c2c(OC)c3c(c(OC)c12)[C@H](CCC(F)(F)F)O[C@@H]1CC(=O)O[C@H]31 ; O=C1C[C@H]2O[C@@H](CCC(F)(F)F)C3=C(C(=O)c4c(Cl)ccc(O)c4C3=O)[C@H]2O1]

### (3aR,5S,11bR)-10-Chloro-7-hydroxy-5-(3,3,3-trifluoropropyl)-3,3a,5,11b-tetrahydro-2H-benzo[g]furo[3,2-c]isochromene-2,6,11-trione (12)

To a solution of (4R,5R)-4-hydroxy-5-(1,4,5-trimethoxynaphthalen-2-yl)dihydrofuran-2(3H)-one (reported intermediate 5¹) (192 mg, 0.6 mmol) and 4,4,4-trifluorobutanal (152 mg, 1.2 mmol) in anhydrous CH₂Cl₂ at 0° C., Cu(OTf)₂ (108 mg, 0.3 mmol) was added with stirring. The temperature was allowed to rise to room temperature and the mixture was stirred for 16 hours. After evaporating the volatiles, diastereoselectivity of the crude mixture was evaluated via NMR and then purified on silica gel using hexane/EtOAc (3/1-2/1) to give 12a as a colorless solid (200 mg, 81% yield, >20:1 dr ratio). ¹H NMR (400 MHz, CDCl₃): δ=7.73 (dd, J=1.2, 8.4 Hz, 2H), 7.46 (t, J=8.4 Hz, 1H), 6.95 (d, J=7.6 Hz, 1H), 5.58 (d, J=2.4 Hz, 1H), 5.12-5.10 (m, 1H), 4.37 (dd, J=2.4, 4.0 Hz, 1H), 4.09 (s, 3H), 4.02 (s, 3H), 3.75 (s, 3H), 2.92 (dd, J=4.4, 17.2 Hz, 1H), 2.77 (d, J=17.6 Hz, 1H), 2.632.60 (m, 1H), 2.31-2.20 (m, 2H), 2.06-2.02 (m, 1H); ¹³C NMR (100 MHz, CDCl₃) δ=175.5, 156.3, 153.4, 149.6, 130.6, 127.0, 126.2, 126.0, 121.7, 119.4, 115.2, 107.7, 72.9, 71.9, 71.3, 64.6, 61.7, 56.4, 38.3, 29.6 (q), 27.7 ppm.

N-chlorosuccinamide (68 mg, 0.51 mmol) was added to a CH₂Cl₂ solution of 12a (200 mg, (0.47 mmol) at room temperature. The resulting mixture was heated to 80° C. with stirring for 24 hours. After evaporating the volatiles, the residue was purified on silica gel using hexane/EtOAc (5/1) to afford 12b (200 mg, 93% yield). ¹H NMR (400 MHz, CDCl₃): δ=7.48 (d, J=8.4 Hz, 2H), 6.83 (d, J=8.4 Hz, 1H), 5.58 (d, J=2.0 Hz, 1H), 5.12-5.09 (m, 1H), 4.35 (dd, J=2.4, 4.0 Hz, 1H), 3.98 (s, 3H), 3.97 (s, 3H), 3.7 (s, 3H), 2.92 (dd, J=4.4, 17.2 Hz, 1H), 2.76 (d, J=17.6 Hz, 1H), 2.62-2.56 (m, 1H), 2.28-2.24 (m, 2H), 2.06-2.00 (m, 1H); ¹³C NMR (100 MHz, CDCl₃) δ=175.4, 171.2, 155.4, 153.2, 149.9, 130.2, 127.4, 126.8, 123.5, 122.0, 120.7, 107.7, 72.7, 71.6, 71.5, 65.7, 61.8, 56.7, 38.3, 29.5 (q), 28.0 ppm.

Following the above deprotection protocol for compound 1, 12b (200 mg, 0.43 mmol) was used to obtain 12 (120 mg, 67% yield). ¹H NMR (400 MHz, CDCl₃): δ=12.28 (s, 1H), 7.65 (d, J=9.2 Hz, 1H), 7.25 (d, J=9.2 Hz, 1H), 5.35 (t, J=1.6 Hz, 1H), 4.79-4.77 (m, 1H), 4.39 (dd, J=2.8, 4.4 Hz, 1H), 2.96 (dd, J=4.4, 17.6 Hz, 1H), 2.76 (d, J=17.6 Hz, 1H), 2.53-2.48 (m, 1H), 2.20-2.01 (m, 3H); ¹³C NMR (100 MHz, CDCl₃) δ=187.7, 179.6, 173.8, 161.7, 146.6, 141.1, 137.9, 127.3, 126.7, 126.0, 115.9, 71.4, 70.3, 69.2, 37.1, 29.5 (q), 26.3 ppm; HRMS (ESI) m/z [M+NH₄]⁺ calcd for C₁₈H₁₆ClF₃NO₆ 434.0618, found 434.0615.

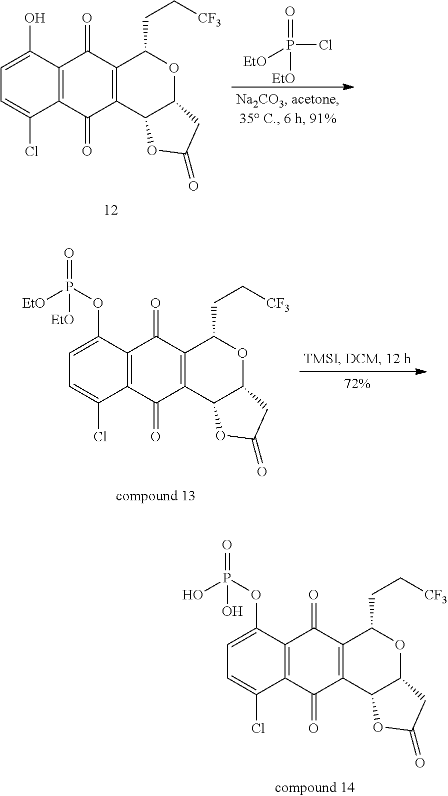 [SMILES: O=C1C[C@H]2O[C@@H](CCC(F)(F)F)C3=C(C(=O)c4c(Cl)ccc(O)c4C3=O)[C@H]2O1 ; CCOP(=O)(Cl)OCC ; CCOP(=O)(OCC)Oc1ccc(Cl)c2c1C(=O)C1=C(C2=O)[C@H]2OC(=O)C[C@H]2O[C@H]1CCC(F)(F)F ; O=C1C[C@H]2O[C@@H](CCC(F)(F)F)C3=C(C(=O)c4c(Cl)ccc(OP(=O)(O)O)c4C3=O)[C@H]2O1]

### (3aR,5S,11bR)-10-Chloro-2,6,11-trioxo-5-(3,3,3-trifluoropropyl)-3,3a,5,6,11,11b-hexahydro-2H-benzo[g]furo[3,2-c]isochromen-7-yl diethyl phosphate (13)

To a solution of 12 (84 mg, 0.2 mmol) and Na₂CO₃ (106 mg, 1 mmol) in acetone (1 mL) was added diethyl chlorophosphate (44 μL, 0.3 mmol). The resulting mixture was stirred at 35° C. for 6 hours. Upon completion, the reaction mixture was directly loaded to silica gel using hexane/EtOAc (1/1) to obtain 13 as a pale yellow liquid (100 mg, 91% yield). ¹H NMR (400 MHz, CDCl₃): δ=7.72 (d, J=9.2 Hz, 1H), 7.66 (d, J=9.2 Hz, 1H), 5.38 (t, J=2.0 Hz, 1H), 4.74-4.72 (m, 1H), 4.37-4.32 (m, 1H), 4.31-4.23 (m, 4H), 2.95 (dd, J=4.8, 17.6 Hz, 1H), 2.74 (d, J=17.6 Hz, 1H), 2.38-2.34 (m, 1H), 2.25-2.23 (m, 2H), 1.99-1.96 (m, 1H), 1.40-1.34 (m, 6H); ¹³C NMR (100 MHz, CDCl₃) δ=182.2, 179.8, 173.8, 148.8, 148.2, 148.1, 138.5, 135.4, 131.3, 129.2, 128.3 (t), 125.7 (t), 71.8, 70.6, 68.7, 65.5 (d), 65.4 (d), 37.0, 29.7 (q), 25.6, 16.2, 16.1 ppm.

### (3aR,5S,11bR)-10-Chloro-2,6,11-trioxo-5-(3,3,3-trifluoropropyl)-3,3a,5,6,11,11b-hexahydro-2H-benzo[g]furo[3,2-c]isochromen-7-yl dihydrogen phosphate (14)

Iodotrimethylsilane (29 μL, 0.2 mmol) was added to a solution of 13 (100 mg, 0.18 mmol) in anhydrous CH₂Cl₂ (360 μL) with stirring under argon. The reaction was stirred at room temperature and monitored by HPLC analysis. Upon completion (typically 8-12 hours) the volatiles were evaporated and the residue was taken up in the mixture of Et₂O (5 mL) and H₂O (5 mL). The aqueous phase was collected and lyophilized to afford 14 as a yellow amorphous powder (64 mg, 72%). ¹H NMR (400 MHz, DMSO-d₆): δ=7.91 (d, J=8.8 Hz, 1H), 7.68 (dd, J=1.2, 9.2 Hz, 1H), 5.37 (t, J=2.0 Hz, 1H), 4.79-4.78 (m, 1H), 4.43 (dd, J=2.8, 4.8 Hz, 1H), 3.21 (dd, J=4.8, 17.6 Hz, 1H), 2.54 (d, J=17.6 Hz, 1H), 2.40-2.24 (m, 3H), 1.93-1.86 (m, 1H); ¹³C NMR (100 MHz, DMSO-d₆) δ=181.7, 180.0, 175.2, 149.1, 148.8, 137.5, 134.8, 128.7, 127.8, 125.8, 125.7, 71.5, 69.7, 69.1, 36.5, 28.4 (q), 24.6 ppm; HRMS (ESI) m/z [M+H]⁺ calcd for C₁₈H₁₄ClF₃O₉P 497.0016, found 497.0008.

Cell Culture and Transfection.

Human colon (HCT116, DLD-1, T84, HCT15, RKO, SW620) and breast (MDA-MB-231) cancer cell lines were obtained from the American Type Culture Collection (ATCC, Manassas, Va.) and cultured in the appropriate medium with supplements as recommended by ATCC. All the cell lines were tested for mycoplasma contamination via PCR (e-Myco Plus kit; iNtRON Biotechnology) and were found to be negative. In addition, all the cell lines are routinely checked for morphologic and growth changes, to probe for cross-contaminated, or genetically drifted cells. If any of these features occur, we use the short tandem repeat (STR) profiling service by ATCC to re-authenticate the cell lines. HCT116 cells with stable knockdown of 4E-BP1 and its control stable transfectants were generated as described previously {Ye, 2014 #1007}. For transient transfection, cells were transiently transfected with DNA using Lipofectamine 3000 according to the manufacturer's protocol (Life Technologies, Carlsbad, Calif.).

Generation of Stable Cells Using Lentiviral Infection.

The lentiviral-based shRNA (pLKO.1 plasmids) used to knock down expression of human Prx1 and Grx3, and the Non-Target Control shRNA (SHC002) were purchased from Sigma (St Louis, Mo.). On the basis of knockdown efficiency of Prx1 and Grx3 protein expression in HCT116 cells, we selected two shPrx1 and two shGrx3 clones for this study. The mature antisense sequences are as follows: 5′-GCTTT CAGTGATAGGGCAGAA-3′ (shPrx1_1) (SEQ ID NO: 3), 5′-GATGAGACTTTGAGACTAGTT-3′ (shPrx1_2) (SEQ ID NO: 4), 5′-CCTACCTATCCTCAGCTCTAT-3′ (shGrx3_1) (SEQ ID NO: 5), 5′-GAACGAAGTTATGGCAGAGTT-3′ (shGrx3_2) (SEQ ID NO: 6). To generate lentivirus-expressing shRNA for Prx1 and Grx3, we transfected 293T cells with pLKO.1-non-silence (for vector control virus), pLKO.1-shPrx1 or pLKO.1-shGrx3 with Lipofectamine 3000 transfection reagent. Twenty-four hours after transfection, the medium was changed, and then it was collected at 24-h intervals. The collected medium containing lentivirus was filtered through 0.45-mm filters. Cells were seeded at 50% confluence 24 h before infection, and the media were replaced with a medium containing lentivirus. After infection for 24 h, the medium was replaced with fresh medium and the infected cells were selected with 2 μg ml⁻¹ puromycin for 7-10 days as described previously {Ye, 2014 #1007}.

Plasmids.

The human Prx1 and Grx3 were cloned into the pCMV6-Entry expression vector with C-terminal Myc-Flag Tag (PS100001, OriGene, Rockville, Md.) for transient transfection. Using the pCMV6-Prx1-Myc-Flag or pCMV6-Grx3-Myc-Flag as a template, the Prx1 mutant (C51A, C71A, C83A, C173A) and Grx3 mutant (C46A, C146A, C159A, C229A, C261A, C159A/C261A) constructs were generated using the QuikChange XLII site-directed mutagenesis kit (Stratagene, La Jolla, Calif.). The primers used are listed in Supplementary Table 1. All constructs were confirmed using enzyme digestion and automated DNA sequencing.

Antibodies and Chemicals.

Antibodies for phospho-Akt (Ser473) (4060), phospho-p70S6 Kinase (Thr389) (9234), phospho-S6 (Ser235/236) (4858), phospho-4E-BP1 (Thr37/46) (2855), phospho-4E-BP1 (Ser65) (13443), phospho-4E-BP1 (Thr70) (13396), 4E-BP1 (9644), eIF4E (2067), Myc-tag (2276, 2278), cleaved caspase-3 (Asp175) (9661) and cleaved PARP (Asp214) (5625) were from Cell Signaling Technology (Danvers, Mass.). Peroxiredoxin 1 antibody (ab15571) was from Abcam (Cambridge, Mass.). PICOT (Grx3, sc-100601) antibody was from Santa Cruz Biotechnology (Dallas, Tex.). Biotin antibody (A150-109A) was from Bethyl Laboratories (Montgomery, Tex.) and β-actin antibody (A5411) was from Sigma. Rapamycin, AZD8055 and MK2206 were obtained from Selleck (Houston, Tex.).

Western Blot Analysis and Immunoprecipitation.

Cells were lysed in NP-40 lysis buffer (50 mM Tris-HCl, pH 7.5, 150 mM NaCl, 1 mM EDTA, 1% NP-40, 10% glycerol, protease and phosphatase inhibitor cocktail). Western blot analysis were performed using equivalent total protein loadings as described previously. For immunoprecipitation, the cell lysates were incubated with the indicated antibody overnight followed by incubation with a 50% slurry of protein G sepharose beads for 3 h at 4° C. The beads were washed three times with the lysis buffer, and the immunoprecipitated protein complexes were resuspended in 2× Laemmli sample buffer followed by western blot analysis.

Cap-Dependent Translation Assay.

Cells (8×10⁴) were transfected with a bicistronic luciferase reporter plasmid (0.2 μg), pcDNA3-rLuc-PolioIRES-fLuc, which directs cap-dependent translation of the Renilla luciferase gene and cap-independent Polio IRES-mediated translation of the firefly luciferase gene. After 24 h transfection, cells were treated with indicated compounds for 12 h, and cell lysates were assayed for Renilla and firefly luciferase activities using a dual-luciferase assay kit (Promega, Madison, Wis.). Cap-dependent Renilla luciferase activity was normalized against cap-independent firefly luciferase activity as the internal control. The ratio of Renilla/firefly luciferase activity was calculated for cap-dependent translational activity as described previously. Each experiment was performed in triplicate and repeated at least three times.

Cap-Binding Assay.

Cap-binding assay was performed as described previously. Briefly, cell lysates (500 μg protein) as prepared in the NP-40 lysis buffer were incubated at 4° C. overnight with m⁷GTP Sepharose beads (GE Healthcare Life Sciences, Pittsburgh, Pa.) to capture eIF4E and its binding partners. Precipitates were washed three times with the lysis buffer and resuspended in 2× Laemmli sample buffer followed by western blot analysis.

Cell Growth and Apoptosis Assays.

Cell growth was assessed as described previously. Briefly, 5×10⁴ cells were seeded in 6-well plates in triplicates. After 24 h, cells were treated with the indicated compounds and incubated at 37° C. The cells were cultured for 3 days and the number of viable cells was counted using the Vi-CELL XR 2.03 (Beckman Coulter, Brea, Calif.). For apoptosis, cells were analyzed by flow cytometry using the Annexin V-FITC Apoptosis Detection Kit according to the manufacturer's protocol (BD Biosciences, San Jose, Calif.).

Pull-Down and MS Analysis of FB-Bound Proteins.

To identify the target protein for FB, FB-based biotinylated active d7 and inactive d5 were synthesized as described in XXXX. HeLa cell pellets were purchased from National Cell Culture Center (Minneapolis, Minn.) and lysed in 20 ml of the NP-40 lysis buffer. The cell lysates (250 mg protein) were pre-cleared with 200 μl streptavidin beads (20353, Thermo Fisher Scientific, Grand Island, N.Y.) at 4° C. for 1 h. Binding reactions were performed by incubating the pre-cleared cell lysates (125 mg proteins/40 ml) with 2 μM d5 or d7 at 4° C. for 3 h, followed by adding 100 μl streptavidin beads and incubating the mixtures overnight at 4° C. After incubation, the beads were washed four times with the lysis buffer, and the bead-bound proteins were eluted, resolved by SDS-PAGE, and visualized by Coomassie blue staining. The protein-containing band in the gel was excised, followed by in-gel digestion and analysis by LC-MS/MS.

Immunofluorescence.

Cells grown on glass bottom culture dishes were incubated with 25 μM d5, 25 μM d7 or DMSO as control for 5 h. After treatment, cells were fixed with 4% paraformaldehyde in PBS for 15 min, permeabilized in 0.2% Triton X-100 and 0.5% BSA in PBS for 5 min and then blocked with 4% BSA in PBS for 10 min. The cells were incubated overnight at 4° C. with the rabbit polyclonal antibody against Prx1 (1:200, ab15571, Abcam) and mouse monoclonal anti-Biotin-FITC (1:200, 200-092-211, Jackson ImmunoResearch, West Grove, Pa.), or with the mouse monoclonal antibody against Grx3 (1:200, MAB7560, R&D Systems, Minneapolis, Minn.) and rabbit polyclonal anti-Biotin-FITC (1:200, ab53469, Abcam). After three washes with 0.05% Triton X-100 in PBS, cells were incubated with anti-rabbit secondary antibody conjugated with Texas-Red for Prx1 (1:500, 111-585-144, Jackson ImmunoResearch) or anti-mouse secondary antibody conjugated with Texas-Red for Grx3 (1:500, 111-585-144, Jackson ImmunoResearch) for 1 h. Cells were washed, mounted with UltraCruz DAPI containing mounting medium (Santa Cruz Biotechnology), viewed, and photographed under a FluoView 1200 confocal microscope (Olympus, Center Valley, Pa.).

Measurement of Cellular ROS and H₂O₂ Production.

The ROS production was determined using the CellROX Deep Red Flow Cytometry Assay Kit according to the manufacturer's protocol (Life Technologies). Briefly, cells were treated with 2 μM of the indicated compounds or DMSO as control for 1 h. After treatment, cells were incubated with 0.5 μM of CellROX Deep Red reagent for 1 h at 37° C., washed twice with PBS and immediately analyzed by a FACScan flow cytometer. For the measurement of cellular H₂O₂ level, cells were treated with 2 μM of the indicated compounds for 5 h.

Cellular Glutathione Assay.

Cells were treated with 2 μM of the indicated compounds or DMSO as control for 5 h. After treatment, a total number of 1×10⁶ cells were lysed in 100 μl of ice-cold NP-40 lysis buffer for 10 min. The lysate was centrifuged for 10 min and the supernatant was used for glutathione assay using the ApoGSH glutathione detection kit according to the manufacturer's protocol (BioVision Research Products, Mountain view, CA). The total amount of GSH was measured using a fluorescence plate reader at Ex./Em.=380/460 nm.

Quantification of Glutathione Disulfide (GSSG).

Cells were treated with 2 μM of the indicated compounds or DMSO as control for 5 h. Quantification of GSSG was essentially performed using the manufactures instructions for a microplate assay for GSH/GSSG (Oxford Biomedical Research, Inc, Oxford, Mich.). A total number of 0.5×10⁶ cells were collected in 1.5 ml centrifuge tubes containing ice-cold buffer with the thiol scavenger to keep GSSG in its oxidized form. The cells were homogenized with a Teflon pestle and the cell suspension sonicated in icy water for 2-3 minutes. Ice-cold metaphosphoric acid was added to deproteinate the samples. The samples were centrifuged at 1000×g at 4° C. and the supernatants were used for determining the GSSG concentration according to the manufactures protocol using a microplate reader with 405 nm filter. The change in GSSG levels in the indicated compound-treated samples was expressed as fold change compared to control (DMSO) treated samples.

Animal Experiments.

Male athymic nude mice (5-6 weeks old) were purchased from Taconic (Hudson, N.Y., USA). Experiments were carried out under a protocol approved by the University of Kentucky Institutional Animal Care and Use Committee. HCT116 and DLD-1 xenograft tumors were established by subcutaneously injecting 3×10⁶ cells in a 1:1 mixture of media and Matrigel (BD Biosciences) into the right flank. For efficacy studies, mice were randomized among control and treated groups (n=8 per group) when tumors were well-established (˜120 mm³). Compound 14 was prepared freshly in saline and administered by intraperitoneal injection at 14 mg/kg once per day, Mon-Fri per week. Control mice received saline solution. Tumor dimensions were measured using a caliper and tumor volumes were calculated as mm³=π/6× larger diameter×(smaller diameter)². Tumors were excised and snap frozen in liquid nitrogen, homogenized in 2% SDS lysis buffer and then processed for Western blot analysis as described previously⁵.

Statistical Analysis.

Results are expressed as the mean±s.e.m. where applicable. A two-tailed Student's t-test was used to compare between groups as outlined in each legend. Differences between groups were considered statistically significant at P<0.05.

All patents, patent applications, publications, and other published materials mentioned in this specification, unless noted otherwise, are herein incorporated by reference to the same extent as if each individual publication, patent, or patent application was specifically and individually indicated to be incorporated by reference, including the references set forth in the following list:

### REFERENCES

1. Harvey, A. L. et al. The re-emergence of natural products for drug discovery in the genomics era. Nat. Rev. Drug. Discov. 14, 111-129 (2015).
2. Cragg, G. M. et al. New horizons for old drugs and drug leads. J. Nat. Prod. 77, 703-723 (2014).
3. Wurth, R. et al. Drug-repositioning opportunities for cancer therapy: Novel molecular targets for known compounds. Drug Discov. Today 21, 190-199 (2016).
4. Bhat, M. et al. Targeting the translation machinery in cancer. Nat. Rev. Drug Discov. 14, 261-278 (2015).
5. She, Q.-B. et al. 4E-BP1 is a key effector of the oncogenic activation of the AKT and ERK signaling pathways that integrates their function in tumors. Cancer Cell 18, 39-51 (2010).
6. Ye, Q. et al. ERK and AKT signaling cooperate to translationally regulate survivin expression for metastatic progression of colorectal cancer. Oncogene 33, 1828-1839 (2014).
7. Cai, W. et al. Loss of 4E-BP1 function induces EMT and promotes cancer cell migration and invasion via cap-dependent translational activation of snail. Oncotarget 5, 6015-6027 (2014).
8. Mi, W. et al. AKT inhibition overcomes rapamycin resistance by enhancing the repressive function of PRAS40 on mTORC1/4E-BP1 axis. Oncotarget 6, 13962-13977 (2015).
9. Avdulov, S. et al. Activation of translation complex eIF4F is essential for the genesis and maintenance of the malignant phenotype in human mammary epithelial cells. Cancer Cell 5, 553-563 (2004).
10. Hsieh, A. C. et al. Genetic dissection of the oncogenic mTOR pathway reveals druggable addiction to translational control via 4EBP-eIF4E. Cancer Cell 17, 249-261 (2010).
11. Hsieh, A. C. et al. The translational landscape of mTOR signalling steers cancer initiation and metastasis. Nature 485, 55-61 (2012).
12. Ducker, G. S. et al. Incomplete inhibition of phosphorylation of 4E-BP1 as a mechanism of primary resistance to ATP-competitive mTOR inhibitors. Oncogene 33, 1590-1600 (2014).
13. Zhang, Y. & Zheng, X. F. mTOR-independent 4E-BP1 phosphorylation is associated with cancer resistance to mTOR kinase inhibitors. Cell Cycle 11, 594-603 (2012).
14. O'Keefe, J. M. K. et al. CO, CO2, and Hg emission rates from the Truman Shepherd and Ruth Mullins coal fires, Eastern Kentucky. Sci. Total Environ. 408, 1628-1633 (2010).
15. Wang, X. et al. Frenolicins C-G, pyranonaphthoquinones from Streptomyces sp. RM-4-15. J. Nat. Prod. 76, 1441-1447 (2013).
16. Chresta, C. M. et al. AZD8055 is a potent, selective, and orally bioavailable ATP-competitive mammalian target of rapamycin kinase inhibitor with in vitro and in vivo antitumor activity. Cancer Res. 70, 288-298 (2010).
17. Laplante M. & Sabatini, D. M. mTOR signaling in growth control and disease. Cell 149, 274-293 (2012).
18. Choo, A. Y. & Blenis, J. Not all substrates are treated equally: implications for mTOR, rapamycin-resistance and cancer therapy. Cell Cycle 8, 567-572 (2009).
19. Feldman, M. E. et al. Active-site inhibitors of mTOR target rapamycin-resistant outputs of mTORC1 and mTORC2. PLoS Biol. 7, 371-383 (2009).
20. Toral-Barza, L. et al. Discovery of lactoquinomycin and related pyranonaphthoquinones as potent and allosteric inhibitors of AKT/PKB: mechanistic involvement of AKT catalytic activation loop cysteines. Mol. Cancer Ther. 6, 3028-3038 (2007).
21. Yap, T. A. et al. First-in-man clinical trial of the oral pan-AKT inhibitor MK-2206 in patients with advanced solid tumors. J. Clin. Oncol. 29, 4688-4695 (2011).
22. Zhang, Y. et al. A diastereoselective oxa-Pictet-Spengler-based strategy for (+)-frenolicin B and epi-(+)-frenolicin B synthesis. Org. Lett. 15, 5566-5569 (2013).,
23. Ellestad, G. A. Kunstmann. M. P., Whaley, H. A., & Patterson, E. L. The structure of frenolicin. J. Am. Chem. Soc. 90, 1325-1332 (1968).
24. Fitzgerald, J. T. et al. In vitro and in vivo activity of frenolicin B against Plasmodium falciparum and P berghei. J. Antibiot. 64, 799-801 (2011).
25. Sonenberg N, Hinnebusch A G. Regulation of translation initiation in eukaryotes: Mechanisms and biological targets. Cell. 2009; 136:731-45.
26. Koromilas A E, Lazaris-Karatzas A, Sonenberg N. mRNAs containing extensive secondary structure in their 5′ non-coding region translate efficiently in cells overexpressing initiation factor eIF-4E. EMBO J. 1992; 11:4153-8.
27. Graff J R, Konicek B W, Carter J H, Marcusson E G. Targeting the eukaryotic translation initiation factor 4E for cancer therapy. Cancer Res. 2008; 68:631-4.
28. De Benedetti A, Graff J R. eIF-4E expression and its role in malignancies and metastases. Oncogene. 2004; 23:3189-99.
29. Mamane Y, Petroulakis E, Martineau Y, Sato T A, Larsson 0, Rajasekhar V K, et al. Epigenetic activation of a subset of mRNAs by elF4E explains its effects on cell proliferation. PLoS One. 2007; 2:e242.
30. Silvera D, Formenti S C, Schneider R J. Translational control in cancer. Nat Rev Cancer. 2010; 10:254-66.
31. Hsieh A C, Liu Y, Edlind M P, Ingolia N T, Janes M R, Sher A, et al. The translational landscape of mTOR signaling steers cancer initiation and metastasis. Nature. 2012; 485:55-61.
32. Dilling M B, Germain G S, Dudkin L, Jayaraman A L, Zhang X, Harwood F C, et al. 4E-binding proteins, the suppressors of eukaryotic initiation factor 4E, are down-regulated in cells with acquired or intrinsic resistance to rapamycin. J Biol Chem. 2002; 277:13907-17.
33. Rosenwald I B, Chen J J, Wang S, Savas L, London I M, Pullman J. Upregulation of protein synthesis initiation factor elF-4E is an early event during colon carcinogenesis. Oncogene. 1999; 18:2507-17.
34. Berke! HJ, Turbat-Herrera E A, Shi R, de Benedetti A. Expression of the translation initiation factor elF4E in the polyp-cancer sequence in the colon. Cancer Epidemiol Biomarkers Prey. 2001; 10:663-6.
35. Martin M E, Perez M I, Redondo C, Alvarez M I, Salinas M, Fando J L. 4E binding protein 1 expression is inversely correlated to the progression of gastrointestinal cancers. Int J Biochem Cell Biol. 2000; 32:633-42.
36. Armengol G, Rojo F, Castellvi J, Iglesias C, Cuatrecasas M, Pons B, et al. 4E-binding protein 1: A key molecular “funnel factor” in human cancer with clinical implications. Cancer Res. 2007; 67:7551-5.
37. She Q B, Halilovic E, Ye Q, Zhen W, Shirasawa S, Sasazuki T, et al. 4E-BP1 is a key effector of the oncogenic activation of the AKT and ERK signaling pathways that integrates their function in tumors. Cancer Cell. 2010; 18:39-51.
38. Ye Q, Cai W, Zheng Y, Evers B M, She Q B. ERK and AKT signaling cooperate to translationally regulate survivin expression for metastatic progression of colorectal cancer. Oncogene. 2014; 33:1828-39.
39. Backman T W, Cao Y, Girke T. ChemMine tools: An online service for analyzing and clustering small molecules. Nucleic Acids Res. 2011; 39:W486-91.
40. Wang X, Shaaban K A, Elshahawi S I, Ponomareva L V, Sunkara M, Zhang Y, et al. Frenolicins C-G, pyranonaphthoquinones from Streptomyces sp. RM-4-15. J Nat Prod. 2013; 76:1441-7.
41. Zhang Y, Wang X, Sunkara M, Ye Q, Ponomereva L V, She Q B, et al. A diastereoselective oxa-Pictet-Spengler-based strategy for (+)-frenolicin B and epi-H-frenolicin B synthesis. Org Lett. 2013; 15:5566-9.
42. Jemal A, Bray F, Center M M, Ferlay J, Ward E, Forman D. Global cancer statistics. CA Cancer J Clin. 2011; 61:69-90.
43. Chu E. An update on the current and emerging targeted agents in metastatic colorectal cancer. Clin Colorectal Cancer. 2012; 11:1-13.
44. Shaaban K A, Wang X, Elshahawi S I, Ponomareva L V, Sunkara M, Copley G C, et al. Herbimycins D-F, ansamycin analogues from Streptomyces sp. RM-7-15. J Nat Prod. 2013; 76:1619-26.
45. Shaaban K A, Shepherd M D, Ahmed T A, Nybo S E, Leggas M, Rohr J. Pyramidamycins A-D and 3-hydroxyquinoline-2-carboxamide; cytotoxic benzamides from Streptomyces sp. DGC1. J Antibiot (Tokyo). 2012; 65:615-22.
46. Shaaban K A, Ahmed T A, Leggas M, Rohr J. Saquayamycins G-K, cytotoxic angucyclines from Streptomyces sp. Including two analogues bearing the aminosugar rednose. J Nat Prod. 2012; 75:1383-92.
47. Wang X, Elshahawi S I, Shaaban K A, Fang L, Ponomareva L V, Zhang Y, et al. Ruthmycin, a new tetracyclic polyketide from Streptomyces sp. RM-4-15. Org Lett. 2014; 16:456-9.
48. Shaaban K A, Singh S, Elshahawi S I, Wang X, Ponomareva L V, Sunkara M, et al. The native production of the sesquiterpene isopterocarpolone by Streptomyces sp. RM-14-6. Nat Prod Res. 2014; 28:337-9.
49. Shaaban K A, Singh S, Elshahawi S I, Wang X, Ponomareva L V, Sunkara M, et al. Venturicidin C, a new 20-membered macrolide produced by Streptomyces sp. TS-2-2. J Antibiot (Tokyo). 2014; 67:223-30.
50. Wang X, Shaaban K A, Elshahawi S I, Ponomareva L V, Sunkara M, Copley G C, et al. Mullinamides A and B, new cyclopeptides produced by the Ruth Mullins coal mine fire isolate Streptomyces sp. RM-27-46. J Antibiot (Tokyo). 2014; 67:571-5.
51. Savi D C, Shaaban K A, Vargas N, Ponomareva L V, Possiede Y M, Thorson J S, et al. Microbispora sp. LGMB259 endophytic actinomycete isolated from Vochysia divergens (Pantanal, Brazil) producing beta-carbolines and indoles with biological activity. Curr Microbiol. 2015; 70:345-54.
52. Manning B D, Tee A R, Logsdon M N, Blenis J, Cantley L C. Identification of the tuberous sclerosis complex-2 tumor suppressor gene product tuberin as a target of the phosphoinositide 3-kinase/akt pathway. Mol Cell. 2002; 10:151-62.
53. Ma L, Chen Z, Erdjument-Bromage H, Tempst P, Pandolfi P P. Phosphorylation and functional inactivation of TSC2 by Erk implications for tuberous sclerosis and cancer pathogenesis. Cell. 2005; 121:179-93.
54. Manning B D, Cantley L C. AKT/PKB signaling: Navigating downstream. Cell. 2007; 129:1261-74.
55. Zoncu R, Efeyan A, Sabatini D M. mTOR: From growth signal integration to cancer, diabetes and ageing. Nat Rev Mol Cell Biol. 2011; 12:21-35.
56. Choo A Y, Yoon S O, Kim S G, Roux P P, Blenis J. Rapamycin differentially inhibits S6Ks and 4E-BP1 to mediate cell-type-specific repression of mRNA translation. Proc Natl Acad Sci USA. 2008; 105:17414-9.
57. Choo A Y, Blenis J. Not all substrates are treated equally: Implications for mTOR, rapamycin-resistance and cancer therapy. Cell Cycle. 2009; 8:567-72.
58. Feldman M E, Apsel B, Uotila A, Loewith R, Knight Z A, Ruggero D, et al. Active-site inhibitors of mTOR target rapamycin-resistant outputs of mTORC1 and mTORC2. PLoS Biol. 2009; 7:e38.
59. Thoreen C C, Kang S A, Chang J W, Liu Q, Zhang J, Gao Y, et al. An ATP-competitive mammalian target of rapamycin inhibitor reveals rapamycin-resistant functions of mTORC1. J Biol Chem. 2009; 284:8023-32.
60. Kang S A, Pacold M E, Cervantes C L, Lim D, Lou H J, Ottina K, et al. mTORC1 phosphorylation sites encode their sensitivity to starvation and rapamycin. Science. 2013; 341:1236566.
61. Chresta C M, Davies B R, Hickson I, Harding T, Cosulich S, Critchlow S E, et al. AZD8055 is a potent, selective, and orally bioavailable ATP-competitive mammalian target of rapamycin kinase inhibitor with in vitro and in vivo antitumor activity. Cancer Res. 2010; 70:288-98.
62. Toral-Barza L, Zhang W G, Huang X, McDonald L A, Salaski E J, Barbieri L R, et al. Discovery of lactoquinomycin and related pyranonaphthoquinones as potent and allosteric inhibitors of AKT/PKB: Mechanistic involvement of AKT catalytic activation loop cysteines. Mol Cancer Ther. 2007; 6:3028-38.
63. Kovacina K S, Park G Y, Bae S S, Guzzetta A W, Schaefer E, Birnbaum M J, et al. Identification of a proline-rich Akt substrate as a 14-3-3 binding partner. J Biol Chem. 2003; 278:10189-94.
64. Hirai H, Sootome H, Nakatsuru Y, Miyama K, Taguchi S, Tsujioka K, et al. MK-2206, an allosteric Akt inhibitor, enhances antitumor efficacy by standard chemotherapeutic agents or molecular targeted drugs in vitro and in vivo. Mol Cancer Ther. 2010; 9:1956-67.
65. Yap T A, Yan L, Patnaik A, Fearen I, Olmos D, Papadopoulos K, et al. First-in-man clinical trial of the oral pan-AKT inhibitor MK-2206 in patients with advanced solid tumors. J Clin Oncol. 2011; 29:4688-95.
66. Eid C N, Shim J, Bikker J, Lin M. Direct oxa-Pictet-Spengler cyclization to the natural (3a,5)-trans-stereochemistry in the syntheses of (+)-7-deoxyfrenolicin B and (+)-7-deoxykalafungin. J Org Chem. 2009; 74:423-6.
67. Fernandes R A, Chavan V P. A DOtz benzannulation route to the enantioselective synthesis of (−)- and (+)-juglomycin A. Tetrahedron: Asymmetry. 2011; 22:1312-9.
68. Fernandes R A, Chavan V P, Mulay S V, Manchoju A. A chiron approach to the total synthesis of (−)-juglomycin A, (+)-kalafungin, (+)-frenolicin B, and (+)-deoxyfrenolicin. J Org Chem. 2012; 77:10455-60.
69. Xu Y C, Kohlman D T, Liang S X, Erikkson C. Stereoselective, oxidative C—C bond coupling of naphthopyran induced by DDQ: Stereocontrolled total synthesis of deoxyfrenolicin. Org Lett. 1999; 1:1599-602.
70. Mahlau M, Fernandes R A, Bruckner R. First synthesis of the pyrano-naphthoquinone lactone (−)-arizonin C1. Eur J Org Chem. 2011; 2011:4765-72.
71. Dong C, Yuan T, Wu Y, Wang Y, Fan T W, Miriyala S, et al. Loss of FBP1 by Snail-mediated repression provides metabolic advantages in basal-like breast cancer. Cancer Cell. 2013; 23:316-31.
72. Bolton J L, Trush M A, Penning T M, Dryhurst G, Monks T J. Role of quinones in toxicology. Chem Res Toxicol. 2000; 13:135-60.
73. Monks T J, Jones D C. The metabolism and toxicity of quinones, quinonimines, quinone methides, and quinone-thioethers. Curr Drug Metab. 2002; 3:425-38.
74. Wang X, Thomas B, Sachdeva R, Arterburn L, Frye L, Hatcher P G, et al. Mechanism of arylating quinone toxicity involving Michael adduct formation and induction of endoplasmic reticulum stress. Proc Natl Acad Sci USA. 2006; 103:3604-9.
75. Schieber M, Chandel N S. ROS function in redox signaling and oxidative stress. Curr Biol. 2014; 24:R453-62.
76. Ray P D, Huang B W, Tsuji Y. Reactive oxygen species (ROS) homeostasis and redox regulation in cellular signaling. Cell Signal. 2012; 24:981-90.
77. Bancirova M. Sodium azide as a specific quencher of singlet oxygen during chemiluminescent detection by luminol and Cypridina luciferin analogues. Luminescence. 2011; 26:685-8.
78. Dowling R J, Topisirovic I, Alain T, Bidinosti M, Fonseca B D, Petroulakis E, et al. mTORC1-mediated cell proliferation, but not cell growth, controlled by the 4E-BPs. Science. 2010; 328:1172-6.
79. Tsuji N, Kobayashi M, Wakisaka Y, Kawamura Y, Mayama M. New antibiotics, griseusins A and B. Isolation and characterization. J Antibiot (Tokyo). 1976; 29:7-9.
80. Tsuji N, Kobayashi M, Terui Y, Tori K. Structures of griseusins-A and griseusins-B, New Isochromanquinone Antibiotics. Tetrahedron. 1976; 32:2207-10.
81. Maruyama M, Nishida C, Takahashi Y, Naganawa H, Hamada M, Takeuchi T. 3′-O-alpha-D-forosaminyl-(+)-griseusin A from Streptomyces griseus. J Antibiot (Tokyo). 1994; 47:952-4.
82. Igarashi M, Chen W, Tsuchida T, Umekita M, Sawa T, Naganawa H, et al. 4′-Deacetyl-(−)-griseusins A and B, new naphthoquinone antibiotics from an actinomycete. J Antibiot (Tokyo). 1995; 48:1502-5.
83. Li Y Q, Li M G, Li W, Zhao J Y, Ding Z G, Cui X L, et al. Griseusin D, a new pyranonaphthoquinone derivative from a alkaphilic Nocardiopsis sp. J Antibiot (Tokyo). 2007; 60:757-61.
84. Ding Z G, Zhao J Y, Li M G, Huang R, Li Q M, Cui X L, et al. Griseusins F and G, spiro-naphthoquinones from a tin mine tailings-derived alkalophilic Nocardiopsis species. J Nat Prod. 2012; 75:1994-8.
85. He J, Roemer E, Lange C, Huang X, Maier A, Kelter G, et al. Structure, derivatization, and antitumor activity of new griseusins from Nocardiopsis sp. J Med Chem. 2007; 50:5168-75.
86. Kometani T, Takeuchi Y, Yoshii E. Pyranonaphthoquinone antibiotics. 4. Total synthesis of (+)-griseusin A, an enantiomer of the naturally occurring griseusin A. J Org Chem. 1983; 48:2311-4.
87. Brimble M A, Nairn M R, Park J. Synthesis of analogues of griseusin A. Org Lett. 1999; 1:1459-62.
88. Parker K A, Mindt T L, Koh Y H. TESOTf-induced rearrangement of quinols. Efficient construction of the fully functionalized carbon skeleton of the griseusins by a divergent-reconvergent approach. Org Lett. 2006; 8:1759-62.
89. Naysmith B J, Brimble M A. Synthesis of the griseusin B framework via a one-pot annulation-methylation-double deprotection-spirocyclization sequence. Org Lett. 2013; 15:2006-9.
90. Naysmith B J, Furkert D, Brimble M A. Synthesis of highly substituted pyranonaphthalene spiroketals related to the griseusins using a Hauser-Kraus annulation strategy. Tetrahedron. 2014; 70:1199-206.
91. Yu T W, Bibb M J, Revill W P, Hopwood D A. Cloning, sequencing, and analysis of the griseusin polyketide synthase gene cluster from Streptomyces griseus. J Bacteriol. 1994; 176:2627-34.
92. Gingras A C, Raught B, Gygi S P, Niedzwiecka A, Miron M, Burley S K, et al. Hierarchical phosphorylation of the translation inhibitor 4E-BP1. Genes Dev. 2001; 15:2852-64.
93. Herbert T P, Tee A R, Proud C G. The extracellular signal-regulated kinase pathway regulates the phosphorylation of 4E-BP1 at multiple sites. J Biol Chem. 2002; 277:11591-6.
94. Imai Y, Gehrke S, Wang H Q, Takahashi R, Hasegawa K, Oota E, et al. Phosphorylation of 4E-BP by LRRK2 affects the maintenance of dopaminergic neurons in Drosophila. EMBO J. 2008; 27:2432-43.
95. Michlewski G, Sanford J R, Caceres J F. The splicing factor SF2/ASF regulates translation initiation by enhancing phosphorylation of 4E-BP1. Mol Cell. 2008; 30:179-89.
96. Zhang Y, Zheng X F. mTOR-independent 4E-BP1 phosphorylation is associated with cancer resistance to mTOR kinase inhibitors. Cell Cycle. 2012; 11:594-603.
97. Patel M R, Sadiq A A, Jay-Dixon J, Jirakulaporn T, Jacobson B A, Farassati F, et al. Novel role of c-jun N-terminal kinase in regulating the initiation of cap-dependent translation. Int J Oncol. 2012; 40:577-82.
98. Schalm S S, Blenis J. Identification of a conserved motif required for mTOR signaling. Curr Biol. 2002; 12:632-9.
99. Schalm S S, Fingar D C, Sabatini D M, Blenis J. TOS motif-mediated raptor binding regulates 4E-BP1 multisite phosphorylation and function. Curr Biol. 2003; 13:797-806.

100. Sancak Y, Thoreen C C, Peterson T R, Lindquist R A, Kang S A, Spooner E, et al. PRAS40 is an insulin-regulated inhibitor of the mTORC1 protein kinase. Mol Cell. 2007; 25:903-15.

101. Grammel M, Hang H C. Chemical reporters for biological discovery. Nat Chem Biol. 2013; 9:475-84.

102. Thirumurugan P, Matosiuk D, Jozwiak K. Click chemistry for drug development and diverse chemical-biology applications. Chem Rev. 2013; 113:4905-79.

103. Morikawa K, Walker S M, Jessup J M, Fidler I J. In vivo selection of highly metastatic cells from surgical specimens of different primary human colon carcinomas implanted into nude mice. Cancer Res. 1988; 48:1943-8.

104. Moroz E, Carlin S, Dyomina K, Burke S, Thaler H T, Blasberg R, et al. Real-time imaging of HIF-1alpha stabilization and degradation. PLoS One. 2009; 4:e5077.

105. Zaytseva Y Y, Rychahou P G, Gulhati P, Elliott V A, Mustain W C, O'Connor K, et al. Inhibition of fatty acid synthase attenuates CD44-associated signaling and reduces metastasis in colorectal cancer. Cancer Res. 2012; 72:1504-17.

106. Gulhati P, Bowen K A, Liu J, Stevens P D, Rychahou P G, Chen M, et al. mTORC1 and mTORC2 regulate EMT, motility, and metastasis of colorectal cancer via RhoA and Racl signaling pathways. Cancer Res. 2011; 71:3246-56.

107. Cai W, Ye Q, She Q B. Loss of 4E-BP1 function induces EMT and promotes cancer cell migration and invasion via cap-dependent translational activation of snail. Oncotarget. 2014; 5:6015-27.

108. Teicher B A, editor. Anticancer drug development guide: Preclinical screening, clinical trials, and approval. 2 ed. Totowa, N.J.: Humana Press; 2004.

109. She Q B, Solit D B, Ye Q, O'Reilly K E, Lobo J, Rosen N. The BAD protein integrates survival signaling by EGFR/MAPK and PI3K/Akt kinase pathways in PTEN-deficient tumor cells. Cancer Cell. 2005; 8:287-97.

110. She Q B, Chandarlapaty S, Ye Q, Lobo J, Haskell K M, Leander K R, et al. Breast tumor cells with PI3K mutation or HER2 amplification are selectively addicted to Akt signaling. PLoS One. 2008; 3:e3065.

111. Rodrik-Outmezguine V S, Chandarlapaty S, Pagano N C, Poulikakos P I, Scaltriti M, Moskatel E, et al. mTOR kinase inhibition causes feedback-dependent biphasic regulation of AKT signaling. Cancer Discov. 2011; 1:248-59.

112. Bengalorkar G M, Bhuvana K, Sarala N, Kumar T. Fospropofol: Clinical pharmacology. J Anaesthesiol Clin Pharmacol. 2011; 27:79-83.

113. Boucher B A. Fosphenytoin: A novel phenytoin prodrug. Pharmacotherapy. 1996; 16:777-91.

114. Reinicke K E, Bey E A, Bentle M S, Pink J J, Ingalls S T, Hoppel C L, et al. Development of beta-lapachone prodrugs for therapy against human cancer cells with elevated NAD(P)H:quinone oxidoreductase 1 levels. Clin Cancer Res. 2005; 11:3055-64.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of selectively inhibiting 4E-BP1 phosphorylation comprising administering at least one pyranonaphthoquinone or pyranonaphthoquinone analog to a subject in need thereof.

2. The method of claim 1, wherein the at least one pyranonaphthoquinone or pyranonaphthoquinone analog includes a structure according to Formula I:

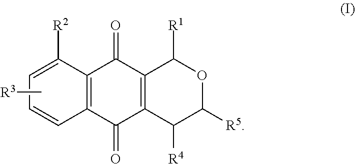

3. The method of claim 2, wherein the at least one pyranonaphthoquinone or pyranonaphthoquinone analog is a frenolicin or frenolicin analog.

4. The method of claim 3, wherein the frenolicin analog includes a structure according to Formula II:

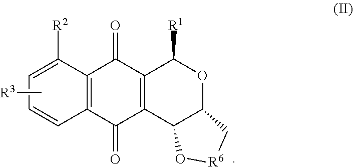

5. The method of claim 3, wherein the frenolicin analog includes a structure according to Formula III:

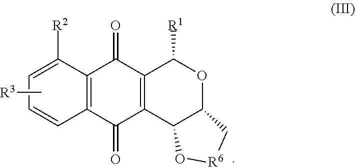

6. The method of claim 2, wherein the at least one pyranonaphthoquinone or pyranonaphthoquinone analog is a griseusin or griseusin analog.

7. The method of claim 6, wherein the griseusin or griseusin analog includes a structure according to formula IV:

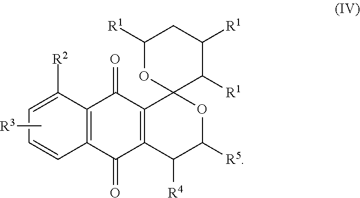

8. The method of claim 1, wherein administering the at least one pyranonaphthoquinone or pyranonaphthoquinone analog to the subject selectively inhibits 4E-BP1 phosphorylation.

9. The method of claim 1, wherein administering the at least one pyranonaphthoquinone or pyranonaphthoquinone analog to the subject modulates 4E-BP1-regulated cap-dependent translation.

10. A 4E-BP1 phosphorylation inhibitor comprising a pyranonaphthoquinone analog.

11. The inhibitor of claim 10, wherein the pyranonaphthoquinone analog comprises a griseusin analog.

12. The method of claim 11, wherein the griseusin analog includes a structure according to formula IV:

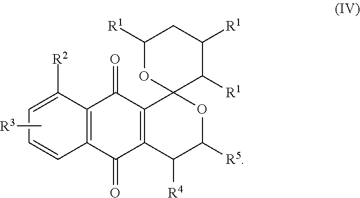

13. The inhibitor of claim 10, wherein the pyranonaphthoquinone analog comprises a frenolicin analog.

14. The method of claim 13, wherein the frenolicin analog includes a structure according to Formula II:

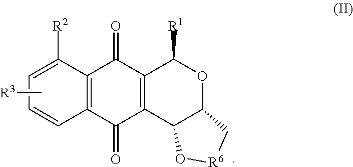

15. The method of claim 13, wherein the frenolicin analog includes a structure according to Formula III:

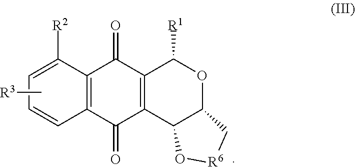

16. The inhibitor of claim 13, wherein the frenolicin analog is selected from the group consisting of an epi-frenolicin C1 analog, an epi-frenolicin ring A analog, an epi-frenolicin open D analog, and combinations thereof.

17. A method of treating cancer comprising administering at least one pyranonaphthoquinone or pyranonaphthoquinone analog to a subject in need thereof.

18. The method of claim 17, wherein the at least one pyranonaphthoquinone or pyranonaphthoquinone analog includes a structure according to Formula I:

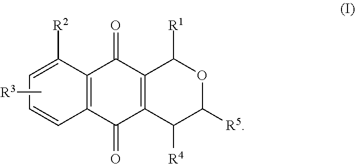

19. The method of claim 18, wherein the pyranonaphthoquinone analog comprises a griseusin analog.

20. The method of claim 18, wherein the pyranonaphthoquinone analog comprises a frenolicin analog.


SEQUENCE LISTING (6 sequences)

SEQ ID NO: 1 - protein, 199 aa, Homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 2 - protein, 335 aa, Homo sapiens
MAAGAAEAAVAAVEEVGSAGQFEELLRLKAKSLLVVHFWAPWAPQCAQMNEVMAELAKELPQVSFVKLEAEGVPEVSEKYEISSVPTFLFFKNSQKIDRLDGAHAPELTKKVQRHASSGSFLPSANEHLKEDLNLRLKKLTHAAPCMLFMKGTPQEPRCGFSKQMVEILHKHNIQFSSFDIFSDEEVRQGLKAYSSWPTYPQLYVSGELIGGLDIIKELEASEELDTICPKAPKLEERLKVLTNKASVMLFMKGNKQEAKCGFSKQILEILNSTGVEYETFDILEDEEVRQGLKAYSNWPTYPQLYVKGELVGGLDIVKELKENGELLPILRGEN

SEQ ID NO: 3 - DNA, 21 bp, Artificial
  antisense sequence for Prx1 shRNA
gctttcagtgatagggcagaa

SEQ ID NO: 4 - DNA, 21 bp, Artificial
  antisense sequence for Prx1 shRNA
gatgagactttgagactagtt

SEQ ID NO: 5 - DNA, 21 bp, Artificial
  antisense sequence for Grx3 shRNA
cctacctatcctcagctctat

SEQ ID NO: 6 - DNA, 21 bp, Artificial
  antisense sequence for Grx3 shRNA
gaacgaagttatggcagagtt